SEQUENCE LISTING (20 sequences)

SEQ ID NO: 1 - DNA, 100 bp, Artificial Sequence
  Description of Artificial Sequence Synthetic polynucleotide
  modified_base (63)..(68): a, c, t, g, unknown or other
  modified_base (100)..(100): a, c, t, g, unknown or other
tgtcgttgttgtcggccttctctggaagcagtggtatcaacgcagagtacggacgttcagagnnnnnnttttttttttttttttttttttttttttttvn

SEQ ID NO: 2 - DNA, 23 bp, Artificial Sequence
  Description of Artificial Sequence Synthetic oligonucleotide
aagcagtggtatcaacgcagagt

SEQ ID NO: 3 - DNA, 30 bp, Artificial Sequence
  Description of Artificial Sequence Synthetic oligonucleotide
  Description of Combined DNA/RNA Molecule Synthetic oligonucleotide
aagcagtggtatcaacgcagagtacatggg

SEQ ID NO: 4 - DNA, 15 bp, Artificial Sequence
  Description of Artificial Sequence Synthetic oligonucleotide
gtctcgtgggctcgg

SEQ ID NO: 5 - DNA, 47 bp, Artificial Sequence
  Description of Artificial Sequence Synthetic oligonucleotide
caagcagaagacggcatacgagattcgccttagtctcgtgggctcgg

SEQ ID NO: 6 - DNA, 66 bp, Artificial Sequence
  Description of Artificial Sequence Synthetic oligonucleotide
aatgatacggcgaccaccgagatctacacgcctgtccgcggaagcagtggtatcaacgcagagtac

SEQ ID NO: 7 - DNA, 68 bp, Artificial Sequence
  Description of Artificial Sequence Synthetic oligonucleotide
aatgatacggcgaccaccgagatctacacgcctgtccgcggaagcagtggtatcaacgcagagtacgg

SEQ ID NO: 8 - DNA, 28 bp, Artificial Sequence
  Description of Artificial Sequence Synthetic oligonucleotide
  modified_base (1)..(1): a, c, t, g, unknown or other
nbaaaaaaaaaaaaaaaaaaaaaaaaaa

SEQ ID NO: 9 - DNA, 30 bp, Artificial Sequence
  Description of Artificial Sequence Synthetic oligonucleotide
  Description of Combined DNA/RNA Molecule Synthetic oligonucleotide
cccatgtactctgcgttgataccactgctt

SEQ ID NO: 10 - DNA, 100 bp, Artificial Sequence
  Description of Artificial Sequence Synthetic polynucleotide
  modified_base (1)..(1): a, c, t, g, unknown or other
  modified_base (33)..(38): a, c, t, g, unknown or other
nvaaaaaaaaaaaaaaaaaaaaaaaaaaaaaannnnnnctctgaacgtccgtactctgcgttgataccactgcttccagagaaggccgacaacaacgaca

SEQ ID NO: 11 - DNA, 75 bp, Artificial Sequence
  Description of Artificial Sequence Synthetic oligonucleotide
  modified_base (1)..(1): a, c, t, g, unknown or other
  modified_base (33)..(38): a, c, t, g, unknown or other
nvaaaaaaaaaaaaaaaaaaaaaaaaaaaaaannnnnnctctgaacgtccgtactctgcgttgataccactgctt

SEQ ID NO: 12 - DNA, 75 bp, Artificial Sequence
  Description of Artificial Sequence Synthetic oligonucleotide
  modified_base (38)..(43): a, c, t, g, unknown or other
  modified_base (75)..(75): a, c, t, g, unknown or other
aagcagtggtatcaacgcagagtacggacgttcagagnnnnnnttttttttttttttttttttttttttttttvn

SEQ ID NO: 13 - DNA, 19 bp, Artificial Sequence
  Description of Artificial Sequence Synthetic oligonucleotide
ctgtctcttatacacatct

SEQ ID NO: 14 - DNA, 34 bp, Artificial Sequence
  Description of Artificial Sequence Synthetic oligonucleotide
gtctcgtgggctcggagatgtgtataagagacag

SEQ ID NO: 15 - DNA, 116 bp, Artificial Sequence
  Description of Artificial Sequence Synthetic polynucleotide
  modified_base (79)..(84): a, c, t, g, unknown or other
  modified_base (116)..(116): a, c, t, g, unknown or other
aatgatacggcgaccaccgagatctacacgcctgtccgcggaagcagtggtatcaacgcagagtacggacgttcagagnnnnnnttttttttttttttttttttttttttttttvn

SEQ ID NO: 16 - DNA, 34 bp, Artificial Sequence
  Description of Artificial Sequence Synthetic oligonucleotide
ctgtctcttatacacatctccgagcccacgagac

SEQ ID NO: 17 - DNA, 116 bp, Artificial Sequence
  Description of Artificial Sequence Synthetic polynucleotide
  modified_base (1)..(1): a, c, t, g, unknown or other
  modified_base (33)..(38): a, c, t, g, unknown or other
nvaaaaaaaaaaaaaaaaaaaaaaaaaaaaaannnnnnctctgaacgtccgtactctgcgttgataccactgcttccgcggacaggcgtgtagatctcggtggtcgccgtatcatt

SEQ ID NO: 18 - DNA, 66 bp, Artificial Sequence
  Description of Artificial Sequence Synthetic oligonucleotide
caagcagaagacggcatacgagattcgccttagtctcgtgggctcggagatgtgtataagagacag

SEQ ID NO: 19 - DNA, 66 bp, Artificial Sequence
  Description of Artificial Sequence Synthetic oligonucleotide
ctgtctcttatacacatctccgagcccacgagactaaggcgaatctcgtatgccgtcttctgcttg

SEQ ID NO: 20 - DNA, 37 bp, Artificial Sequence
  Description of Artificial Sequence Synthetic oligonucleotide
gcctgtccgcggaagcagtggtatcaacgcagagtac

(19) United States
(12) Patent Application Publication
(10) Pub. No.: US 2023/0220377 A1
(43) Pub. Date: Jul. 13, 2023

(54) SINGLE CELL ANALYSIS

(71) Applicant: BioSkryb Genomics, Inc., Durham, NC (US)

(72) Inventors: Jay A.A. WEST, Durham, NC (US); Charles GAWAD, Durham, NC (US)

(21) Appl. No.: 17/631,130

(22) PCT Filed: Jul. 30, 2020
(86) PCT No.: PCT/US2020/044338

§ 371 Date: Jan. 28, 2022

Related U.S. Application Data
(60) Provisional application No. 62/881,183, filed on Jul. 31, 2019.

(51) Int. Cl. C12N 15/10; C12Q 1/6806; C12Q 1/686; C12Q 1/6869

(52) U.S. Cl. CPC C12N 15/1093; C12N 15/1096; C12Q 1/6806; C12Q 1/686; C12Q 1/6869; C12Q 2600/156

(57) ABSTRACT

Provided herein are compositions and methods for accurate and scalable Primary Template-Directed Amplification (PTA) nucleic acid amplification and sequencing methods, and their applications for mutational analysis in research, diagnostics, and treatment. Further provided herein are multiomics methods for parallel analysis of DNA, RNA, and/or proteins from single cells. Provided herein are methods of multiomic single-cell analysis comprising: (a) isolating a single cell from a population of cells; (b) sequencing a cDNA library comprising polynucleotides amplified from mRNA transcripts from the single cell; and (c) sequencing a genome of the single cell.

### CROSS-REFERENCE

This application claims the benefit of U.S. provisional patent application number 62/881,183 filed on Jul. 31, 2019, which is incorporated herein by reference in its entirety.

### BACKGROUND

Research methods that utilize nucleic amplification, e.g., Next Generation Sequencing, provide large amounts of information on complex samples, genomes, and other nucleic acid sources. In some cases, these samples are obtained in small quantities from single cells. There is a need for highly accurate, scalable, and efficient nucleic acid amplification and sequencing methods for research, diagnostics, and treatment involving small samples, especially methods for simultaneous analysis of RNA, DNA, and proteins.

### BRIEF SUMMARY

Provided herein are methods of multiomic single-cell analysis comprising: (a) isolating a single cell from a population of cells; (b) sequencing a cDNA library comprising polynucleotides amplified from mRNA transcripts from the single cell; and (c) sequencing a genome of the single cell, wherein sequencing the genome comprises: (i) contacting the genome with at least one amplification primer, at least one nucleic acid polymerase, and a mixture of nucleotides, wherein the mixture of nucleotides comprises at least one terminator nucleotide which terminates nucleic acid replication by the polymerase, and (ii) amplifying at least some of the genome to generate a plurality of terminated amplification products, wherein the replication proceeds by strand displacement replication; (iii) ligating the molecules obtained in step (ii) to adaptors, thereby generating a genomic DNA library; and (iv) sequencing the genomic DNA library. Further provided herein are methods wherein the mRNA transcripts comprise polyadenylated mRNA transcripts. Further provided herein are methods wherein the mRNA transcripts do not comprise polyadenylated mRNA transcripts. Further provided herein are methods wherein sequencing a cDNA library comprises amplification of mRNA transcripts with template-switching primers. Further provided herein are methods wherein at least some of the polynucleotides of the cDNA library comprise a barcode. Further provided herein are methods wherein the barcode comprises a cell barcode or a sample barcode. Further provided herein are methods wherein the cDNA library and the genomic DNA library are pooled prior to sequencing. Further provided herein are methods wherein the single cell is a primary cell. Further provided herein are methods wherein the single cells originate from liver, skin, kidney, blood, or lung. Further provided herein are methods wherein the single cell is isolated by flow cytometry. Further provided herein are methods wherein the method further comprises removing at least one terminator nucleotide from the terminated amplification products. Further provided herein are methods wherein the plurality of terminated amplification products comprise an average of 1000-2000 bases in length. Further provided herein are methods wherein the plurality of terminated amplification products are 250-1500 bases in length. Further provided herein are methods wherein the plurality of terminated amplification products comprise at least 97% of the single cell's genome. Further provided herein are methods wherein at least some of the amplification products comprise a cell barcode or a sample barcode. Further provided herein are methods wherein sequencing a cDNA library comprises cystolic lysis of the single cell, and reverse transcription. Further provided herein are methods wherein the mRNA transcripts are amplified via template-switching reverse transcription. Further provided herein are methods wherein the cDNA library comprises at least 10,000 genes. Further provided herein are methods wherein sequencing a genome of the single cell further comprises nuclear lysis of the single cell. Further provided herein are methods wherein the method further comprises an additional amplification step using PCR. Further provided herein are methods wherein at least one mutation is identified in the genome of the cell, wherein the mutation differs from a corresponding position in a reference sequence. Further provided herein are methods wherein the at least one mutation occurs in less than 1% of the population of cells. Further provided herein are methods wherein the at least one mutation occurs in no more than 0.1% of the population of cells. Further provided herein are methods wherein the at least one mutation occurs in no more than 0.001% of the population of cells. Further provided herein are methods wherein the at least one mutation occurs in no more than 1% of the amplification product sequences. Further provided herein are methods wherein the at least one mutation occurs in no more than 0.1% of the amplification product sequences. Further provided herein are methods wherein the at least one mutation occurs in no more than 0.001% of the amplification product sequences.

Provided herein are methods of multiomic single-cell analysis comprising: (a) isolating a single cell from a population of cells; (b) identifying at least one protein on the surface of the single cell; and (c) sequencing a genome of the single cell, wherein sequencing the genome comprises: (i) contacting the genome with at least one amplification primer, at least one nucleic acid polymerase, and a mixture of nucleotides, wherein the mixture of nucleotides comprises at least one terminator nucleotide which terminates nucleic acid replication by the polymerase; (ii) amplifying at least some of the genome to generate a plurality of terminated amplification products, wherein the replication proceeds by strand displacement replication; (iii) ligating the molecules obtained in step (ii) to adaptors, thereby generating a genomic DNA library; and (iv) sequencing the genomic DNA library. Further provided herein are methods wherein identifying at least one protein on the cell surface comprises contacting the cell with a labeled antibody which binds to the at least one protein. Further provided herein are methods wherein the labeled antibody comprises at least one fluorescent label or mass-tag. Further provided herein are methods wherein the labeled antibody comprises at least one nucleic acid barcode.

Provided herein are methods of multiomic single-cell analysis comprising: (a) isolating a single cell from a population of cells; (b) sequencing a genome of the single cell, wherein sequencing the genome of the cell comprises: (i) digesting the genome with a methylation-sensitive restriction enzyme to generate genomic fragments; (ii) contacting at least some of the genomic fragments with at least one amplification primer, at least one nucleic acid polymerase, and a mixture of nucleotides, wherein the mixture of nucleotides comprises at least one terminator nucleotide which terminates nucleic acid replication by the polymerase; (iii) amplifying at least some of the genome to generate a plurality of terminated amplification products, wherein the replication proceeds by strand displacement replication; (iv) amplifying at least some of the genomic fragments with methylation-specific PCR; (v) ligating the molecules obtained in steps (iii and iv) to adaptors, thereby generating a genomic DNA library and a methylome DNA library; and (vi) sequencing the genomic DNA library and the methylome library.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a general workflow summary for isolation analysis of proteins, DNA, and RNA from single cells.

FIG. 1B illustrates a workflow for isolation analysis of proteins, DNA, and RNA from single cells using sample splitting to minimize cross-contamination.

FIG. 1C illustrates a workflow for isolation analysis of proteins, DNA, and RNA from single cells using single tube preamplification.

FIG. 1D illustrates a workflow for isolation analysis of proteins, DNA, and RNA from single cells using single tube preamplification with terminators to reduce amplicon size.

FIG. 1E illustrates a workflow for isolation analysis of proteins, DNA, and RNA from single cells using coamplification.

FIG. 1F illustrates an informatics workflow combining data from a protein/DNA/RNA single cell experiment described herein.

FIG. 1G illustrates a comparison of MDA and the PTA-Irreversible Terminator method as they relate to mutation propagation. The PTA method results in an increased number of direct copies of the original DNA template.

FIG. 2A illustrates method steps performed after amplification, which include removing the terminator, repairing ends, and performing A-tailing prior to adapter ligation. The library of pooled cells can then undergo hybridization-mediated enrichment for all exons or other specific regions of interest prior to sequencing. The cell of origin of each read is identified by the cell barcode (shown as green and blue sequences).

FIG. 2B (GC) shows comparison of GC content of sequenced bases for MDA and PTA experiments.

FIG. 2C shows map quality scores(e) (mapQ) mapping to human genome (p_mapped) after single cells underwent PTA or MDA.

FIG. 2D percent of reads mapping to human genome (p_mapped) after single cells underwent PTA or MDA.

FIG. 2E (PCR) shows the comparison of percent of reads that are PCR duplicates for 20 million subsampled reads after single cells underwent MDA and PTA.

FIG. 2F shows a workflow for RT amplification of single cells for use with PTA.

FIG. 2G shows creation of a library from cDNA obtained by RT.

FIG. 3A shows map quality scores(c) (mapQ2) mapping to human genome (p_mapped2) after single cells underwent PTA with reversible or irreversible terminators.

FIG. 3B shows percent of reads mapping to human genome (p_mapped2) after single cells underwent PTA with reversible or irreversible terminators.

FIG. 3C shows a series of box plots describing aligned reads for the mean percent reads overlapping with Alu elements using various methods. PTA had the highest number of reads aligned to the genome.

FIG. 3D shows a series of box plots describing PCR duplications for the mean percent reads overlapping with Alu elements using the various methods.

FIG. 3E shows a series of box plots describing GC content of reads for the mean percent reads overlapping with Alu elements using various methods.

FIG. 3F shows a series of box plots describing the mapping quality of mean percent reads overlapping with Alu elements using various methods. PTA had the highest mapping quality of methods tested.

FIG. 3G shows a comparison of SC mitochondrial genome coverage breadth with different WGA methods at a fixed 7.5× sequencing depth.

FIG. 4A shows mean coverage depth of 10 kilobase windows across chromosome 1 after selecting for a high-quality MDA cell (representative of ˜50% cells) compared to a random primer PTA-amplified cell after downsampling each cell to 40 million paired reads. The figure shows that MDA has less uniformity with many more windows that have more (box A) or less (box C) than twice the mean coverage depth. There is absence of coverage in both MDA and PTA at the centromere due to high GC content and low mapping quality of repetitive regions (box B).

FIG. 4B shows plots of sequencing coverage vs. genome position for MDA and PTA methods (top). The lower box plots show allele frequencies for MDA and PTA methods as compared to the bulk sample.

FIG. 5A shows a plot of the fraction of the genome covered vs. number of reads genome to evaluate the coverage at increasing sequencing depth for a variety of methods. The PTA method approaches the two bulk samples at every depth, which is an improvement over other methods tested.

FIG. 5B shows a plot of the coefficient of variation of the genome coverage vs. number of reads to evaluate coverage uniformity. The PTA method was found to have the highest uniformity of the methods tested.

FIG. 5C shows a Lorenz plot of the cumulative fraction of the total reads vs. the cumulative fraction of the genome. The PTA method was found to have the highest uniformity of the methods tested.

FIG. 5D shows a series of box plots of calculated Gini Indices for each of the methods tested in order to estimate the difference of each amplification reaction from perfect uniformity. The PTA method was found to be reproducibly more uniform than other methods tested.

FIG. 5E shows a plot of the fraction of bulk variants called vs. number of reads. Variant call rates for each of the methods were compared to the corresponding bulk sample at increasing sequencing depth. To estimate sensitivity, the percent of variants called in corresponding bulk samples that had been subsampled to 650 million reads found in each cell at each sequencing depth (FIG. 3A) were calculated. Improved coverage and uniformity of PTA resulted in the detection of 30% more variants over the Q-MDA method, which was the next most sensitive method.

FIG. 5F shows a series of box plots of the mean percent reads overlapping with Alu elements. The PTA method significantly diminished allelic skewing at these heterozygous sites. The PTA method more evenly amplifies two alleles in the same cell relative to other methods tested.

FIG. 5G shows a plot of specificity of variant calls vs. number of reads to evaluate the specificity of mutation calls. Variants found using various methods which were not found in the bulk samples were considered as false positives. The PTA method resulted in the lowest false positive calls (highest specificity) of methods tested.

FIG. 5H shows the fraction of false positive base changes for each type of base change across various methods. Without being bound by theory, such patterns may be polymerase dependent.

FIG. 5I shows a series of box plots of the mean percent reads overlapping with Alu elements for false positives variant calls. The PTA method resulted in the lowest allele frequencies for false positive variant calls.

FIG. 6 (part A) shows beads with oligonucleotides attached with a cleavable linker, unique cell barcode, and a random primer. Part B shows a single cell and bead encapsulated in the same droplet, followed by lysis of the cell and cleavage of the primer. The droplet may then be fused with another droplet comprising the PTA amplification mix. Part C shows droplets are broken after amplification, and amplicons from all cells are pooled. The protocol according to the disclosure is then utilized for removing the terminator, end repair, and A-tailing prior to adapter ligation. The library of pooled cells then undergoes hybridization-mediated enrichment for exons of interest prior to sequencing. The cell of origin of each read is then identified using the cell barcode.

FIG. 7A illustrates a workflow for multiomic (or polyomic) analysis of single cells using PTA. Step A: cells are contacted with antibodies comprising fluorescent labels and oligonucleotide barcode tags. Step B: Cells are sorted based on fluorescent markers. Step C: Tubes are coated with antibodies which bind to the nucleus; cells are lysed; cytosolic mRNA undergoes reverse transcription while the intact nucleus is bound to the tube wall.

FIG. 7B illustrates a workflow for multiomic analysis of single cells using PTA, continued from step C of FIG. 7A. Step D: after reverse transcription, the RT fraction is removed for sequencing analysis. Step E: the nucleus is lysed, and the PTA method is performed on the genomic DNA. Step F: PTA results in a short fragment cDNA pool with approximately 1000 fold amplification.

FIG. 8A illustrates primers used for reverse transcription and preamplification in a multiomic DNA/RNA single cell analysis workflow.

FIG. 8B illustrates a reverse transcription and preamplification workflow for a multiomic DNA/RNA single cell analysis workflow. Primers from FIG. 8A were used.

FIG. 9A illustrates a graph of growth rates for parental cell lines treated with 2 nM quizartinib for a period of three weeks to generate lines of AML cells that grow robustly in the presence of the FLT3 inhibitor. Single Resistant and Parental cells (FACS enriched) were then analyzed by RNA sequencing and low pass DNA sequencing analysis.

FIG. 9B illustrates RNA expression from both parental and resistant cultures demonstrated the ability to create cDNA pools (C) using the single-pot RNA seq chemistry and the genes expressed in these cells created distinct patterns that enable visualization of the cell populations by gene expression over the average of ˜10K genes detected per cell. In a separate workflow, single-cell genomes were amplified using the PTA method.

FIG. 9C illustrates normalized gene expression profiles for an RNAseq-only control experiment.

FIG. 9D illustrates a graph of the amount of amplified DNA by PTA vs. different protocols. Transcripts generated during the RT step (R) are not effectively amplified by the PTA reaction compared to DNA and the DNA in the single cells is effectively amplified using the combined protocol (SC1-SC8), compared to standard PTA amplified genomes from single cells (D, RD). NTC=no template control; R=RT step; D=PTA DNA step; RD=dual RT/PTA.

FIG. 10A illustrates mitochondrial chromosome amounts (%) for two different protocols (dual RNAseq/PTA, standard RNAseq) using a low pass sequencing protocol (˜5 million reads/cell).

and the estimate genome size was greater than 3 billion bases.

FIG. 10B illustrates the percent duplicates for two different protocols (dual RNAseq/PTA, standard RNAseq) using a low pass sequencing protocol (˜5 million reads/cell).

FIG. 10C illustrates the estimated genome size for two different protocols (dual RNAseq/PTA, standard RNAseq) using a low pass sequencing protocol (˜5 million reads/cell).

FIG. 10D illustrates feature assignments of 3 scRNAseq datasets from molm13 cells using the dual RNAseq/PTA protocol.

FIG. 10E illustrates a graph of normalized expression profiles for the Sum159 cell line obtained using a standard RNAseq protocol. P=parental cells. R=resistant cells.

FIG. 10F illustrates a graph of normalized expression profiles for the Sum159 cell line obtained using a dual RNAseq/PTA protocol. P=parental cells. R=resistant cells.

FIG. 11A shows the results of deep sequencing of 7 parental and 5 resistant molm13 cells performed to an approximate depth of 25× (K). The reads were aligned to Hg38 using bwa mem. Quality control and SNV-calling was performed using GATK4 best practices. SNVs were only considered if they were restricted to at least 2 resistant cells, no alternative alleles were called in any parental cell, and at least 6 parental cells were genotyped. All cells had at least 96% of the genome covered at 1× coverage and at least 76% covered at 10×. The inset shows that the known Flt3 indel in molm13 cells is detected in all cells (4 shown for clarity).

FIG. 11B illustrates a heat map of gene expression profiles, including overexpressed gene GAS6, which is a known mechanism of quizartinib resistance. Gas6 is the ligand for AXL, which is a clinically-relevant resistance mechanism in relapsed patients who fail quizartinib treatment.

FIG. 12A illustrates a graph of the proportion of covered exons in bulk versus single cell samples.

FIG. 12B illustrates a graph of the proportion of exons with no coverage in bulk versus single cell samples.

FIG. 12C illustrates a graph of percent selected bases in bulk versus single cell samples.

FIG. 12D illustrates a graph of the proportion of bases covered at 20× in bulk versus single cell samples.

FIG. 13A illustrates a graph of location of mapped read bases in the genome stratified by treatment and shaded by sample type.

FIG. 13B illustrates a graph of sample intensity vs. captured insert size.

FIG. 14A illustrates a graph of percent duplicates vs. percent selected bases for a 12-plex experiment.

FIG. 14B illustrates a graph of the number of target bases vs. coverage level.

### DETAILED DESCRIPTION OF THE INVENTION

There is a need to develop new scalable, accurate and efficient methods for nucleic acid amplification (including single-cell and multi-cell genome amplification) and sequencing which would overcome limitations in the current methods by increasing sequence representation, uniformity and accuracy in a reproducible manner. Provided herein are compositions and methods for providing accurate and scalable Primary Template-Directed Amplification (PTA) and sequencing. Further provided herein are methods of multiomic analysis, including analysis of proteins, DNA, and RNA from single cells, and corresponding post-transcriptional or post-translational modifications in combination with PTA. Such methods and compositions facilitate highly accurate amplification of target (or “template”) nucleic acids, which increases accuracy and sensitivity of downstream applications, such as Next-Generation Sequencing.

Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/− 10% thereof, or 10% below the lower listed limit and 10% above the higher listed limit for the values listed for a range.

The terms “subject” or “patient” or “individual”, as used herein, refer to animals, including mammals, such as, e.g., humans, veterinary animals (e.g., cats, dogs, cows, horses, sheep, pigs, etc.) and experimental animal models of diseases (e.g., mice, rats). In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A practical Approach, Volumes I and II (D. N. Glover ed. 1985); Oligonucleotide Synthesis (M J. Gait ed. 1984); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; B. Perbal, A practical Guide To Molecular Cloning (1984); F. M. Ausubel et al. (eds.), Current Protocols in Molecular Biology, John Wiley & Sons, Inc. (1994); among others.

The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acid acids, such as nucleic acid templates. Methods described herein additionally provide for the generation of isolated and at least partially purified nucleic acids and libraries of nucleic acids. In some instances, methods described herein provide for extracted nucleic acids (e.g., extracted from tissues, cells, or media). Nucleic acids include but are not limited to those comprising DNA, RNA, circular RNA, mtDNA (mitochondrial DNA), cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term “droplet” as used herein refers to a volume of liquid on a droplet actuator. Droplets in some instances, for example, be aqueous or non-aqueous or may be mixtures or emulsions including aqueous and non-aqueous components. For non-limiting examples of droplet fluids that may be subjected to droplet operations, see, e.g., Int. Pat. Appl. Pub. No. WO2007/120241. Any suitable system for forming and manipulating droplets can be used in the embodiments presented herein. For example, in some instances a droplet actuator is used. For non-limiting examples of droplet actuators which can be used, see, e.g., U.S. Pat. Nos. 6,911,132, 6,977,033, 6,773,566, 6,565,727, 7,163,612, 7,052,244, 7,328,979, 7,547,380, 7,641,779, U.S. Pat. Appl. Pub. Nos. US20060194331, US20030205632, US20060164490, US20070023292, US20060039823, US20080124252, US20090283407, US20090192044, US20050179746, US20090321262, US20100096266, US20110048951, Int. Pat. Appl. Pub. No. WO2007/120241. In some instances, beads are provided in a droplet, in a droplet operations gap, or on a droplet operations surface. In some instances, beads are provided in a reservoir that is external to a droplet operations gap or situated apart from a droplet operations surface, and the reservoir may be associated with a flow path that permits a droplet including the beads to be brought into a droplet operations gap or into contact with a droplet operations surface. Non-limiting examples of droplet actuator techniques for immobilizing magnetically responsive beads and/or non-magnetically responsive beads and/or conducting droplet operations protocols using beads are described in U.S. Pat. Appl. Pub. No. US20080053205, Int. Pat. Appl. Pub. No. WO2008/098236, WO2008/134153, WO2008/116221, WO2007/120241. Bead characteristics may be employed in the multiplexing embodiments of the methods described herein. Examples of beads having characteristics suitable for multiplexing, as well as methods of detecting and analyzing signals emitted from such beads, may be found in U.S. Pat. Appl. Pub. No. US20080305481, US20080151240, US20070207513, US20070064990, US20060159962, US20050277197, US20050118574.

Primers and/or template switching oligonucleotides can also be affixed to solid substrate to facilitate reverse transcription and template switching of the mRNA polynucleotides. In this arrangement a portion of the RT or template switching reaction occurs in the bulk solution of the device, where the second step of the reaction occurs in proximity to the surface. In other arrangements the primer of template switch oligonucleotide is allowed to be released from the solid substrate to allow the entire reaction to occur above the surface in the solution. In a polyomic approach the primers for the multistage reaction in some instances is affixed to the solid substrate or combined with beads to accomplish combinations of multistage primers.

Certain microfluidic devices also support polyomic approaches. Devices fabricated in PDMS, as an example, often have contiguous chambers for each reaction step. Such multichambered devices are often segregated using a microvalve structure which can be controlled though the pressure with air, or a fluid such as water or inert hydrocarbon (i.e. fluorinert). In a multiomic approach each stage of the reaction can be sequestered and allowed to be conducted discretely. At the completion of a particular stage a valve between an adjacent chamber can be released on the substrates for the subsequent reaction can be added in a serial fashion. The result is the ability to emulate an sequential set of reactions, such as a multiomic (Protein/RNA/DNA/epigenomic) set of reactions using an individual cell as a input template material. Various microfluidics platforms may be used for analysis of single cells. Cells in some instances are manipulated through hydrodynamics (droplet microfluidics, inertial microfluidics, vortexing, microvalves, microstructures (e.g., microwells, microtraps)), electrical methods (dielectrophoresis (DEP), electroosmosis), optical methods (optical tweezers, optically induced dielectrophoresis (ODEP), opto-thermocapillary), acoustic methods, or magnetic methods. In some instances, the microfluidics platform comprises microwells. In some instances, the microfluidics platform comprises a PDMS (Polydimethylsiloxane)-based device. Non-limited examples of single cell analysis platforms compatible with the methods described herein are: ddSEQ Single-Cell Isolator, (Bio-Rad, Hercules, Calif., USA, and Illumina, San Diego, Calif., USA)); Chromium (10× Genomics, Pleasanton, Calif., USA)); Rhapsody Single-Cell Analysis System (BD, Franklin Lakes, N.J., USA); Tapestri Platform (MissionBio, San Francisco, Calif., USA)), Nadia Innovate (Dolomite Bio, Royston, UK); Cl and Polaris (Fluidigm, South San Francisco, Calif., USA); ICELL8 Single-Cell System (Takara); MSND (Wafergen); Puncher platform (Vycap); CellRaft AIR System (CellMicrosystems); DEPArray NxT and DEPArray System (Menarini Silicon Biosystems); AVISO CellCelector (ALS); and InDrop System (1CellBio), and TrapTx (Celldom).

As used herein, the term “unique molecular identifier (UMI)” refers to a unique nucleic acid sequence that is attached to each of a plurality of nucleic acid molecules. When incorporated into a nucleic acid molecule, an UMI in some instances is used to correct for subsequent amplification bias by directly counting UMIs that are sequenced after amplification. The design, incorporation and application of UMIs is described, for example, in Int. Pat. Appl. Pub. No. WO 2012/142213, Islam et al. Nat. Methods (2014) 11:163-166, Kivioja, T. et al. Nat. Methods (2012) 9: 72-74, Brenner et al. (2000) PNAS 97(4), 1665, and Hollas and Schuler, (2003) Conference: 3rd International Workshop on Algorithms in Bioinformatics, Volume: 2812.

As used herein, the term “barcode” refers to a nucleic acid tag that can be used to identify a sample or source of the nucleic acid material. Thus, where nucleic acid samples are derived from multiple sources, the nucleic acids in each nucleic acid sample are in some instances tagged with different nucleic acid tags such that the source of the sample can be identified. Barcodes, also commonly referred to indexes, tags, and the like, are well known to those of skill in the art. Any suitable barcode or set of barcodes can be used. See, e.g., non-limiting examples provided in U.S. Pat. No. 8,053,192 and Int. Pat. Appl. Pub. No. WO2005/068656. Barcoding of single cells can be performed as described, for example, in U.S. Pat. Appl. Pub. No. 2013/0274117.

The terms “solid surface,” “solid support” and other grammatical equivalents herein refer to any material that is appropriate for or can be modified to be appropriate for the attachment of the primers, barcodes and sequences described herein. Exemplary substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon, nitrocellulose, ceramics, resins, silica, silica-based materials (e.g., silicon or modified silicon), carbon, metals, inorganic glasses, plastics, optical fiber bundles, and a variety of other polymers. In some embodiments, the solid support comprises a patterned surface suitable for immobilization of primers, barcodes and sequences in an ordered pattern.

As used herein, the term “biological sample” includes, but is not limited to, tissues, cells, biological fluids and isolates thereof. Cells or other samples used in the methods described herein are in some instances isolated from human patients, animals, plants, soil or other samples comprising microbes such as bacteria, fungi, protozoa, etc. In some instances, the biological sample is of human origin. In some instances, the biological is of non-human origin. The cells in some instances undergo PTA methods described herein and sequencing. Variants detected throughout the genome or at specific locations can be compared with all other cells isolated from that subject to trace the history of a cell lineage for research or diagnostic purposes. In some instances, variants are confirmed through additional methods of analysis such as direct PCR sequencing.

Single Cell Analysis

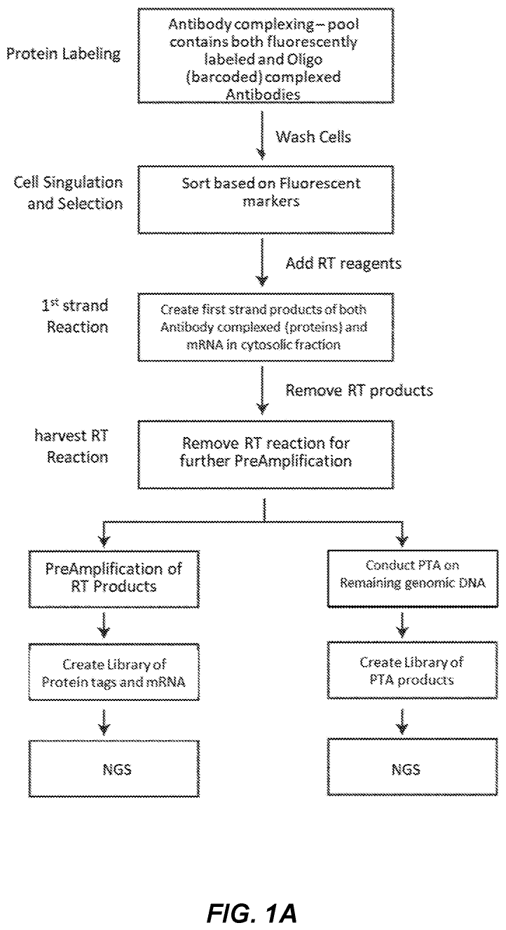

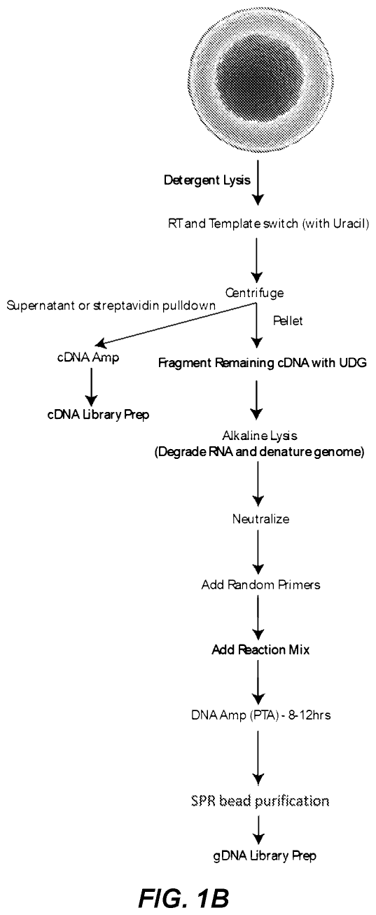

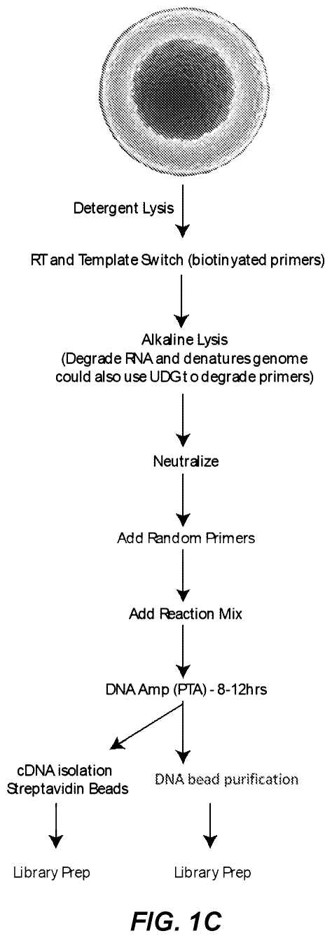

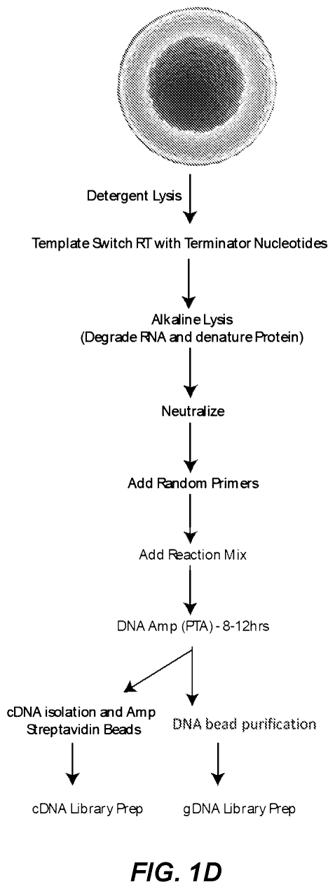

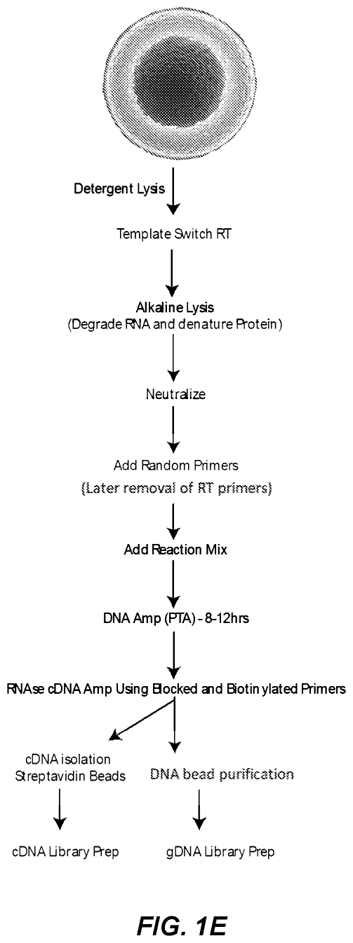

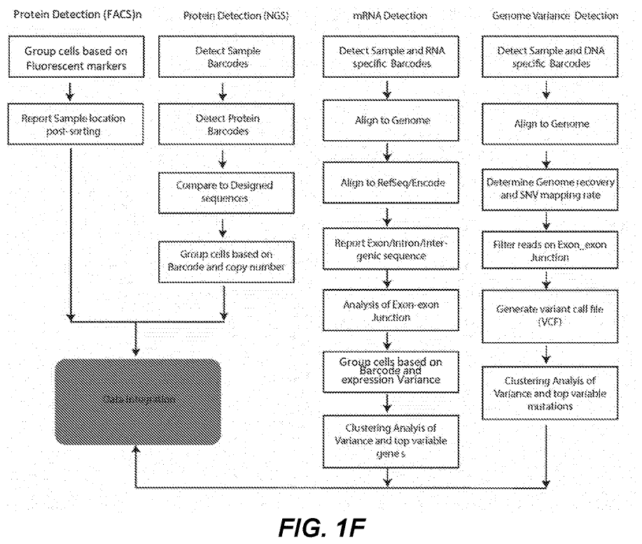

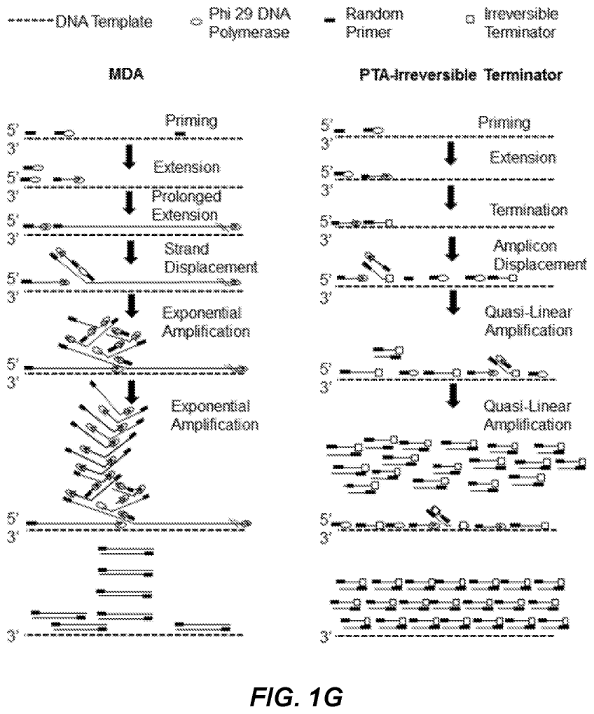

Described herein are methods and compositions for analysis of single cells. Analysis of cells in bulk provides general information about the cell population, but often is unable to detect low-frequency mutants over the background. Such mutants may comprise important properties such as drug resistance or mutations associated with cancer. In some instances, DNA, RNA, and/or proteins from the same single cell are analyzed in parallel. The analysis may include identification of epigenetic post-translational (e.g., glycosylation, phosphorylation, acetylation, ubiquination, histone modification) and/or post-transcriptional (e.g., methylation, hydroxymethylation) modifications. Such methods may comprise “Primary Template-Directed Amplification” (PTA) to obtain libraries of nucleic acids for sequencing. In some instances PTA is combined with additional steps or methods such as RT-PCR or proteome/protein quantification techniques (e.g., mass spectrometry, antibody staining, etc.). In some instances, various components of a cell are physically or spatially separated from each other during individual analysis steps. For example, a workflow in some instances comprises the general steps in FIG. 1A. Proteins are first labeled with antibodies. In some instances, at least some of the antibodies comprise a tag or marker (e.g., nucleic acid/oligo tag, mass tag, or fluorescent, tag). In some instances, a portion of the antibodies comprise an oligo tag. In some instances, a portion of the antibodies comprise a fluorescent marker. In some instances antibodies are labeled by two or more tags or markers. In some instances, a portion of the antibodies are sorted based on fluorescent markers. After RT-PCR, first strand mRNA products are generated and then removed for analysis. Libraries are then generated from RT-PCR products and barcodes present on protein-specific antibodies, which are subsequently sequenced. In parallel, genomic DNA from the same cell is subjected to PTA, a library generated, and sequenced. Sequencing results from the genome, proteome, and transcriptome are in some instances pooled using bioinformatics methods. Methods described herein in some instances comprise any combination of labeling, cell sorting, affinity separation/purification, lysing of specific cell components (e.g., outer membrane, nucleus, etc.), RNA amplification, DNA amplification (e.g., PTA), or other step associated with protein, RNA, or DNA isolation or analysis. In some instances, methods described herein comprise one or more enrichment steps, such as exome enrichment.

Described herein is a first method of single cell analysis comprising analysis of RNA and DNA from a single cell (FIG. 1B). In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. Alternatively or in combination, centrifugation is used to separate RNA in the supernatant from cDNA in the cell pellet. Remaining cDNA is in some instances fragmented and removed with UDG (uracil DNA glycosylase), and alkaline lysis is used to degrade RNA and denature the genome. After neutralization, addition of primers and PTA, amplification products are in some instances purified on SPRI (solid phase reversible immobilization) beads, and ligated to adapters to generate a gDNA library.

Described herein is a second method of single cell analysis comprising analysis of RNA and DNA from a single cell (FIG. 1C). In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a gDNA library. RT products are in some instances isolated by pulldown, such as a pulldown with streptavidin beads.

Described herein is a third method of single cell analysis comprising analysis of RNA and DNA from a single cell (FIG. 1D). In some instances, the method comprises isolation of single cells, lysis of single cells, and reverse transcription (RT). In some instances, reverse transcription is carried out with template switching oligonucleotides (TSOs) in the presence of terminator nucleotides. In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a DNA library. RT products are in some instances isolated by pulldown, such as a pulldown with streptavidin beads.

Described herein is a fourth method of single cell analysis comprising analysis of RNA and DNA from a single cell (FIG. 1E). In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances subjected to RNase and cDNA amplification using blocked and labeled primers. gDNA is purified on SPRI (solid phase reversible immobilization) beads, and ligated to adapters to generate a gDNA library. RT products are in some instances are isolated by pulldown, such as a pulldown with streptavidin beads.

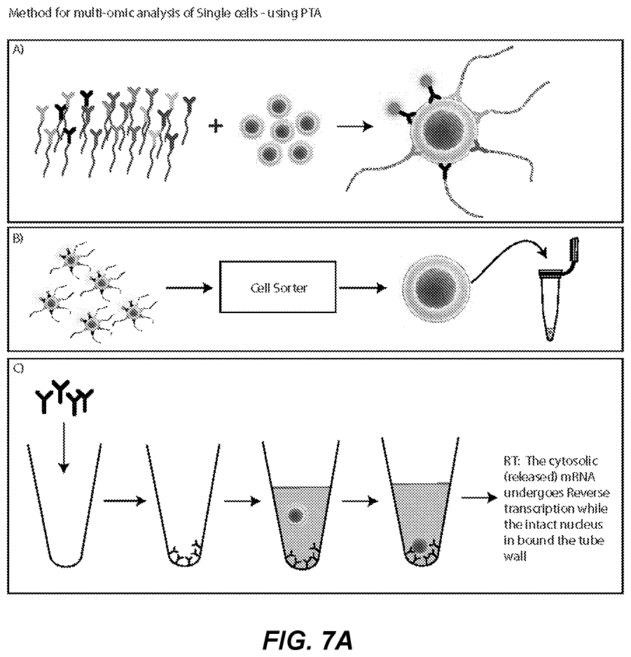

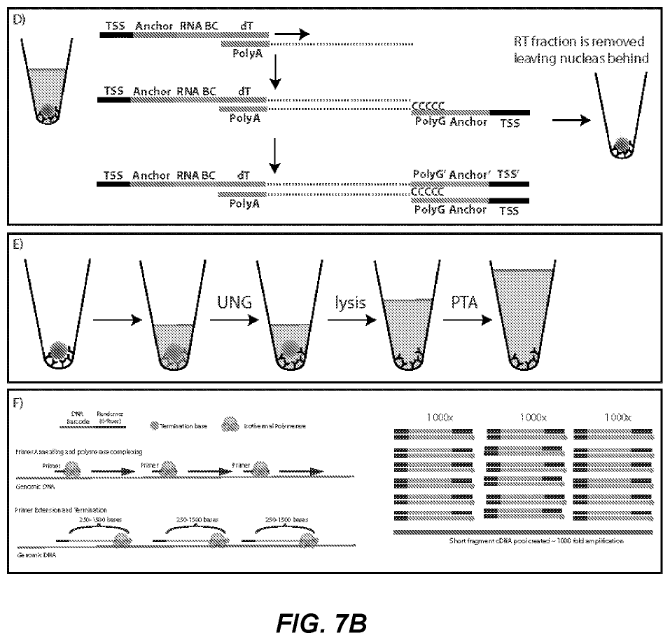

Described herein is a fifth method of single cell analysis comprising analysis of RNA and DNA from a single cell (FIGS. 7A and 7B). A population of cells is contacted with an antibody library, wherein antibodies are labeled. In some instances, antibodies are labeled with either fluorescent labels, nucleic acid barcodes, or both. Labeled antibodies bind to at least one cell in the population, and such cells are sorted, placing one cell per container (e.g., a tube, vial, microwell, etc.). In some instances, the container comprises a solvent. In some instances, a region of a surface of a container is coated with a capture moiety. In some instances, the capture moiety is a small molecule, an antibody, a protein, or other agent capable of binding to one or more cells, organelles, or other cell component. In some instances, at least one cell, or a single cell, or component thereof, binds to a region of the container surface. In some instances, a nucleus binds to the region of the container. In some instances, the outer membrane of the cell is lysed, releasing mRNA into a solution in the container. In some instances, the nucleus of the cell containing genomic DNA is bound to a region of the container surface. Next, RT is often performed using the mRNA in solution as a template to generate cDNA. In some instances, template switching primers comprise from 5′ to 3′ a TSS region (transcription start site), an anchor region, a RNA BC region, and a poly dT tail. In some instances, the poly dT tail binds to poly A tail of one or more mRNAs. In some instances, template switching primers comprise from 3′ to 5′ a TSS region, an anchor region, and a poly G region. In some instances, the poly G region comprises riboG. In some instances the poly G region binds to a poly C region on an mRNA transcript. In some instances, riboG was added to the mRNA transcripts by a terminal transferase. After removal of RT PCR products for subsequent sequencing, any remaining RNA in the cell is removed by UNG. The nucleus is then lysed, and the released genomic DNA is subjected to the PTA method using random primers with an isothermal polymerase. In some instances, primers are 6-9 bases in length. In some instances, PTA generates genomic amplicons of 100-5000, 200-5000, 500-2000, 500-2500, 1000-3000, or 300-3000 bases in length. In some instances, PTA generates genomic amplicons with an average length of 100-5000, 200-5000, 500-2000, 500-2500, 1000-3000, or 300-3000 bases. In some instances, PTA generates genomic amplicons of 250-1500 bases in length. In some instances, the methods described herein generate a short fragment cDNA pool with about 500, about 750, about 1000, about 5000, or about 10,000 fold amplification. In some instances, the methods described herein generate a short fragment cDNA pool with 500-5000, 750-1500, or 250-10,000 fold amplification. PTA products are optionally subjected to additional amplification and sequenced.

Sample Preparation and Isolation of Single Cells

Methods described herein may require isolation of single cells for analysis. Any method of single cell isolation may be used with PTA, such as mouth pipetting, micro pipetting, flow cytometry/FACS, microfluidics, methods of sorting nuclei (tetraploid or other), or manual dilution. Such methods are aided by additional reagents and steps, for example, antibody-based enrichment (e.g., circulating tumor cells), other small-molecule or protein-based enrichment methods, or fluorescent labeling. In some instances, a method of multiomic analysis described herein comprises mechanical or enzymatic dissociate of cells from larger tissues.

Preparation and Analysis of Cell Components

Methods of multiomic analysis comprising PTA described herein may comprise one or more methods of processing cell components such as DNA, RNA, and/or proteins. In some instances, the nucleus (comprising genomic DNA) is physically separated from the cytosol (comprising mRNA), followed by a membrane-selective lysis buffer to dissolve the membrane but keep the nucleus intact. The cytosol is then separated from the nucleus using methods including micro pipetting, centrifugation, or anti-body conjugated magnetic microbeads. In another instance, an oligo-dT primer coated magnetic bead binds polyadenylated mRNA for separation from DNA. In another instance, DNA and RNA are preamplified simultaneously, and then separated for analysis. In another instance, a single cell is split into two equal pieces, with mRNA from one half processed, and genomic DNA from the other half processed.

Multiomics

Methods described herein (e.g., PTA) may be used as a replacement for any number of other known methods in the art which are used for single cell sequencing (multiomics or the like). PTA may substitute genomic DNA sequencing methods such as MDA, PicoPlex, DOP-PCR, MALBAC, or target-specific amplifications. In some instances, PTA replaces the standard genomic DNA sequencing method in a multiomics method including DR-seq (Dey et al., 2015), G&T seq (MacAulay et al., 2015), scMT-seq (Hu et al., 2016), sc-GEM (Cheow et al., 2016), scTrio-seq (Hou et al., 2016), simultaneous multiplexed measurement of RNA and proteins (Darmanis et al., 2016), scCOOL-seq (Guo et al., 2017), CITE-seq (Stoeckius et al., 2017), REAP-seq (Peterson et al., 2017), scNMT-seq (Clark et al., 2018), or SIDR-seq (Han et al., 2018). In some instances, a method described herein comprises PTA and a method of polyadenylated mRNA transcripts. In some instances, a method described herein comprises PTA and a method of non-polyadenylated mRNA transcripts. In some instances, a method described herein comprises PTA and a method of total (polyadenylated and non-polyadenylated) mRNA transcripts.

In some instances, PTA is combined with a standard RNA sequencing method to obtain genome and transcriptome data. In some instances, a multiomics method described herein comprises PTA and one of the following: Drop-seq (Macosko, et al. 2015), mRNA-seq (Tang et al., 2009), InDrop (Klein et al., 2015), MARS-seq (Jaitin et al., 2014), Smart-seq2 (Hashimshony, et al., 2012; Fish et al., 2016), CEL-seq (Jaitin et al., 2014), STRT-seq (Islam, et al., 2011), Quartz-seq (Sasagawa et al., 2013), CEL-seq2 (Hashimshony, et al. 2016), cytoSeq (Fan et al., 2015), SuPeR-seq (Fan et al., 2011), RamDA-seq (Hayashi, et al. 2018), MATQ-seq (Sheng et al., 2017), or SMARTer (Verboom et al., 2019).

Various reaction conditions and mixes may be used for generating cDNA libraries for transcriptome analysis. In some instances, an RT reaction mix is used to generate a cDNA library. In some instances, the RT reaction mixture comprises a crowding reagent, at least one primer, a template switching oligonucleotide (TSO), a reverse transcriptase, and a dNTP mix. In some instances, an RT reaction mix comprises an RNAse inhibitor. In some instances an RT reaction mix comprises one or more surfactants. In some instances an RT reaction mix comprises Tween-20 and/or Triton-X. In some instances an RT reaction mix comprises Betaine. In some instances an RT reaction mix comprises one or more salts. In some instances an RT reaction mix comprises a magnesium salt (e.g., magnesium chloride) and/or tetramethylammonium chloride. In some instances an RT reaction mix comprises gelatin. In some instances an RT reaction mix comprises PEG (PEG1000, PEG2000, PEG4000, PEG6000, PEG8000, or PEG of other length).

Multiomic methods described herein may provide both genomic and RNA transcript information from a single cell (e.g., a combined or dual protocol). In some instances, genomic information from the single cell is obtained from the PTA method, and RNA transcript information is obtained from reverse transcription to generate a cDNA library. In some instances, a whole transcript method is used to obtain the cDNA library. In some instances, 3′ or 5′ end counting is used to obtain the cDNA library. In some instances, cDNA libraries are not obtained using UMIs. In some instances, a multiomic method provides RNA transcript information from the single cell for at least 500, 1000, 2000, 5000, 8000, 10,000, 12,000, or at least 15,000 genes. In some instances, a multiomic method provides RNA transcript information from the single cell for about 500, 1000, 2000, 5000, 8000, 10,000, 12,000, or about 15,000 genes. In some instances, a multiomic method provides RNA transcript information from the single cell for 100-12,000 1000-10,000, 2000-15,000, 5000-15,000, 10,000-20,000, 8000-15,000, or 10,000-15,000 genes. In some instances, a multiomic method provides genomic sequence information for at least 80%, 90%, 92%, 95%, 97%, 98%, or at least 99% of the genome of the single cell. In some instances, a multiomic method provides genomic sequence information for about 80%, 90%, 92%, 95%, 97%, 98%, or about 99% of the genome of the single cell.

Multiomic methods may comprise analysis of single cells from a population of cells. In some instances, at least 5, 10, 20, 50, 100, 200, 500, 1000, 2000, 5000, or at least 8000 cells are analyzed. In some instances, about 5, 10, 20, 50, 100, 200, 500, 1000, 2000, 5000, or about 8000 cells are analyzed. In some instances, 5-100, 10-100, 50-500, 100-500, 100-1000, 50-5000, 100-5000, 500-1000, 500-10000, 1000-10000, or 5000-20,000 cells are analyzed.

Multiomic methods may generate yields of genomic DNA from the PTA reaction based on the type of single cell. In some instances, the amount of DNA generated from a single cell is about 0.1, 1, 1.5, 2, 3, 5, or about 10 micrograms. In some instances, the amount of DNA generated from a single cell is about 0.1, 1, 1.5, 2, 3, 5, or about 10 femtograms. In some instances, the amount of DNA generated from a single cell is at least 0.1, 1, 1.5, 2, 3, 5, or at least 10 micrograms. In some instances, the amount of DNA generated from a single cell is at least 0.1, 1, 1.5, 2, 3, 5, or at least 10 femtograms. In some instances, the amount of DNA generated from a single cell is about 0.1-10, 1-10, 1.5-10, 2-20, 2-50, 1-3, or 0.5-3.5 micrograms. In some instances, the amount of DNA generated from a single cell is about 0.1-10, 1-10, 1.5-10, 2-20, 2-4, 1-3, or 0.5-4 femtograms.

Methylome Analysis

Described herein are methods comprising PTA, wherein sites of methylated DNA in single cells are determined using the PTA method. In some instances, these methods further comprise parallel analysis of the transcriptome and/or proteome of the same cell. Methods of detecting methylated genomic bases include selective restriction with methylation-sensitive endonucleases, followed by processing with the PTA method. Sites cut by such enzymes are determined from sequencing, and methylated bases are identified. In another instance, bisulfite treatment of genomic DNA libraries converts unmethylated cytosines to uracil. Libraries are then in some instances amplified with methylation-specific primers which selectively anneal to methylated sequences. Alternatively, non-methylation-specific PCR is conducted, followed by one or more methods to discriminate between bisulfite-reacted bases, including direct pyrosequencing, MS-SnuPE, HRM, COBRA, MS-SSCA, or base-specific cleavage/MALDI-TOF. In some instances, genomic DNA samples are split for parallel analysis of the genome (or an enriched portion thereof) and methylome analysis. In some instances, analysis of the genome and methylome comprises enrichment of genomic fragments (e.g., exome, or other targets) or whole genome sequencing.

Bioinformatics

The data obtained from single-cell analysis methods utilizing PTA described herein may be compiled into a database. Described herein are methods and systems of bioinformatic data integration. Data from the proteome, genome, transcriptome, methylome or other data is in some instances combined/integrated into a database and analyzed. Bioinformatic data integration methods and systems in some instances comprise one or more of protein detection (FACS and/or NGS), mRNA detection, and/or genome variance detection. In some instances, this data is correlated with a disease state or condition. In some instances, data from a plurality of single cells is compiled to describe properties of a larger cell population, such as cells from a specific sample, region, organism, or tissue. In some instances, protein data is acquired from fluorescently labeled antibodies which selectively bind to proteins on a cell. In some instances, a method of protein detection comprises grouping cells based on fluorescent markers and reporting sample location post-sorting. In some instances, a method of protein detection comprises detecting sample barcodes, detecting protein barcodes, comparing to designed sequences, and grouping cells based on barcode and copy number. In some instances, protein data is acquired from barcoded antibodies which selectively bind to proteins on a cell. In some instances, transcriptome data is acquired from sample and RNA specific barcodes. In some instances, a method of mRNA detection comprises detecting sample and RNA specific barcodes, aligning to genome, aligning to RefSeq/Encode, reporting Exon/Intro/Intergenic sequences, analyzing exon-exon junctions, grouping cells based on barcode and expression variance and clustering analysis of variance and top variable genes. In some instances, genomic data is acquired from sample and DNA specific barcodes. In some instances, a method of genome variance detection comprises detecting sample and DNA specific barcodes, aligning to the genome, determine genome recovery and SNV mapping rate, filtering reads on exon-exon junctions, generating variant call file (VCF), and clustering analysis of variance and top variable mutations.

Mutations

In some instances, the methods (e.g., multiomic PTA) described herein result in higher detection sensitivity and/or lower rates of false positives for the detection of mutations. In some instances a mutation is a difference between an analyzed sequence (e.g., using the methods described herein) and a reference sequence. Reference sequences are in some instances obtained from other organisms, other individuals of the same or similar species, populations of organisms, or other areas of the same genome. In some instances, mutations are identified on a plasmid or chromosome. In some instances, a mutation is an SNV (single nucleotide variation), SNP (single nucleotide polymorphism), or CNV (copy number variation, or CNA/copy number aberration). In some instances, a mutation is base substitution, insertion, or deletion. In some instances, a mutation is a transition, transversion, nonsense mutation, silent mutation, synonymous or non-synonymous mutation, non-pathogenic mutation, missense mutation, or frameshift mutation (deletion or insertion). In some instances, PTA results in higher detection sensitivity and/or lower rates of false positives for the detection of mutations when compared to methods such as in-silico prediction, ChIP-seq, GUIDE-seq, circle-seq, HTGTS (High-Throughput Genome-Wide Translocation Sequencing), DLV (integration-deficient lentivirus), Digenome-seq, FISH (fluorescence in situ hybridization), or DISCOVER-seq.

Primary Template-Directed Amplification

Described herein are nucleic acid amplification methods, such as “Primary Template-Directed Amplification (PTA).” In some instances, PTA is combined with other analysis workflows for multiomic analysis. For example, one embodiment of the PTA method described herein are schematically represented in FIG. 1G. With the PTA method, amplicons are preferentially generated from the primary template (“direct copies”) using a polymerase (e.g., a strand displacing polymerase). Consequently, errors are propagated at a lower rate from daughter amplicons during subsequent amplifications compared to MDA. The result is an easily executed method that, unlike existing WGA protocols, can amplify low DNA input including the genomes of single cells with high coverage breadth and uniformity in an accurate and reproducible manner. Moreover, the terminated amplification products can undergo direction ligation after removal of the terminators, allowing for the attachment of a cell barcode to the amplification primers so that products from all cells can be pooled after undergoing parallel amplification reactions. In some instances, template nucleic acids are not bound to a solid support. In some instances, direct copies of template nucleic acids are not bound to a solid support. In some instances, one or more primers are not bound to a solid support. In some instances, no primers are not bound to a solid support. In some instances, a primer is attached to a first solid support, and a template nucleic acid is attached to a second solid support, wherein the first and the second solid supports are not the same. In some instances, PTA is used to analyze single cells from a larger population of cells. In some instances, PTA is used to analyze more than one cell from a larger population of cells, or an entire population of cells.

Described herein are methods employing nucleic acid polymerases with strand displacement activity for amplification. In some instances, such polymerases comprise strand displacement activity and low error rate. In some instances, such polymerases comprise strand displacement activity and proofreading exonuclease activity, such as 3′->5′ proofreading activity. In some instances, nucleic acid polymerases are used in conjunction with other components such as reversible or irreversible terminators, or additional strand displacement factors. In some instances, the polymerase has strand displacement activity, but does not have exonuclease proofreading activity. For example, in some instances such polymerases include bacteriophage phi29 (Φ29) polymerase, which also has very low error rate that is the result of the 3′->5′ proofreading exonuclease activity (see, e.g., U.S. Pat. Nos. 5,198,543 and 5,001,050). In some instances, non-limiting examples of strand displacing nucleic acid polymerases include, e.g., genetically modified phi29 (Φ29) DNA polymerase, Klenow Fragment of DNA polymerase I (Jacobsen et al., Eur. J. Biochem. 45:623-627 (1974)), phage M2 DNA polymerase (Matsumoto et al., Gene 84:247 (1989)), phage phiPRD1 DNA polymerase (Jung et al., Proc. Natl. Acad. Sci. USA 84:8287 (1987); Zhu and Ito, Biochim. Biophys. Acta. 1219:267-276 (1994)), Bst DNA polymerase (e.g., Bst large fragment DNA polymerase (Exo(-) Bst; Aliotta et al., Genet. Anal. (Netherlands) 12:185-195 (1996)), exo(-)Bca DNA polymerase (Walker and Linn, Clinical Chemistry 42:1604-1608 (1996)), Bsu DNA polymerase, VentR DNA polymerase including VentR (exo-) DNA polymerase (Kong et al., J. Biol. Chem. 268:1965-1975 (1993)), Deep Vent DNA polymerase including Deep Vent (exo-) DNA polymerase, IsoPol DNA polymerase, DNA polymerase I, Therminator DNA polymerase, T5 DNA polymerase (Chatterjee et al., Gene 97:13-19 (1991)), Sequenase (U.S. Biochemicals), T7 DNA polymerase, T7-Sequenase, T7 gp5 DNA polymerase, PRDI DNA polymerase, T4 DNA polymerase (Kaboord and Benkovic, Curr. Biol. 5:149-157 (1995)). Additional strand displacing nucleic acid polymerases are also compatible with the methods described herein. The ability of a given polymerase to carry out strand displacement replication can be determined, for example, by using the polymerase in a strand displacement replication assay (e.g., as disclosed in U.S. Pat. No. 6,977,148). Such assays in some instances are performed at a temperature suitable for optimal activity for the enzyme being used, for example, 32° C. for phi29 DNA polymerase, from 46° C. to 64° C. for exo(-) Bst DNA polymerase, or from about 60° C. to 70° C. for an enzyme from a hyperthermophylic organism. Another useful assay for selecting a polymerase is the primer-block assay described in Kong et al., J. Biol. Chem. 268:1965-1975 (1993). The assay consists of a primer extension assay using an M13 ssDNA template in the presence or absence of an oligonucleotide that is hybridized upstream of the extending primer to block its progress. Other enzymes capable of displacement the blocking primer in this assay are in some instances useful for the disclosed method. In some instances, polymerases incorporate dNTPs and terminators at approximately equal rates. In some instances, the ratio of rates of incorporation for dNTPs and terminators for a polymerase described herein are about 1:1, about 1.5:1, about 2:1, about 3:1 about 4:1 about 5:1, about 10:1, about 20:1 about 50:1, about 100:1, about 200:1, about 500:1, or about 1000:1. In some instances, the ratio of rates of incorporation for dNTPs and terminators for a polymerase described herein are 1:1 to 1000:1, 2:1 to 500:1, 5:1 to 100:1, 10:1 to 1000:1, 100:1 to 1000:1, 500:1 to 2000:1, 50:1 to 1500:1, or 25:1 to 1000:1.

Described herein are methods of amplification wherein strand displacement can be facilitated through the use of a strand displacement factor, such as, e.g., helicase. Such factors are in some instances used in conjunction with additional amplification components, such as polymerases, terminators, or other component. In some instances, a strand displacement factor is used with a polymerase that does not have strand displacement activity. In some instances, a strand displacement factor is used with a polymerase having strand displacement activity. Without being bound by theory, strand displacement factors may increase the rate that smaller, double stranded amplicons are reprimed. In some instances, any DNA polymerase that can perform strand displacement replication in the presence of a strand displacement factor is suitable for use in the PTA method, even if the DNA polymerase does not perform strand displacement replication in the absence of such a factor. Strand displacement factors useful in strand displacement replication in some instances include (but are not limited to) BMRF1 polymerase accessory subunit (Tsurumi et al., J. Virology 67(12):7648-7653 (1993)), adenovirus DNA-binding protein (Zijderveld and van der Vliet, J. Virology 68(2): 1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996);T7 helicase-primase; T7 gp2.5 SSB protein; Tte-UvrD (from Thermoanaerobacter tengcongensis), calf thymus helicase (Siegel et al., J. Biol. Chem. 267:13629-13635 (1992)); bacterial SSB (e.g., E. coli SSB), Replication Protein A (RPA) in eukaryotes, human mitochondrial SSB (mtSSB), and recombinases, (e.g., Recombinase A (RecA) family proteins, T4 UvsX, T4 UvsY, Sak4 of Phage HK620, Rad51, Dmc1, or Radb). Combinations of factors that facilitate strand displacement and priming are also consistent with the methods described herein. For example, a helicase is used in conjunction with a polymerase. In some instances, the PTA method comprises use of a single-strand DNA binding protein (SSB, T4 gp32, or other single stranded DNA binding protein), a helicase, and a polymerase (e.g., SauDNA polymerase, Bsu polymerase, Bst2.0, GspM, GspM2.0, GspSSD, or other suitable polymerase). In some instances, reverse transcriptases are used in conjunction with the strand displacement factors described herein. In some instances, reverse transcriptases are used in conjunction with the strand displacement factors described herein. In some instances, amplification is conducted using a polymerase and a nicking enzyme (e.g., “NEAR”), such as those described in U.S. Pat. No. 9,617,586. In some instances, the nicking enzyme is Nt.BspQI, Nb.BbvCi, Nb.Bsml, Nb.BsrDI, Nb.Btsl, Nt.AlwI, Nt.BbvCI, Nt.BstNBI, Nt.CviPII, Nb.Bpu10I, or Nt.Bpu10I.

Described herein are amplification methods comprising use of terminator nucleotides, polymerases, and additional factors or conditions. For example, such factors are used in some instances to fragment the nucleic acid template(s) or amplicons during amplification. In some instances, such factors comprise endonucleases. In some instances, factors comprise transposases. In some instances, mechanical shearing is used to fragment nucleic acids during amplification. In some instances, nucleotides are added during amplification that may be fragmented through the addition of additional proteins or conditions. For example, uracil is incorporated into amplicons; treatment with uracil D-glycosylase fragments nucleic acids at uracil-containing positions. Additional systems for selective nucleic acid fragmentation are also in some instances employed, for example an engineered DNA glycosylase that cleaves modified cytosine-pyrene base pairs. (Kwon, et al. Chem Biol. 2003, 10(4), 351)

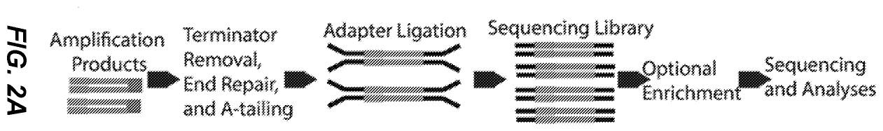

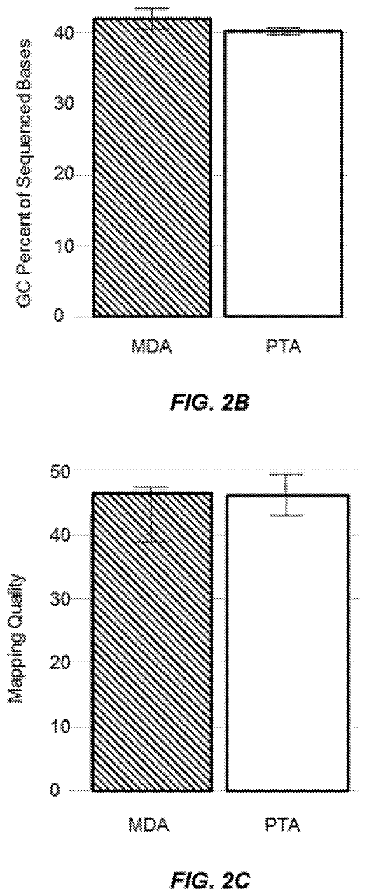

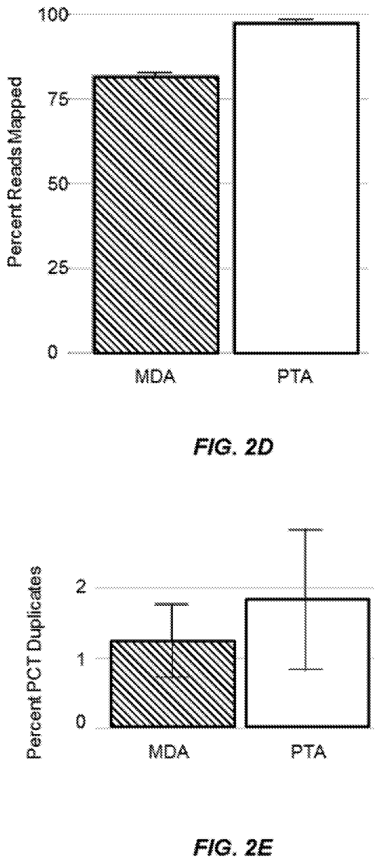

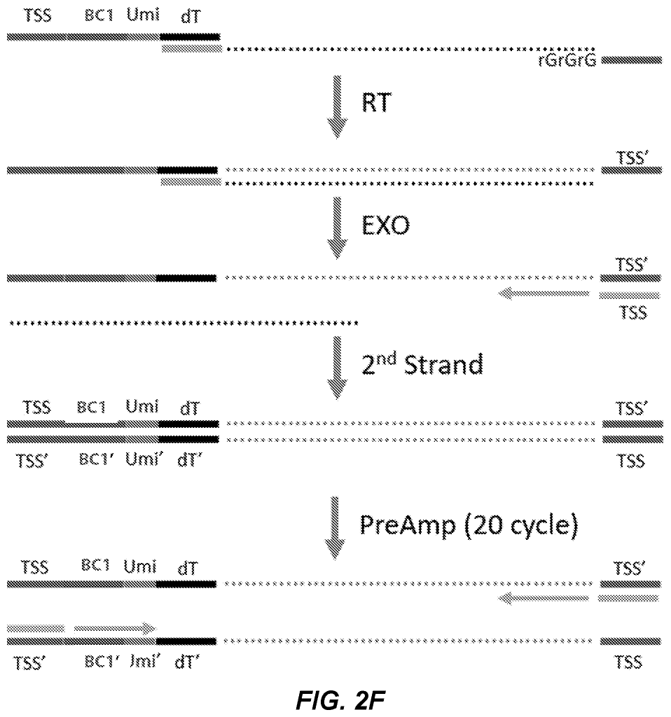

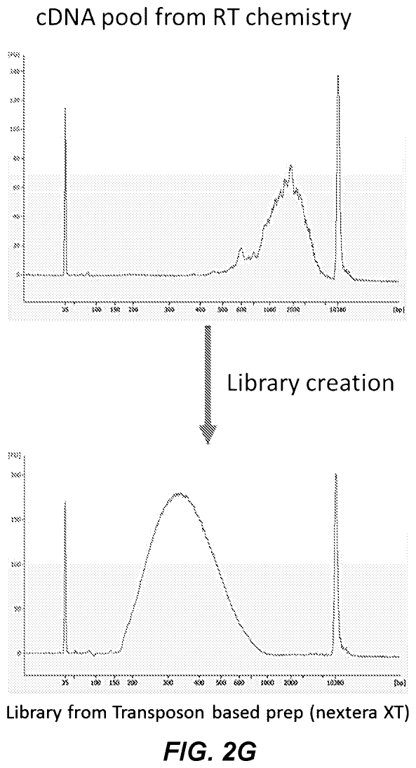

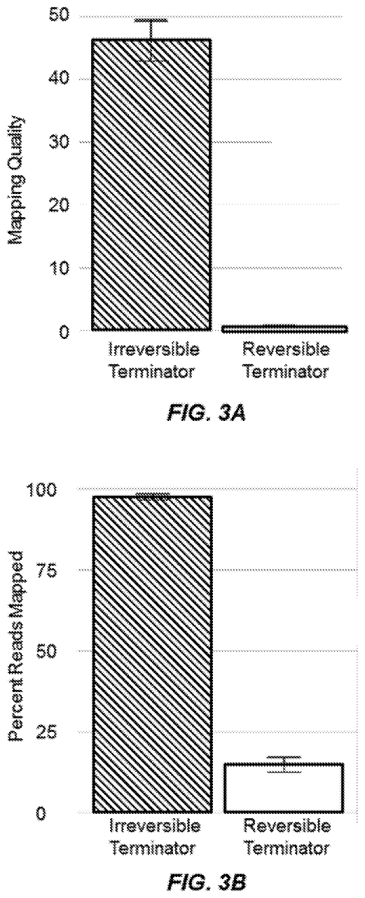

Described herein are amplification methods comprising use of terminator nucleotides, which terminate nucleic acid replication thus decreasing the size of the amplification products. Such terminators are in some instances used in conjunction with polymerases, strand displacement factors, or other amplification components described herein. In some instances, terminator nucleotides reduce or lower the efficiency of nucleic acid replication. Such terminators in some instances reduce extension rates by at least 99.9%, 99%, 98%, 95%, 90%, 85%, 80%, 75%, 70%, or at least 65%. Such terminators in some instances reduce extension rates by 50%-90%, 60%-80%, 65%-90%, 70%-85%, 60%-90%, 70%-99%, 80%-99%, or 50%-80%. In some instances terminators reduce the average amplicon product length by at least 99.9%, 99%, 98%, 95%, 90%, 85%, 80%, 75%, 70%, or at least 65%. Terminators in some instances reduce the average amplicon length by 50%-90%, 60%-80%, 65%-90%, 70%-85%, 60%-90%, 70%-99%, 80%-99%, or 50%-80%. In some instances, amplicons comprising terminator nucleotides form loops or hairpins which reduce a polymerase's ability to use such amplicons as templates. Use of terminators in some instances slows the rate of amplification at initial amplification sites through the incorporation of terminator nucleotides (e.g., dideoxynucleotides that have been modified to make them exonuclease-resistant to terminate DNA extension), resulting in smaller amplification products. By producing smaller amplification products than the currently used methods (e.g., average length of 50-2000 nucleotides in length for PTA methods as compared to an average product length of >10,000 nucleotides for MDA methods) PTA amplification products in some instances undergo direct ligation of adapters without the need for fragmentation, allowing for efficient incorporation of cell barcodes and unique molecular identifiers (UMI) (see FIG. 2A).

Terminator nucleotides are present at various concentrations depending on factors such as polymerase, template, or other factors. For example, the amount of terminator nucleotides in some instances is expressed as a ratio of non-terminator nucleotides to terminator nucleotides in a method described herein. Such concentrations in some instances allow control of amplicon lengths. In some instances, the ratio of terminator to non-terminator nucleotides is modified for the amount of template present or the size of the template. In some instances, the ratio of ratio of terminator to non-terminator nucleotides is reduced for smaller samples sizes (e.g., femtogram to picogram range). In some instances, the ratio of non-terminator to terminator nucleotides is about 2:1, 5:1, 7:1, 10:1, 20:1, 50:1, 100:1, 200:1, 500:1, 1000:1, 2000:1, or 5000:1. In some instances the ratio of non-terminator to terminator nucleotides is 2:1-10:1, 5:1-20:1, 10:1-100:1, 20:1-200:1, 50:1-1000:1, 50:1-500:1, 75:1-150:1, or 100:1-500:1. In some instances, at least one of the nucleotides present during amplification using a method described herein is a terminator nucleotide. Each terminator need not be present at approximately the same concentration; in some instances, ratios of each terminator present in a method described herein are optimized for a particular set of reaction conditions, sample type, or polymerase. Without being bound by theory, each terminator may possess a different efficiency for incorporation into the growing polynucleotide chain of an amplicon, in response to pairing with the corresponding nucleotide on the template strand. For example, in some instances a terminator pairing with cytosine is present at about 3%, 5%, 10%, 15%, 20%, 25%, or 50% higher concentration than the average terminator concentration. In some instances a terminator pairing with thymine is present at about 3%, 5%, 10%, 15%, 20%, 25%, or 50% higher concentration than the average terminator concentration. In some instances a terminator pairing with guanine is present at about 3%, 5%, 10%, 15%, 20%, 25%, or 50% higher concentration than the average terminator concentration. In some instances a terminator pairing with adenine is present at about 3%, 5%, 10%, 15%, 20%, 25%, or 50% higher concentration than the average terminator concentration. In some instances a terminator pairing with uracil is present at about 3%, 5%, 10%, 15%, 20%, 25%, or 50% higher concentration than the average terminator concentration. Any nucleotide capable of terminating nucleic acid extension by a nucleic acid polymerase in some instances is used as a terminator nucleotide in the methods described herein. In some instances, a reversible terminator is used to terminate nucleic acid replication. In some instances, a non-reversible terminator is used to terminate nucleic acid replication. In some instances, non-limited examples of terminators include reversible and non-reversible nucleic acids and nucleic acid analogs, such as, e.g., 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, or any combination thereof. In one embodiment, terminator nucleotides are dideoxynucleotides. Other nucleotide modifications that terminate nucleic acid replication and may be suitable for practicing the invention include, without limitation, any modifications of the r group of the 3′ carbon of the deoxyribose such as inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, terminators are polynucleotides comprising 1, 2, 3, 4, or more bases in length. In some instances, terminators do not comprise a detectable moiety or tag (e.g., mass tag, fluorescent tag, dye, radioactive atom, or other detectable moiety). In some instances, terminators do not comprise a chemical moiety allowing for attachment of a detectable moiety or tag (e.g., “click” azide/alkyne, conjugate addition partner, or other chemical handle for attachment of a tag). In some instances, all terminator nucleotides comprise the same modification that reduces amplification to at region (e.g., the sugar moiety, base moiety, or phosphate moiety) of the nucleotide. In some instances, at least one terminator has a different modification that reduces amplification. In some instances, all terminators have a substantially similar fluorescent excitation or emission wavelengths. In some instances, terminators without modification to the phosphate group are used with polymerases that do not have exonuclease proofreading activity. Terminators, when used with polymerases which have 3′->5′ proofreading exonuclease activity (such as, e.g., phi29) that can remove the terminator nucleotide, are in some instances further modified to make them exonuclease-resistant. For example, dideoxynucleotides are modified with an alpha-thio group that creates a phosphorothioate linkage which makes these nucleotides resistant to the 3′->5′ proofreading exonuclease activity of nucleic acid polymerases. Such modifications in some instances reduce the exonuclease proofreading activity of polymerases by at least 99.5%, 99%, 98%, 95%, 90%, or at least 85%. Non-limiting examples of other terminator nucleotide modifications providing resistance to the 3′->5′ exonuclease activity include in some instances: nucleotides with modification to the alpha group, such as alpha-thio dideoxynucleotides creating a phosphorothioate bond, C3 spacer nucleotides, locked nucleic acids (LNA), inverted nucleic acids, 2′ Fluoro bases, 3′ phosphorylation, 2′-O-Methyl modifications (or other 2′-O-alkyl modification), propyne-modified bases (e.g., deoxycytosine, deoxyuridine), L-DNA nucleotides, L-RNA nucleotides, nucleotides with inverted linkages (e.g., 5′-5′ or 3′-3′), 5′ inverted bases (e.g., 5′ inverted 2′,3′-dideoxy dT), methylphosphonate backbones, and trans nucleic acids. In some instances, nucleotides with modification include base-modified nucleic acids comprising free 3′ OH groups (e.g., 2-nitrobenzyl alkylated HOMedU triphosphates, bases comprising modification with large chemical groups, such as solid supports or other large moiety). In some instances, a polymerase with strand displacement activity but without 3′->5′exonuclease proofreading activity is used with terminator nucleotides with or without modifications to make them exonuclease resistant. Such nucleic acid polymerases include, without limitation, Bst DNA polymerase, Bsu DNA polymerase, Deep Vent (exo-) DNA polymerase, Klenow Fragment (exo-) DNA polymerase, Therminator DNA polymerase, and VentR (exo-).

Primers and Amplicon Libraries

Described herein are amplicon libraries resulting from amplification of at least one target nucleic acid molecule. Such libraries are in some instances generated using the methods described herein, such as those using terminators. Such methods comprise use of strand displacement polymerases or factors, terminator nucleotides (reversible or irreversible), or other features and embodiments described herein. In some instances, amplicon libraries generated by use of terminators described herein are further amplified in a subsequent amplification reaction (e.g., PCR). In some instances, subsequent amplification reactions do not comprise terminators. In some instances, amplicon libraries comprise polynucleotides, wherein at least 50%, 60%, 70%, 80%, 90%, 95%, or at least 98% of the polynucleotides comprise at least one terminator nucleotide. In some instances, the amplicon library comprises the target nucleic acid molecule from which the amplicon library was derived. The amplicon library comprises a plurality of polynucleotides, wherein at least some of the polynucleotides are direct copies (e.g., replicated directly from a target nucleic acid molecule, such as genomic DNA, RNA, or other target nucleic acid). For example, at least 5%, 10%, 20%, 30%, 40%, 50%, 60%, 70%, 80%, 90%, 95% or more than 95% of the amplicon polynucleotides are direct copies of the at least one target nucleic acid molecule. In some instances, at least 5% of the amplicon polynucleotides are direct copies of the at least one target nucleic acid molecule. In some instances, at least 10% of the amplicon polynucleotides are direct copies of the at least one target nucleic acid molecule. In some instances, at least 15% of the amplicon polynucleotides are direct copies of the at least one target nucleic acid molecule. In some instances, at least 20% of the amplicon polynucleotides are direct copies of the at least one target nucleic acid molecule. In some instances, at least 50% of the amplicon polynucleotides are direct copies of the at least one target nucleic acid molecule. In some instances, 3%-5%, 3-10%, 5%-10%, 10%-20%, 20%-30%, 30%-40%, 5%-30%, 10%-50%, or 15%-75% of the amplicon polynucleotides are direct copies of the at least one target nucleic acid molecule. In some instances, at least some of the polynucleotides are direct copies of the target nucleic acid molecule, or daughter (a first copy of the target nucleic acid) progeny. For example, at least 5%, 10%, 20%, 30%, 40%, 50%, 60%, 70%, 80%, 90%, 95% or more than 95% of the amplicon polynucleotides are direct copies of the at least one target nucleic acid molecule or daughter progeny. In some instances, at least 5% of the amplicon polynucleotides are direct copies of the at least one target nucleic acid molecule or daughter progeny. In some instances, at least 10% of the amplicon polynucleotides are direct copies of the at least one target nucleic acid molecule or daughter progeny. In some instances, at least 20% of the amplicon polynucleotides are direct copies of the at least one target nucleic acid molecule or daughter progeny. In some instances, at least 30% of the amplicon polynucleotides are direct copies of the at least one target nucleic acid molecule or daughter progeny. In some instances, 3%-5%, 3%-10%, 5%-10%, 10%-20%, 20%-30%, 30%-40%, 5%-30%, 10%-50%, or 15%-75% of the amplicon polynucleotides are direct copies of the at least one target nucleic acid molecule or daughter progeny. In some instances, direct copies of the target nucleic acid are 50-2500, 75-2000, 50-2000, 25-1000, 50-1000, 500-2000, or 50-2000 bases in length. In some instances, daughter progeny are 1000-5000, 2000-5000, 1000-10,000, 2000-5000, 1500-5000, 3000-7000, or 2000-7000 bases in length. In some instances, the average length of PTA amplification products is 25-3000 nucleotides in length, 50-2500, 75-2000, 50-2000, 25-1000, 50-1000, 500-2000, or 50-2000 bases in length. In some instance, amplicons generated from PTA are no more than 5000, 4000, 3000, 2000, 1700, 1500, 1200, 1000, 700, 500, or no more than 300 bases in length. In some instance, amplicons generated from PTA are 1000-5000, 1000-3000, 200-2000, 200-4000, 500-2000, 750-2500, or 1000-2000 bases in length. Amplicon libraries generated using the methods described herein in some instances comprise at least 1000, 2000, 5000, 10,000, 100,000, 200,000, 500,000 or more than 500,000 amplicons comprising unique sequences. In some instances, the library comprises at least 100, 200, 300, 400, 500, 600, 700, 800, 900, 1000, 1100, 1200, 1300, 1400, 1500, 2000, 2500, 3000, or at least 3500 amplicons. In some instances, at least 5%, 10%, 15%, 20%, 25%, 30% or more than 30% of amplicon polynucleotides having a length of less than 1000 bases are direct copies of the at least one target nucleic acid molecule. In some instances, at least 5%, 10%, 15%, 20%, 25%, 30% or more than 30% of amplicon polynucleotides having a length of no more than 2000 bases are direct copies of the at least one target nucleic acid molecule. In some instances, at least 5%, 10%, 15%, 20%, 25%, 30% or more than 30% of amplicon polynucleotides having a length of 3000-5000 bases are direct copies of the at least one target nucleic acid molecule. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are no more than 700-1200 bases in length. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are 700-1200 bases in length, and the daughter amplicons are 2500-6000 bases in length. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule or daughter amplicons. The number of direct copies may be controlled in some instances by the number of PCR amplification cycles. In some instances, no more than 30, 25, 20, 15, 13, 11, 10, 9, 8, 7, 6, 5, 4, or 3 PCR cycles are used to generate copies of the target nucleic acid molecule. In some instances, about 30, 25, 20, 15, 13, 11, 10, 9, 8, 7, 6, 5, 4, or about 3 PCR cycles are used to generate copies of the target nucleic acid molecule. In some instances, 3, 4, 5, 6, 7, or 8 PCR cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 PCR cycles are used to generate copies of the target nucleic acid molecule. Amplicon libraries generated using the methods described herein are in some instances subjected to additional steps, such as adapter ligation and further PCR amplification. In some instances, such additional steps precede a sequencing step.

Methods described herein may additionally comprise one or more enrichment or purification steps. In some instances, one or more polynucleotides (such as cDNA, PTA amplicons, or other polynucleotide) are enriched during a method described herein. In some instances, polynucleotide probes are used to capture one or more polynucleotides. In some instances, probes are configured to capture one or more genomic exons. In some instances, a library of probes comprises at least 1000, 2000, 5000, 10,000, 50,000, 100,000, 200,000, 500,000, or more than 1 million different sequences. In some instances, a library of probes comprises sequences capable of binding to at least 10, 20, 50, 100, 200, 500, 1000, 2000, 5000, 10,000 or more than 10,000 genes. In some instances, probes comprise a moiety for capture by a solid support, such as biotin. In some instances, an enrichment step occurs after a PTA step. In some instances, an enrichment step occurs before a PTA step. In some instances, probes are configured to bind genomic DNA libraries. In some instances, probes are configured to bind cDNA libraries.

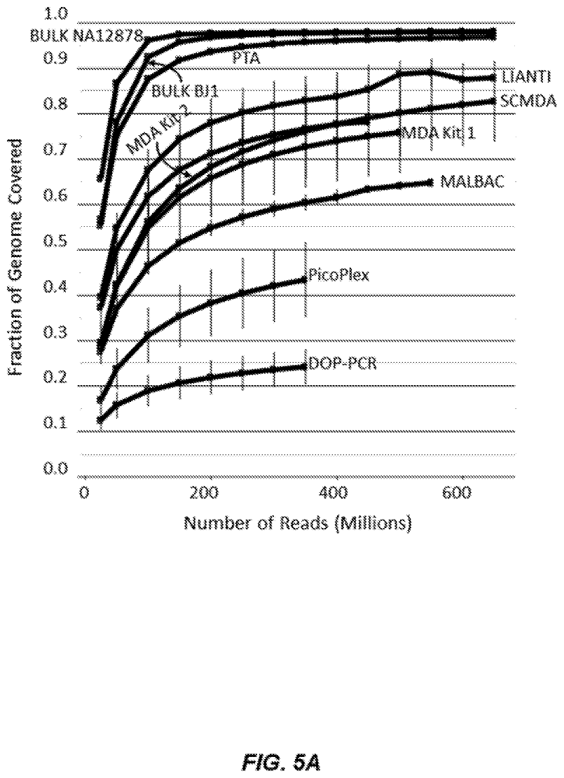

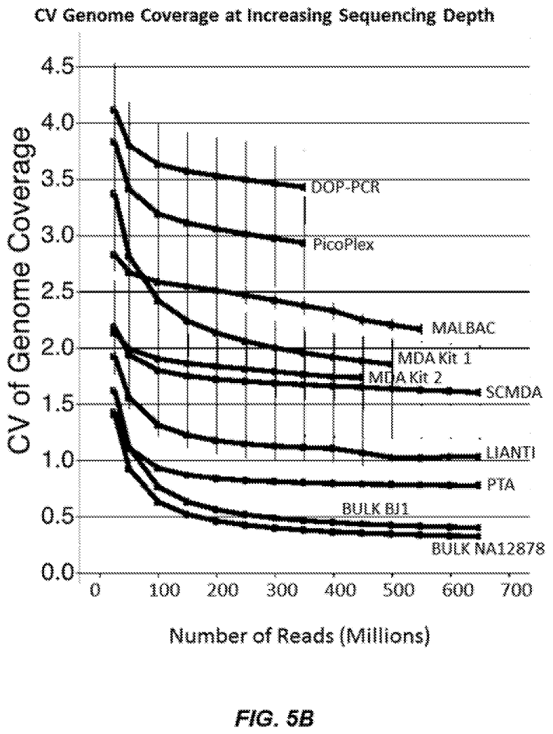

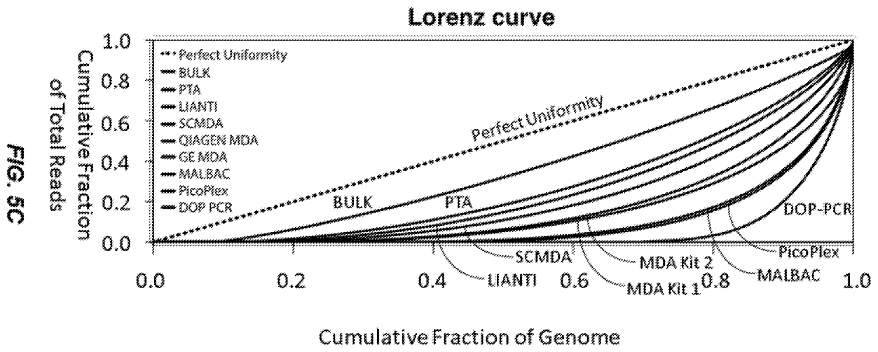

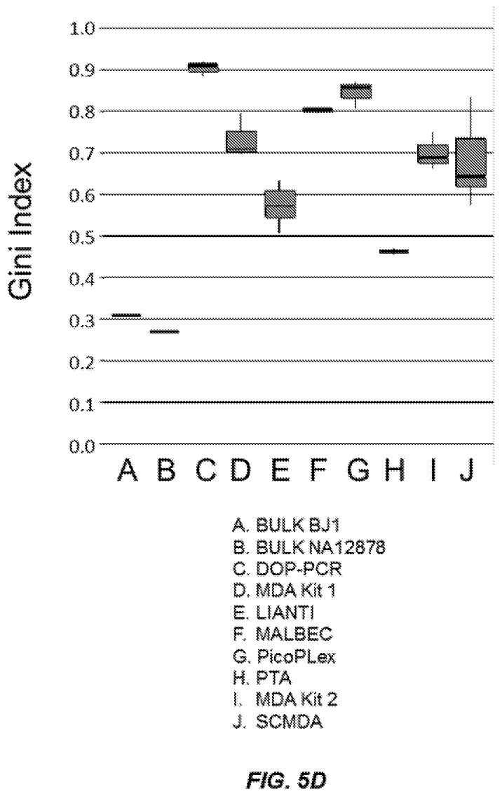

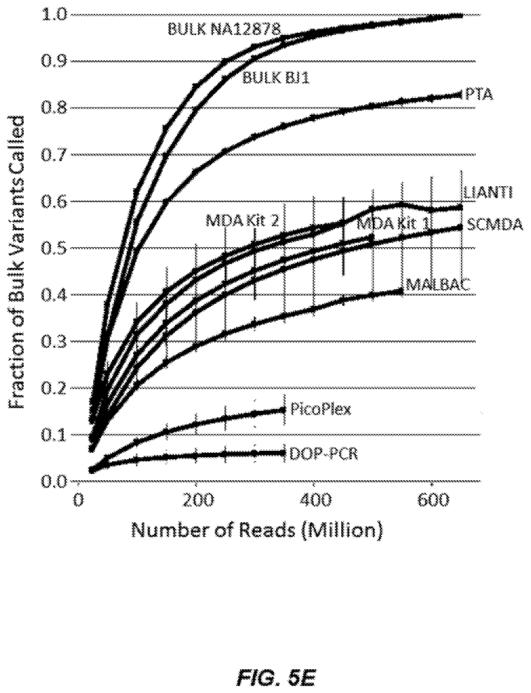

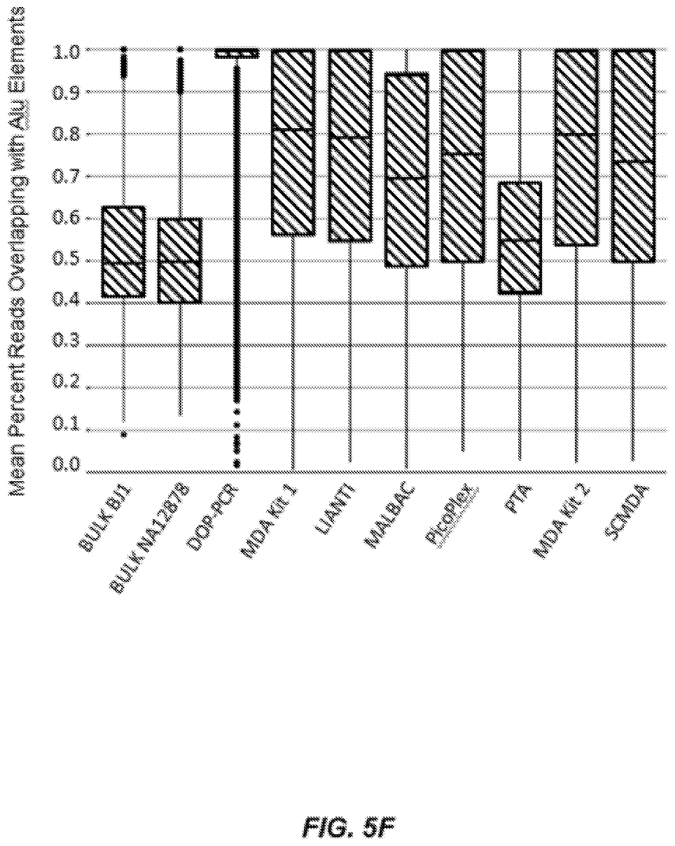

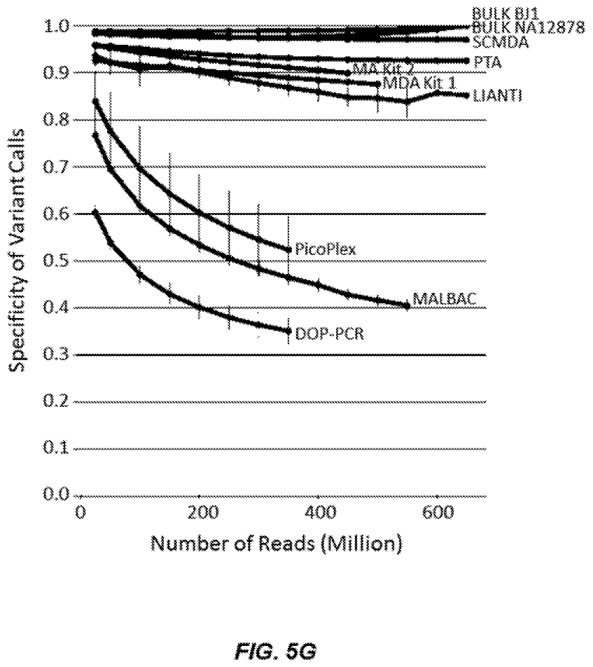

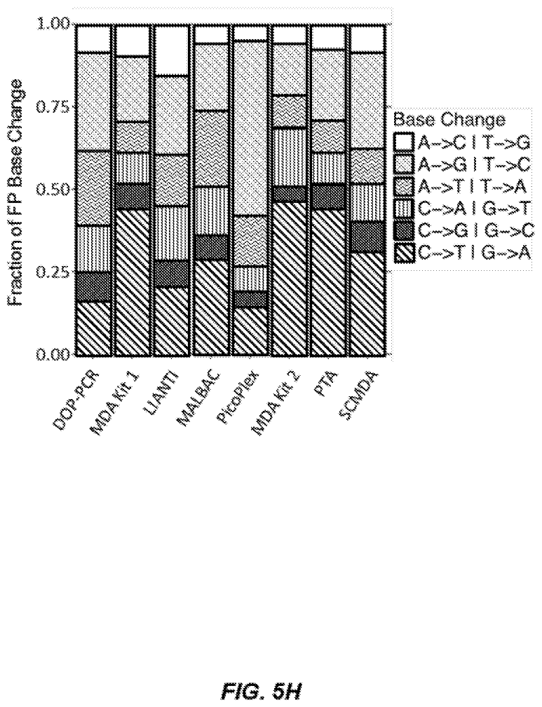

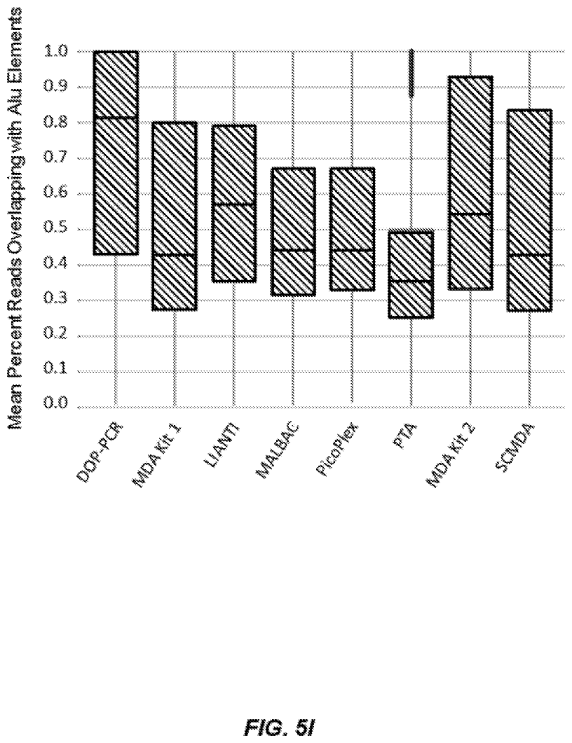

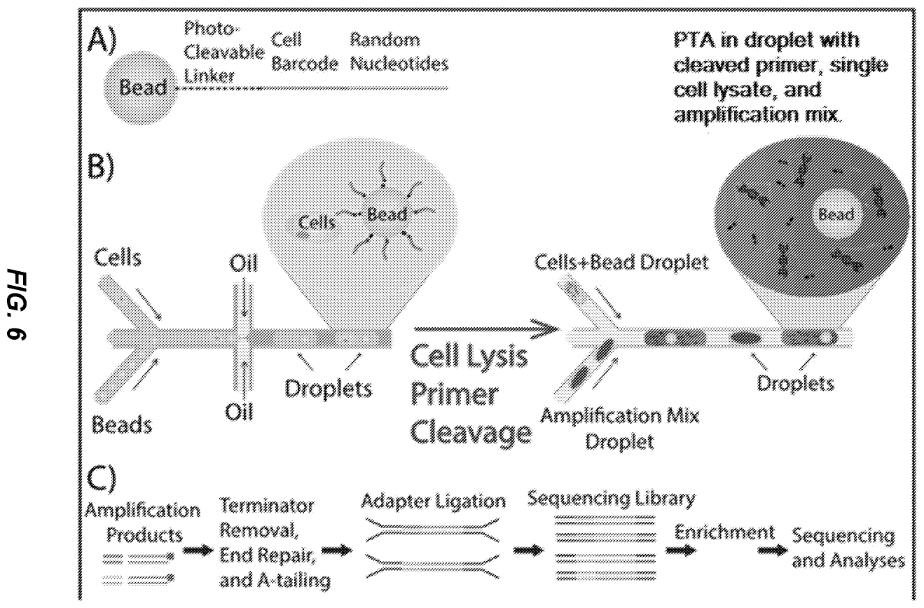

Amplicon libraries of polynucleotides generated from the PTA methods and compositions (terminators, polymerases, etc.) described herein in some instances have increased uniformity. Uniformity, in some instances, is described using a Lorenz curve (e.g., FIG. 5C), or other such method. Such increases in some instances lead to lower sequencing reads needed for the desired coverage of a target nucleic acid molecule (e.g., genomic DNA, RNA, or other target nucleic acid molecule). For example, no more than 50% of a cumulative fraction of polynucleotides comprises sequences of at least 80% of a cumulative fraction of sequences of the target nucleic acid molecule. In some instances, no more than 50% of a cumulative fraction of polynucleotides comprises sequences of at least 60% of a cumulative fraction of sequences of the target nucleic acid molecule. In some instances, no more than 50% of a cumulative fraction of polynucleotides comprises sequences of at least 70% of a cumulative fraction of sequences of the target nucleic acid molecule. In some instances, no more than 50% of a cumulative fraction of polynucleotides comprises sequences of at least 90% of a cumulative fraction of sequences of the target nucleic acid molecule. In some instances, uniformity is described using a Gini index (wherein an index of 0 represents perfect equality of the library and an index of 1 represents perfect inequality). In some instances, amplicon libraries described herein have a Gini index of no more than 0.55, 0.50, 0.45, 0.40, or 0.30. In some instances, amplicon libraries described herein have a Gini index of no more than 0.50. In some instances, amplicon libraries described herein have a Gini index of no more than 0.40. Such uniformity metrics in some instances are dependent on the number of reads obtained. For example, no more than 100 million, 200 million, 300 million, 400 million, or no more than 500 million reads are obtained. In some instances, the read length is about 50, 75, 100, 125, 150, 175, 200, 225, or about 250 bases in length. In some instances, uniformity metrics are dependent on the depth of coverage of a target nucleic acid. For example, the average depth of coverage is about 10×, 15×, 20×, 25×, or about 30×. In some instances, the average depth of coverage is 10-30×, 20-50×, 5-40×, 20-60×, 5-20×, or 10-20×. In some instances, amplicon libraries described herein have a Gini index of no more than 0.55, wherein about 300 million reads was obtained. In some instances, amplicon libraries described herein have a Gini index of no more than 0.50, wherein about 300 million reads was obtained. In some instances, amplicon libraries described herein have a Gini index of no more than 0.45, wherein about 300 million reads was obtained. In some instances, amplicon libraries described herein have a Gini index of no more than 0.55, wherein no more than 300 million reads was obtained. In some instances, amplicon libraries described herein have a Gini index of no more than 0.50, wherein no more than 300 million reads was obtained. In some instances, amplicon libraries described herein have a Gini index of no more than 0.45, wherein no more than 300 million reads was obtained. In some instances, amplicon libraries described herein have a Gini index of no more than 0.55, wherein the average depth of sequencing coverage is about 15×. In some instances, amplicon libraries described herein have a Gini index of no more than 0.50, wherein the average depth of sequencing coverage is about 15×. In some instances, amplicon libraries described herein have a Gini index of no more than 0.45, wherein the average depth of sequencing coverage is about 15×. In some instances, amplicon libraries described herein have a Gini index of no more than 0.55, wherein the average depth of sequencing coverage is at least 15×. In some instances, amplicon libraries described herein have a Gini index of no more than 0.50, wherein the average depth of sequencing coverage is at least 15×. In some instances, amplicon libraries described herein have a Gini index of no more than 0.45, wherein the average depth of sequencing coverage is at least 15×. In some instances, amplicon libraries described herein have a Gini index of no more than 0.55, wherein the average depth of sequencing coverage is no more than 15×. In some instances, amplicon libraries described herein have a Gini index of no more than 0.50, wherein the average depth of sequencing coverage is no more than 15×. In some instances, amplicon libraries described herein have a Gini index of no more than 0.45, wherein the average depth of sequencing coverage is no more than 15×. Uniform amplicon libraries generated using the methods described herein are in some instances subjected to additional steps, such as adapter ligation and further PCR amplification. In some instances, such additional steps precede a sequencing step.

Primers comprise nucleic acids used for priming the amplification reactions described herein. Such primers in some instances include, without limitation, random deoxynucleotides of any length with or without modifications to make them exonuclease resistant, random ribonucleotides of any length with or without modifications to make them exonuclease resistant, modified nucleic acids such as locked nucleic acids, DNA or RNA primers that are targeted to a specific genomic region, and reactions that are primed with enzymes such as primase. In the case of whole genome PTA, it is preferred that a set of primers having random or partially random nucleotide sequences be used. In a nucleic acid sample of significant complexity, specific nucleic acid sequences present in the sample need not be known and the primers need not be designed to be complementary to any particular sequence. Rather, the complexity of the nucleic acid sample results in a large number of different hybridization target sequences in the sample, which will be complementary to various primers of random or partially random sequence. The complementary portion of primers for use in PTA are in some instances fully randomized, comprise only a portion that is randomized, or be otherwise selectively randomized. The number of random base positions in the complementary portion of primers in some instances, for example, is from 20% to 100% of the total number of nucleotides in the complementary portion of the primers. In some instances, the number of random base positions in the complementary portion of primers is 10% to 90%, 15-95%, 20%-100%, 30%-100%, 50%-100%, 75-100% or 90-95% of the total number of nucleotides in the complementary portion of the primers. In some instances, the number of random base positions in the complementary portion of primers is at least 10%, 20%, 30%, 40%, 50%, 60%, 70%, 80%, or at least 90% of the total number of nucleotides in the complementary portion of the primers. Sets of primers having random or partially random sequences are in some instances synthesized using standard techniques by allowing the addition of any nucleotide at each position to be randomized. In some instances, sets of primers are composed of primers of similar length and/or hybridization characteristics. In some instances, the term “random primer” refers to a primer which can exhibit four-fold degeneracy at each position. In some instances, the term “random primer” refers to a primer which can exhibit three-fold degeneracy at each position. Random primers used in the methods described herein in some instances comprise a random sequence that is 3, 4, 5, 6, 7, 8, 10, 11, 12, 13, 14, 15, 16, 17, 18, 19, 20, or more bases in length. In some instances, primers comprise random sequences that are 3-20, 5-15, 5-20, 6-12, or 4-10 bases in length. Primers may also comprise non-extendable elements that limit subsequent amplification of amplicons generated thereof. For example, primers with non-extendable elements in some instances comprise terminators. In some instances, primers comprise terminator nucleotides, such as 1, 2, 3, 4, 5, 10, or more than 10 terminator nucleotides. Primers need not be limited to components which are added externally to an amplification reaction. In some instances, primers are generated in-situ through the addition of nucleotides and proteins which promote priming. For example, primase-like enzymes in combination with nucleotides is in some instances used to generate random primers for the methods described herein. Primase-like enzymes in some instances are members of the DnaG or AEP enzyme superfamily. In some instances, a primase-like enzyme is TthPrimPol. In some instances, a primase-like enzyme is T7 gp4 helicase-primase. Such primases are in some instances used with the polymerases or strand displacement factors described herein. In some instances, primases initiate priming with deoxyribonucleotides. In some instances, primases initiate priming with ribonucleotides.

The PTA amplification can be followed by selection for a specific subset of amplicons. Such selections are in some instances dependent on size, affinity, activity, hybridization to probes, or other known selection factor in the art. In some instances, selections precede or follow additional steps described herein, such as adapter ligation and/or library amplification. In some instances, selections are based on size (length) of the amplicons. In some instances, smaller amplicons are selected that are less likely to have undergone exponential amplification, which enriches for products that were derived from the primary template while further converting the amplification from an exponential into a quasi-linear amplification process (FIG. 1A). In some instances, amplicons comprising 50-2000, 25-5000, 40-3000, 50-1000, 200-1000, 300-1000, 400-1000, 400-600, 600-2000, or 800-1000 bases in length are selected. Size selection in some instances occurs with the use of protocols, e.g., utilizing solid-phase reversible immobilization (SPRI) on carboxylated paramagnetic beads to enrich for nucleic acid fragments of specific sizes, or other protocol known by those skilled in the art. Optionally or in combination, selection occurs through preferential ligation and amplification of smaller fragments during PCR while preparing sequencing libraries, as well as a result of the preferential formation of clusters from smaller sequencing library fragments during sequencing (e.g., sequencing by synthesis, nanopore sequencing, or other sequencing method). Other strategies to select for smaller fragments are also consistent with the methods described herein and include, without limitation, isolating nucleic acid fragments of specific sizes after gel electrophoresis, the use of silica columns that bind nucleic acid fragments of specific sizes, and the use of other PCR strategies that more strongly enrich for smaller fragments. Any number of library preparation protocols may be used with the PTA methods described herein. Amplicons generated by PTA are in some instances ligated to adapters (optionally with removal of terminator nucleotides). In some instances, amplicons generated by PTA comprise regions of homology generated from transposase-based fragmentation which are used as priming sites. In some instances, libraries are prepared by fragmenting nucleic acids mechanically or enzymatically. In some instances, libraries are prepared using tagmentation via transposomes. In some instances, libraries are prepared via ligation of adapters, such as Y-adapters, universal adapters, or circular adapters.

The non-complementary portion of a primer used in PTA can include sequences which can be used to further manipulate and/or analyze amplified sequences. An example of such a sequence is a “detection tag”. Detection tags have sequences complementary to detection probes and are detected using their cognate detection probes. There may be one, two, three, four, or more than four detection tags on a primer. There is no fundamental limit to the number of detection tags that can be present on a primer except the size of the primer. In some instances, there is a single detection tag on a primer. In some instances, there are two detection tags on a primer. When there are multiple detection tags, they may have the same sequence or they may have different sequences, with each different sequence complementary to a different detection probe. In some instances, multiple detection tags have the same sequence. In some instances, multiple detection tags have a different sequence.

Another example of a sequence that can be included in the non-complementary portion of a primer is an “address tag” that can encode other details of the amplicons, such as the location in a tissue section. In some instances, a cell barcode comprises an address tag. An address tag has a sequence complementary to an address probe. Address tags become incorporated at the ends of amplified strands. If present, there may be one, or more than one, address tag on a primer. There is no fundamental limit to the number of address tags that can be present on a primer except the size of the primer. When there are multiple address tags, they may have the same sequence or they may have different sequences, with each different sequence complementary to a different address probe. The address tag portion can be any length that supports specific and stable hybridization between the address tag and the address probe. In some instances, nucleic acids from more than one source can incorporate a variable tag sequence. This tag sequence can be up to 100 nucleotides in length, preferably 1 to 10 nucleotides in length, most preferably 4, 5 or 6 nucleotides in length and comprises combinations of nucleotides. In some instances, a tag sequence is 1-20, 2-15, 3-13, 4-12, 5-12, or 1-10 nucleotides in length For example, if six base-pairs are chosen to form the tag and a permutation of four different nucleotides is used, then a total of 4096 nucleic acid anchors (e.g. hairpins), each with a unique 6 base tag can be made.

Primers described herein may be present in solution or immobilized on a solid support. In some instances, primers bearing sample barcodes and/or UMI sequences can be immobilized on a solid support. The solid support can be, for example, one or more beads. In some instances, individual cells are contacted with one or more beads having a unique set of sample barcodes and/or UMI sequences in order to identify the individual cell. In some instances, lysates from individual cells are contacted with one or more beads having a unique set of sample barcodes and/or UMI sequences in order to identify the individual cell lysates. In some instances, extracted nucleic acid from individual cells are contacted with one or more beads having a unique set of sample barcodes and/or UMI sequences in order to identify the extracted nucleic acid from the individual cell. The beads can be manipulated in any suitable manner as is known in the art, for example, using droplet actuators as described herein. The beads may be any suitable size, including for example, microbeads, microparticles, nanobeads and nanoparticles. In some embodiments, beads are magnetically responsive; in other embodiments beads are not significantly magnetically responsive. Non-limiting examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Pat. Appl. Pub. No. US20050260686, US20030132538, US20050118574, 20050277197, 20060159962. Beads may be pre-coupled with an antibody, protein or antigen, DNA/RNA probe or any other molecule with an affinity for a desired target. In some embodiments, primers bearing sample barcodes and/or UMI sequences can be in solution. In certain embodiments, a plurality of droplets can be presented, wherein each droplet in the plurality bears a sample barcode which is unique to a droplet and the UMI which is unique to a molecule such that the UMI are repeated many times within a collection of droplets. In some embodiments, individual cells are contacted with a droplet having a unique set of sample barcodes and/or UMI sequences in order to identify the individual cell. In some embodiments, lysates from individual cells are contacted with a droplet having a unique set of sample barcodes and/or UMI sequences in order to identify the individual cell lysates. In some embodiments, extracted nucleic acid from individual cells are contacted with a droplet having a unique set of sample barcodes and/or UMI sequences in order to identify the extracted nucleic acid from the individual cell.

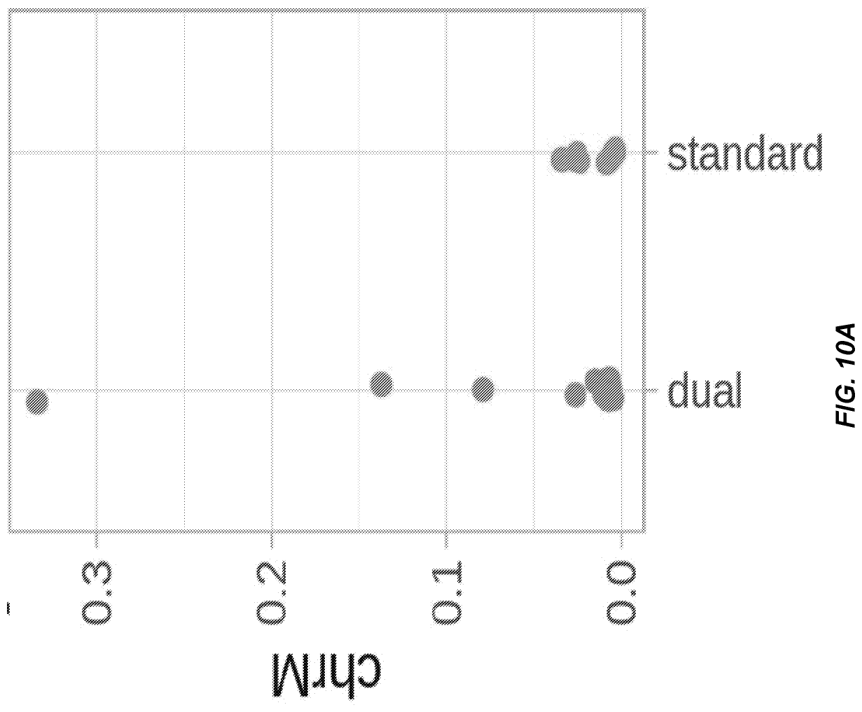

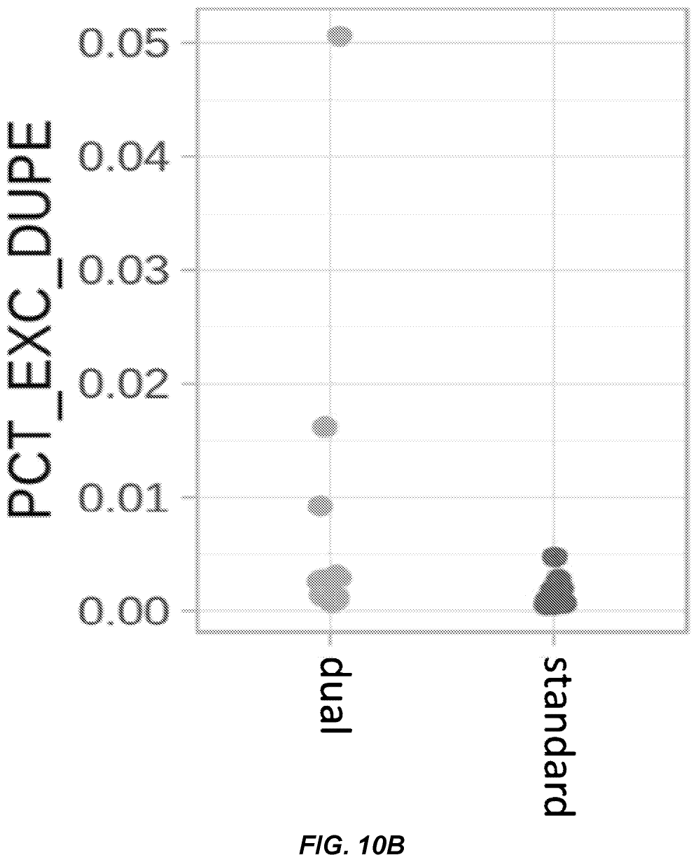

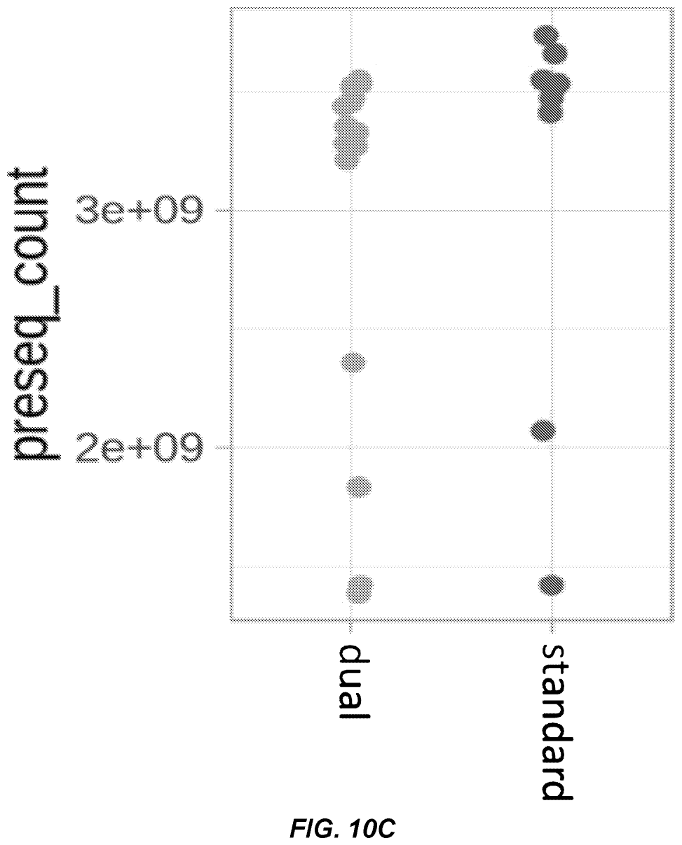

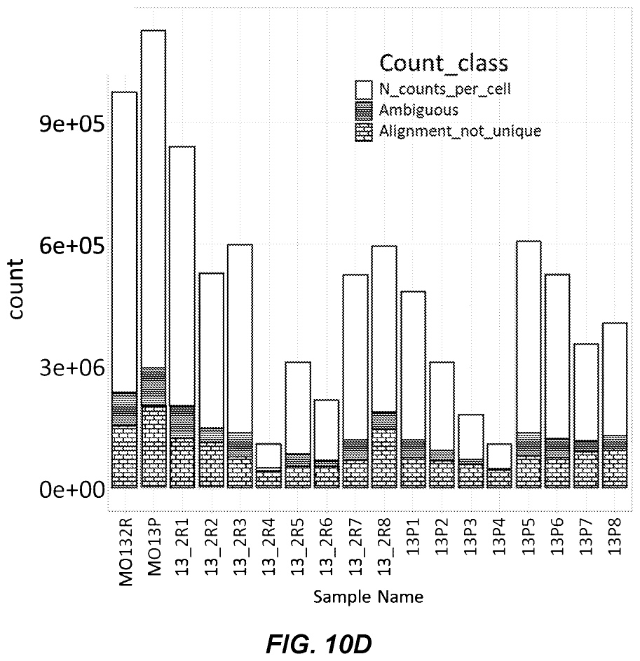

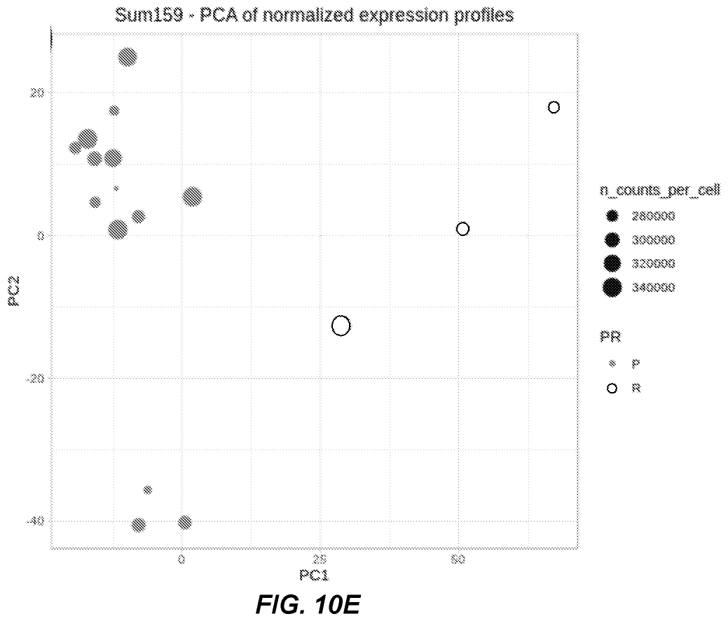

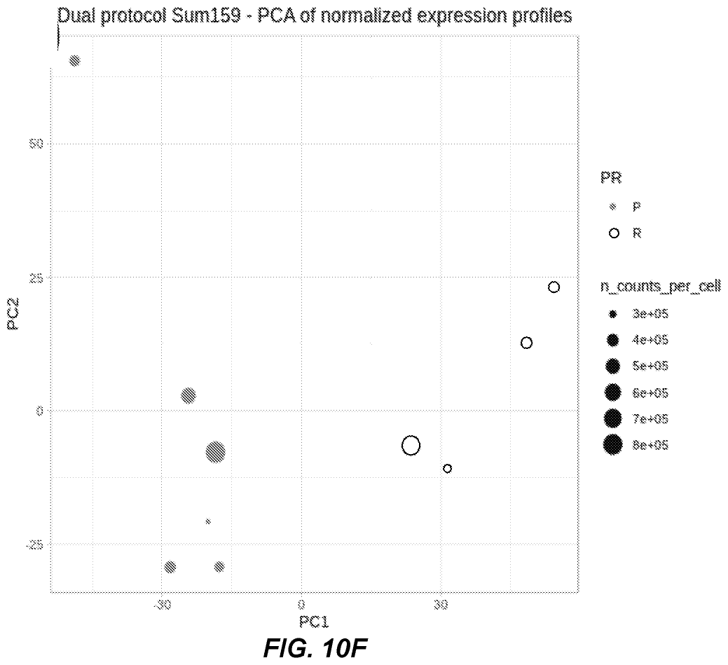

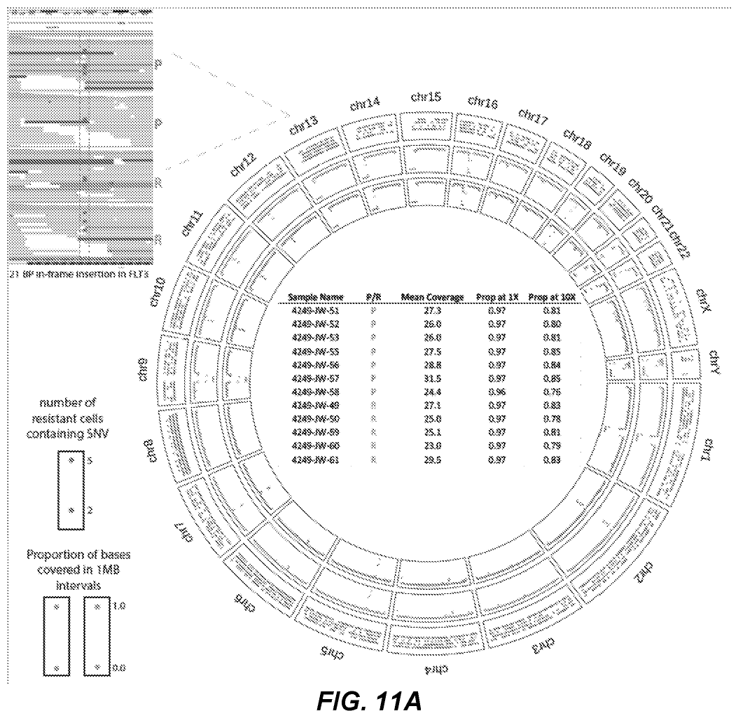

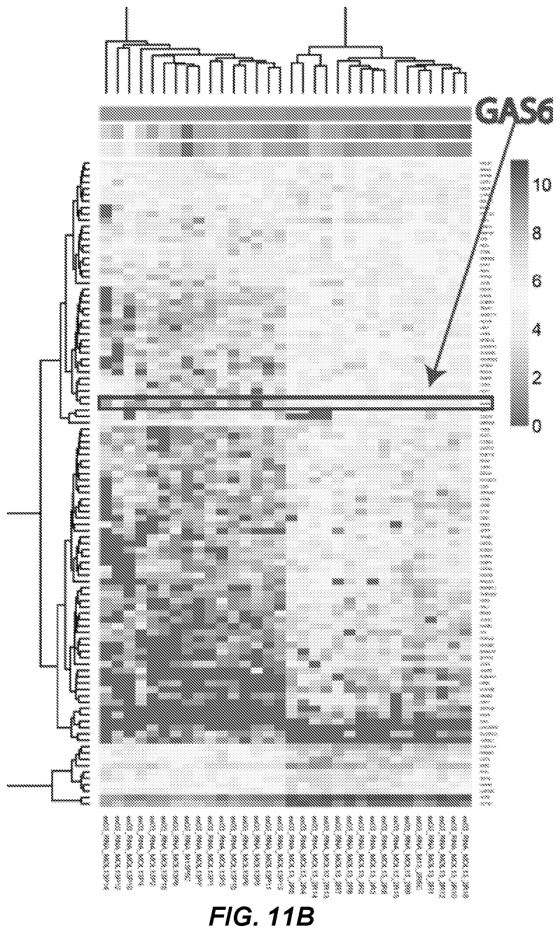

PTA primers may comprise a sequence-specific or random primer, a cell barcode and/or a unique molecular identifier (UMI) (see, e.g., FIGS. 10A (linear primer) and 10B (hairpin primer)). In some instances, a primer comprises a sequence-specific primer. In some instances, a primer comprises a random primer. In some instances, a primer comprises a cell barcode. In some instances, a primer comprises a sample barcode. In some instances, a primer comprises a unique molecular identifier. In some instances, primers comprise two or more cell barcodes. Such barcodes in some instances identify a unique sample source, or unique workflow. Such barcodes or UMIs are in some instances 5, 6, 7, 8, 9, 10, 11, 12, 15, 20, 25, 30, or more than 30 bases in length. Primers in some instances comprise at least 1000, 10,000, 50,000, 100,000, 250,000, 500,000, 10⁶, 10⁷, 10⁸, 10⁹, or at least 10¹⁰ unique barcodes or UMIs. In some instances primers comprise at least 8, 16, 96, or 384 unique barcodes or UMIs. In some instances a standard adapter is then ligated onto the amplification products prior to sequencing; after sequencing, reads are first assigned to a specific cell based on the cell barcode. Suitable adapters that may be utilized with the PTA method include, e.g., xGen® Dual Index UMI adapters available from Integrated DNA Technologies (IDT). Reads from each cell is then grouped using the UMI, and reads with the same UMI may be collapsed into a consensus read. The use of a cell barcode allows all cells to be pooled prior to library preparation, as they can later be identified by the cell barcode. The use of the UMI to form a consensus read in some instances corrects for PCR bias, improving the copy number variation (CNV) detection (FIGS. 11A and 11B). In addition, sequencing errors may be corrected by requiring that a fixed percentage of reads from the same molecule have the same base change detected at each position. This approach has been utilized to improve CNV detection and correct sequencing errors in bulk samples. In some instances, UMIs are used with the methods described herein, for example, U.S Pat. No. 8,835,358 discloses the principle of digital counting after attaching a random amplifiable barcode. Schmitt. et al and Fan et al. disclose similar methods of correcting sequencing errors. In some instances, a library is generated for sequencing using primers. In some instances, the library comprises fragments of 200-700 bases, 100-1000, 300-800, 300-550, 300-700, or 200-800 bases in length. In some instances, the library comprises fragments of at least 50, 100, 150, 200, 300, 500, 600, 700, 800, or at least 1000 bases in length. In some instances, the library comprises fragments of about 50, 100, 150, 200, 300, 500, 600, 700, 800, or about 1000 bases in length.

The methods described herein may further comprise additional steps, including steps performed on the sample or template. Such samples or templates in some instance are subjected to one or more steps prior to PTA. In some instances, samples comprising cells are subjected to a pre-treatment step. For example, cells undergo lysis and proteolysis to increase chromatin accessibility using a combination of freeze-thawing, Triton X-100, Tween 20, and Proteinase K. Other lysis strategies are also be suitable for practicing the methods described herein. Such strategies include, without limitation, lysis using other combinations of detergent and/or lysozyme and/or protease treatment and/or physical disruption of cells such as sonication and/or alkaline lysis and/or hypotonic lysis. In some instances, the primary template or target molecule(s) is subjected to a pre-treatment step. In some instances, the primary template (or target) is denatured using sodium hydroxide, followed by neutralization of the solution. Other denaturing strategies may also be suitable for practicing the methods described herein. Such strategies may include, without limitation, combinations of alkaline lysis with other basic solutions, increasing the temperature of the sample and/or altering the salt concentration in the sample, addition of additives such as solvents or oils, other modification, or any combination thereof. In some instances, additional steps include sorting, filtering, or isolating samples, templates, or amplicons by size. In some instances, cells are lysed with mechanical (e.g., high pressure homogenizer, bead milling) or non-mechanical (physical, chemical, or biological). In some instances, physical lysis methods comprise heating, osmotic shock, and/or cavitation. In some instances, chemical lysis comprises alkali and/or detergents. In some instances, biological lysis comprises use of enzymes. Combinations of lysis methods are also compatible with the methods described herein. Non-limited examples of lysis enzymes include recombinant lysozyme, serine proteases, and bacterial lysins. In some instances, lysis with enzymes comprises use of lysozyme, lysostaphin, zymolase, cellulose, protease or glycanase. For example, after amplification with the methods described herein, amplicon libraries are enriched for amplicons having a desired length. In some instances, amplicon libraries are enriched for amplicons having a length of 50-2000, 25-1000, 50-1000, 75-2000, 100-3000, 150-500, 75-250, 170-500, 100-500, or 75-2000 bases. In some instances, amplicon libraries are enriched for amplicons having a length no more than 75, 100, 150, 200, 500, 750, 1000, 2000, 5000, or no more than 10,000 bases. In some instances, amplicon libraries are enriched for amplicons having a length of at least 25, 50, 75, 100, 150, 200, 500, 750, 1000, or at least 2000 bases.

Methods and compositions described herein may comprise buffers or other formulations. Such buffers are in some instances used for PTA, RT, or other method described herein. Such buffers in some instances comprise surfactants/detergent or denaturing agents (Tween-20, DMSO, DMF, pegylated polymers comprising a hydrophobic group, or other surfactant), salts (potassium or sodium phosphate (monobasic or dibasic), sodium chloride, potassium chloride, TrisHCl, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, buffers are used in conjunction with components such as polymerases, strand displacement factors, terminators, or other reaction component described herein. In some instances, buffers are used in conjunction with components such as polymerases, strand displacement factors, terminators, or other reaction component described herein. Buffers may comprise one or more crowding agents. In some instances, crowding reagents include polymers. In some instances, crowding reagents comprise polymers such as polyols. In some instances, crowding reagents comprise polyethylene glycol polymers (PEG). In some instances, crowding reagents comprise polysaccharides. Without limitation, examples of crowding reagents include ficoll (e.g., ficoll PM 400, ficoll PM 70, or other molecular weight ficoll), PEG (e.g., PEG1000, PEG 2000, PEG4000, PEG6000, PEG8000, or other molecular weight PEG), dextran (dextran 6, dextran 10, dextran 40, dextran 70, dextran 6000, dextran 138k, or other molecular weight dextran).

The nucleic acid molecules amplified according to the methods described herein may be sequenced and analyzed using methods known to those of skill in the art. Non-limiting examples of the sequencing methods which in some instances are used include, e.g., sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (Int. Pat. Appl. Pub. No. WO2006/073504), multiplex sequencing (U.S. Pat. Appl. Pub. No. US2008/0269068; Porreca et al., 2007, Nat. Methods 4:931), polymerized colony (POLONY) sequencing (U.S. Pat. Nos. 6,432,360, 6,485,944 and 6,511,803, and Int. Pat. Appl. Pub. No. WO2005/082098), nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout), high-throughput sequencing methods such as, e.g., methods using Roche 454, Illumina Solexa, AB-SOLiD, Helicos, Polonator platforms and the like, and light-based sequencing technologies (Landegren et al. (1998) Genome Res. 8:769-76; Kwok (2000) Pharmacogenomics 1:95-100; and Shi (2001) Clin. Chem.47:164-172). In some instances, the amplified nucleic acid molecules are shotgun sequenced. Sequencing of the sequencing library is in some instances performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis (array/colony-based or nanoball based).

Sequencing libraries generated using the methods described herein (e.g., PTA or RNAseq) may be sequenced to obtain a desired number of sequencing reads. In some instances, libraries are generated from a single cell or sample comprising a single cell (alone or part of a multiomics workflow). In some instances, libraries are sequenced to obtain at least 0.1, 0.2, 0.4, 0.5, 0.7, 0.8, 0.9, 1, 1.1, 1.2, 1.5, 2, 5, or at least 10 million reads. In some instances, libraries are sequenced to obtain no more than 0.1, 0.2, 0.4, 0.5, 0.7, 0.8, 0.9, 1, 1.1, 1.2, 1.5, 2, 5, or no more than 10 million reads. In some instances, libraries are sequenced to obtain about 0.1, 0.2, 0.4, 0.5, 0.7, 0.8, 0.9, 1, 1.1, 1.2, 1.5, 2, 5, or about 10 million reads. In some instances, libraries are sequenced to obtain 0.1-10, 0.1-5, 0.1-1, 0.2-1, 0.3-1.5, 0.5-1, 1-5, or 0.5-5 million reads per sample. In some instances, the number of reads is dependent on the size of the genome. In some in instances samples comprising bacterial genomes are sequenced to obtain 0.5-1 million reads. In some instances, libraries are sequenced to obtain at least 2, 4, 10, 20, 50, 100, 200, 300, 500, 700, or at least 900 million reads. In some instances, libraries are sequenced to obtain no more than 2, 4, 10, 20, 50, 100, 200, 300, 500, 700, or no more than 900 million reads. In some instances, libraries are sequenced to obtain about 2, 4, 10, 20, 50, 100, 200, 300, 500, 700, or about 900 million reads. In some in instances samples comprising mammalian genomes are sequenced to obtain 500-600 million reads. In some instances, the type of sequencing library (cDNA libraries or genomic libraries) are identified during sequencing. In some instances, cDNA libraries and genomic libraries are identified during sequencing with unique barcodes.

The term “cycle” when used in reference to a polymerase-mediated amplification reaction is used herein to describe steps of dissociation of at least a portion of a double stranded nucleic acid (e.g., a template from an amplicon, or a double stranded template, denaturation). hybridization of at least a portion of a primer to a template (annealing), and extension of the primer to generate an amplicon. In some instances, the temperature remains constant during a cycle of amplification (e.g., an isothermal reaction). In some instances, the number of cycles is directly correlated with the number of amplicons produced. In some instances, the number of cycles for an isothermal reaction is controlled by the amount of time the reaction is allowed to proceed.

Methods and Applications

Described herein are methods of identifying mutations in cells with the methods of multiomic analysis PTA, such as single cells. Use of the PTA method in some instances results in improvements over known methods, for example, MDA. PTA in some instances has lower false positive and false negative variant calling rates than the MDA method. Genomes, such as NA12878 platinum genomes, are in some instances used to determine if the greater genome coverage and uniformity of PTA would result in lower false negative variant calling rate. Without being bound by theory, it may be determined that the lack of error propagation in PTA decreases the false positive variant call rate. The amplification balance between alleles with the two methods is in some cases estimated by comparing the allele frequencies of the heterozygous mutation calls at known positive loci. In some instances, amplicon libraries generated using PTA are further amplified by PCR. In some instances, PTA is used in a workflow with additional analysis methods, such as RNAseq, methylome analysis or other method described herein.

Cells analyzed using the methods described herein in some instances comprise tumor cells. For example, circulating tumor cells can be isolated from a fluid taken from patients, such as but not limited to, blood, bone marrow, urine, saliva, cerebrospinal fluid, pleural fluid, pericardial fluid, ascites, or aqueous humor. The cells are then subjected to the methods described herein (e.g. PTA) and sequencing to determine mutation burden and mutation combination in each cell. These data are in some instances used for the diagnosis of a specific disease or as tools to predict treatment response. Similarly, in some instances cells of unknown malignant potential in some instances are isolated from fluid taken from patients, such as but not limited to, blood, bone marrow, urine, saliva, cerebrospinal fluid, pleural fluid, pericardial fluid, ascites, aqueous humor, blastocoel fluid, or collection media surrounding cells in culture. In some instances, a sample is obtained from collection media surrounding embryonic cells. After utilizing the methods described herein and sequencing, such methods are further used to determine mutation burden and mutation combination in each cell. These data are in some instances used for the diagnosis of a specific disease or as tools to predict progression of a premalignant state to overt malignancy. In some instances, cells can be isolated from primary tumor samples. The cells can then undergo PTA and sequencing to determine mutation burden and mutation combination in each cell. These data can be used for the diagnosis of a specific disease or are as tools to predict the probability that a patient's malignancy is resistant to available anti-cancer drugs. By exposing samples to different chemotherapy agents, it has been found that the major and minor clones have differential sensitivity to specific drugs that does not necessarily correlate with the presence of a known “driver mutation,” suggesting that combinations of mutations within a clonal population determine its sensitivities to specific chemotherapy drugs. Without being bound by theory, these findings suggest that a malignancy may be easier to eradicate if premalignant lesions that have not yet expanded are and evolved into clones are detected whose increased number of genome modification may make them more likely to be resistant to treatment. See, Ma et al., 2018, “Pan-cancer genome and transcriptome analyses of 1,699 pediatric leukemias and solid tumors.” A single-cell genomics protocol is in some instances used to detect the combinations of somatic genetic variants in a single cancer cell, or clonotype, within a mixture of normal and malignant cells that are isolated from patient samples. This technology is in some instances further utilized to identify clonotypes that undergo positive selection after exposure to drugs, both in vitro and/or in patients. As shown in FIG. 6A, by comparing the surviving clones exposed to chemotherapy compared to the clones identified at diagnosis, a catalog of cancer clonotypes can be created that documents their resistance to specific drugs. PTA methods in some instances detect the sensitivity of specific clones in a sample composed of multiple clonotypes to existing or novel drugs, as well as combinations thereof, where the method can detect the sensitivity of specific clones to the drug. This approach in some instances shows efficacy of a drug for a specific clone that may not be detected with current drug sensitivity measurements that consider the sensitivity of all cancer clones together in one measurement. When the PTA described herein are applied to patient samples collected at the time of diagnosis in order to detect the cancer clonotypes in a given patient's cancer, a catalog of drug sensitivities may then be used to look up those clones and thereby inform oncologists as to which drug or combination of drugs will not work and which drug or combination of drugs is most likely to be efficacious against that patient's cancer. The PTA may be used for analysis of samples comprising groups of cells. In some instances, a sample comprises neurons or glial cells. In some instances, the sample comprises nuclei.

Described herein are methods of measuring the gene expression alteration in combination with the mutagenicity of an environmental factor. For example, cells (single or a population) are exposed to a potential environmental condition. For example, cells such originating from organs (liver, pancreas, lung, colon, thyroid, or other organ), tissues (skin, or other tissue), blood, or other biological source are in some instances used with the method. In some instances, an environmental condition comprises heat, light (e.g. ultraviolet), radiation, a chemical substance, or any combination thereof. After an amount of exposure to the environmental condition, in some instances minutes, hours, days, or longer, single cells are isolated and subjected to the PTA method. In some instances, molecular barcodes and unique molecular identifiers are used to tag the sample. The sample is sequenced and then analyzed to identify gene expression alterations and or resulting from mutations resulting from exposure to the environmental condition. In some instances, such mutations are compared with a control environmental condition, such as a known non-mutagenic substance, vehicle/solvent, or lack of an environmental condition. Such analysis in some instances not only provides the total number of mutations caused by the environmental condition, but also the locations and nature of such mutations. Patterns are in some instances identified from the data, and may be used for diagnosis of diseases or conditions. In some instances, patterns are used to predict future disease states or conditions. In some instances, the methods described herein measure the mutation burden, locations, and patterns in a cell after exposure to an environmental agent, such as, e.g., a potential mutagen or teratogen. This approach in some instances is used to evaluate the safety of a given agent, including its potential to induce mutations that can contribute to the development of a disease. For example, the method could be used to predict the carcinogenicity or teratogenicity of an agent to specific cell types after exposure to a specific concentration of the specific agent.

Described herein are methods of identifying gene expression alteration in combination with the mutations in animal, plant or microbial cells that have undergone genome editing (e.g., using CRISPR technologies). Such cells in some instances can be isolated and subjected to PTA and sequencing to determine mutation burden and mutation combination in each cell. The per-cell mutation rate and locations of mutations that result from a genome editing protocol are in some instances used to assess the safety of a given genome editing method.

Described herein are methods of determining gene expression alteration in combination with the mutations in cells that are used for cellular therapy, such as but not limited to the transplantation of induced pluripotent stem cells, transplantation of hematopoietic or other cells that have not be manipulated, or transplantation of hematopoietic or other cells that have undergone genome edits. The cells can then undergo PTA and sequencing to determine mutation burden and mutation combination in each cell. The per-cell mutation rate and locations of mutations in the cellular therapy product can be used to assess the safety and potential efficacy of the product.

Cells for use with the PTA method may be fetal cells, such as embryonic cells. In some embodiments, PTA is used in conjunction with non-invasive preimplantation genetic testing (NIPGT). In a further embodiment, cells can be isolated from blastomeres that are created by in vitro fertilization. The cells can then undergo PTA and sequencing to determine the burden and combination of potentially disease predisposing genetic variants in each cell. The gene expression alteration in combination with the mutation profile of the cell can then be used to extrapolate the genetic predisposition of the blastomere to specific diseases prior to implantation. In some instances embryos in culture shed nucleic acids that are used to assess the health of the embryo using low pass genome sequencing. In some instances, embryos are frozen-thawed. In some instances, nucleic acids are obtained from blastocyte culture conditioned medium (BCCM), blastocoel fluid (BF), or a combination thereof. In some instances, PTA analysis of fetal cells is used to detect chromosomal abnormalities, such as fetal aneploidy. In some instances, PTA is used to detect diseases such as Down's or Patau syndromes. In some instances, frozen blastocytes are thawed and cultured for a period of time before obtaining nucleic acids for analysis (e.g., culture media, BF, or a cell biopsy). In some instances, blastocytes are cultured for no more than 4, 6, 8, 12, 16, 24, 36, 48, or no more than 64 hours prior to obtaining nucleic acids for analysis.

In another embodiment, microbial cells (e.g., bacteria, fungi, protozoa) can be isolated from plants or animals (e.g., from microbiota samples [e.g., GI microbiota, skin microbiota, etc.] or from bodily fluids such as, e.g., blood, bone marrow, urine, saliva, cerebrospinal fluid, pleural fluid, pericardial fluid, ascites, or aqueous humor). In addition, microbial cells may be isolated from indwelling medical devices, such as but not limited to, intravenous catheters, urethral catheters, cerebrospinal shunts, prosthetic valves, artificial joints, or endotracheal tubes. The cells can then undergo PTA and sequencing to determine the identity of a specific microbe, as well as to detect the presence of microbial genetic variants that predict response (or resistance) to specific antimicrobial agents. These data can be used for the diagnosis of a specific infectious disease and/or as tools to predict treatment response.

Described herein are methods generating amplicon libraries from samples comprising short nucleic acid using the PTA methods described herein. In some instances, PTA leads to improved fidelity and uniformity of amplification of shorter nucleic acids. In some instances, nucleic acids are no more than 2000 bases in length. In some instances, nucleic acids are no more than 1000 bases in length. In some instances, nucleic acids are no more than 500 bases in length. In some instances, nucleic acids are no more than 200, 400, 750, 1000, 2000 or 5000 bases in length. In some instances, samples comprising short nucleic acid fragments include but at not limited to ancient DNA (hundreds, thousands, millions, or even billions of years old), FFPE (Formalin-Fixed Paraffin-Embedded) samples, cell-free DNA, or other sample comprising short nucleic acids.

### Embodiments

Described herein are methods of amplifying a target nucleic acid molecule, the method comprising: a) bringing into contact a sample comprising the target nucleic acid molecule, one or more amplification primers, a nucleic acid polymerase, and a mixture of nucleotides which comprises one or more terminator nucleotides which terminate nucleic acid replication by the polymerase, and b) incubating the sample under conditions that promote replication of the target nucleic acid molecule to obtain a plurality of terminated amplification products, wherein the replication proceeds by strand displacement replication. In one embodiment of any of the above methods, the method further comprises isolating from the plurality of terminated amplification products the products which are between about 50 and about 2000 nucleotides in length. In one embodiment of any of the above methods, the method further comprises isolating from the plurality of terminated amplification products the products which are between about 400 and about 600 nucleotides in length. In one embodiment of any of the above methods, the method further comprises: c) repairing ends and A-tailing, and d) ligating the molecules obtained in step (c) to adaptors, and thereby generating a library of amplification products. In some embodiments, the method further comprises removal of the terminator nucleotides from the terminated amplification products. In one embodiment of any of the above methods, the method further comprises sequencing the amplification products. In one embodiment of any of the above methods, the amplification is performed under substantially isothermic conditions. In one embodiment of any of the above methods, the nucleic acid polymerase is a DNA polymerase.

In one embodiment of any of the above methods, the DNA polymerase is a strand displacing DNA polymerase. In one embodiment of any of the above methods, the nucleic acid polymerase is selected from bacteriophage phi29 (129) polymerase, genetically modified phi29 (129) DNA polymerase, Klenow Fragment of DNA polymerase I, phage M2 DNA polymerase, phage phiPRD1 DNA polymerase, Bst DNA polymerase, Bst large fragment DNA polymerase, exo(-) Bst polymerase, exo(-)Bca DNA polymerase, Bsu DNA polymerase, VentR DNA polymerase, VentR (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of any of the above methods, the nucleic acid polymerase has 3′->5′ exonuclease activity and the terminator nucleotides inhibit such 3′->5′ exonuclease activity. In one specific embodiment, the terminator nucleotides are selected from nucleotides with modification to the alpha group (e.g., alpha-thio dideoxynucleotides creating a phosphorothioate bond), C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. In one embodiment of any of the above methods, the nucleic acid polymerase does not have 3′->5′ exonuclease activity. In one specific embodiment, the polymerase is selected from Bst DNA polymerase, exo(-) Bst polymerase, exo(-) Bca DNA polymerase, Bsu DNA polymerase, VentR (exo-) DNA polymerase, Deep Vent (exo-) DNA polymerase, Klenow Fragment (exo-) DNA polymerase, and Therminator DNA polymerase. In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In one embodiment of any of the above methods, the amplification primers are between 4 and 70 nucleotides long. In one embodiment of any of the above methods, the amplification products are between about 50 and about 2000 nucleotides in length. In one embodiment of any of the above methods, the target nucleic acid is DNA (e.g., a cDNA or a genomic DNA). In one embodiment of any of the above methods, the amplification primers are random primers. In one embodiment of any of the above methods, the amplification primers comprise a barcode. In one specific embodiment, the barcode comprises a cell barcode. In one specific embodiment, the barcode comprises a sample barcode. In one embodiment of any of the above methods, the amplification primers comprise a unique molecular identifier (UMI). In one embodiment of any of the above methods, the method comprises denaturing the target nucleic acid or genomic DNA before the initial primer annealing. In one specific embodiment, denaturation is conducted under alkaline conditions followed by neutralization. In one embodiment of any of the above methods, the sample, the amplification primers, the nucleic acid polymerase, and the mixture of nucleotides are contained in a microfluidic device. In one embodiment of any of the above methods, the sample, the amplification primers, the nucleic acid polymerase, and the mixture of nucleotides are contained in a droplet. In one embodiment of any of the above methods, the sample is selected from tissue(s) samples, cells, biological fluid samples (e.g., blood, urine, saliva, lymphatic fluid, cerebrospinal fluid (CSF), amniotic fluid, pleural fluid, pericardial fluid, ascites, aqueous humor), bone marrow samples, semen samples, biopsy samples, cancer samples, tumor samples, cell lysate samples, forensic samples, archaeological samples, paleontological samples, infection samples, production samples, whole plants, plant parts, microbiota samples, viral preparations, soil samples, marine samples, freshwater samples, household or industrial samples, and combinations and isolates thereof. In one embodiment of any of the above methods, the sample is a cell (e.g., an animal cell [e.g., a human cell], a plant cell, a fungal cell, a bacterial cell, and a protozoal cell). In one specific embodiment, the cell is lysed prior to the replication. In one specific embodiment, cell lysis is accompanied by proteolysis. In one specific embodiment, the cell is selected from a cell from a preimplantation embryo, a stem cell, a fetal cell, a tumor cell, a suspected cancer cell, a cancer cell, a cell subjected to a gene editing procedure, a cell from a pathogenic organism, a cell obtained from a forensic sample, a cell obtained from an archeological sample, and a cell obtained from a paleontological sample. In one embodiment of any of the above methods, the sample is a cell from a preimplantation embryo (e.g., a blastomere [e.g., a blastomere obtained from an eight-cell stage embryo produced by in vitro fertilization]). In one specific embodiment, the method further comprises determining the presence of disease predisposing germline or somatic variants in the embryo cell. In one embodiment of any of the above methods, the sample is a cell from a pathogenic organism (e.g., a bacterium, a fungus, a protozoan). In one specific embodiment, the pathogenic organism cell is obtained from fluid taken from a patient, microbiota sample (e.g., GI microbiota sample, vaginal microbiota sample, skin microbiota sample, etc.) or an indwelling medical device (e.g., an intravenous catheter, a urethral catheter, a cerebrospinal shunt, a prosthetic valve, an artificial joint, an endotracheal tube, etc.). In one specific embodiment, the method further comprises the step of determining the identity of the pathogenic organism. In one specific embodiment, the method further comprises determining the presence of genetic variants responsible for resistance of the pathogenic organism to a treatment. In one embodiment of any of the above methods, the sample is a tumor cell, a suspected cancer cell, or a cancer cell. In one specific embodiment, the method further comprises determining the presence of one or more diagnostic or prognostic mutations. In one specific embodiment, the method further comprises determining the presence of germline or somatic variants responsible for resistance to a treatment. In one embodiment of any of the above methods, the sample is a cell subjected to a gene editing procedure. In one specific embodiment, the method further comprises determining the presence of unplanned mutations caused by the gene editing process. In one embodiment of any of the above methods, the method further comprises determining the history of a cell lineage. In a related aspect, the invention provides a use of any of the above methods for identifying low frequency sequence variants (e.g., variants which constitute ≥0.01% of the total sequences).

In a related aspect, the invention provides a kit comprising a nucleic acid polymerase, one or more amplification primers, a mixture of nucleotides comprising one or more terminator nucleotides, and optionally instructions for use. In one embodiment of the kits of the invention, the nucleic acid polymerase is a strand displacing DNA polymerase. In one embodiment of the kits of the invention, the nucleic acid polymerase is selected from bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(-) Bst polymerase, exo(-)Bca DNA polymerase, Bsu DNA polymerase, VentR DNA polymerase, VentR (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of the kits of the invention, the nucleic acid polymerase has 3′->5′ exonuclease activity and the terminator nucleotides inhibit such 3′->5′ exonuclease activity (e.g., nucleotides with modification to the alpha group [e.g., alpha-thio dideoxynucleotides], C3 spacer nucleotides, locked nucleic acids (LNA), inverted nucleic acids, 2′ fluoro nucleotides, 3′ phosphorylated nucleotides, 2′-0-Methyl modified nucleotides, trans nucleic acids). In one embodiment of the kits of the invention, the nucleic acid polymerase does not have 3′->5′ exonuclease activity (e.g., Bst DNA polymerase, exo(-) Bst polymerase, exo(-) Bca DNA polymerase, Bsu DNA polymerase, VentR (exo-) DNA polymerase, Deep Vent (exo-) DNA polymerase, Klenow Fragment (exo-) DNA polymerase, Therminator DNA polymerase). In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

Described herein are methods of amplifying a genome, the method comprising: a) bringing into contact a sample comprising the genome, a plurality of amplification primers (e.g., two or more primers), a nucleic acid polymerase, and a mixture of nucleotides which comprises one or more terminator nucleotides which terminate nucleic acid replication by the polymerase, and b) incubating the sample under conditions that promote replication of the genome to obtain a plurality of terminated amplification products, wherein the replication proceeds by strand displacement replication. In one embodiment of any of the above methods, the method further comprises isolating from the plurality of terminated amplification products the products which are between about 50 and about 2000 nucleotides in length. In one embodiment of any of the above methods, the method further comprises isolating from the plurality of terminated amplification products the products which are between about 400 and about 600 nucleotides in length. In one embodiment of any of the above methods, the method further comprises: c) repairing ends and A-tailing, and d) ligating the molecules obtained in step (c) to adaptors, and thereby generating a library of amplification products. In one embodiment of any of the above methods, the method further comprises sequencing the amplification products. In one embodiment of any of the above methods, the amplification is performed under substantially isothermic conditions. In one embodiment of any of the above methods, the nucleic acid polymerase is a DNA polymerase.

In one embodiment of any of the above methods, the DNA polymerase is a strand displacing DNA polymerase. In one embodiment of any of the above methods, the nucleic acid polymerase is selected from bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(-) Bst polymerase, exo(-)Bca DNA polymerase, Bsu DNA polymerase, VentR DNA polymerase, VentR (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of any of the above methods, the nucleic acid polymerase has 3′->5′ exonuclease activity and the terminator nucleotides inhibit such 3′->5′ exonuclease activity. In one specific embodiment, the terminator nucleotides are selected from nucleotides with modification to the alpha group (e.g., alpha-thio dideoxynucleotides creating a phosphorothioate bond), C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. In one embodiment of any of the above methods, the nucleic acid polymerase does not have 3′->5′ exonuclease activity. In one specific embodiment, the polymerase is selected from Bst DNA polymerase, exo(-) Bst polymerase, exo(-) Bca DNA polymerase, Bsu DNA polymerase, VentR (exo-) DNA polymerase, Deep Vent (exo-) DNA polymerase, Klenow Fragment (exo-) DNA polymerase, and Therminator DNA polymerase. In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-0-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In one embodiment of any of the above methods, the amplification primers are between 4 and 70 nucleotides long. In one embodiment of any of the above methods, the amplification products are between about 50 and about 2000 nucleotides in length. In one embodiment of any of the above methods, the target nucleic acid is DNA (e.g., a cDNA or a genomic DNA). In one embodiment of any of the above methods, the amplification primers are random primers. In one embodiment of any of the above methods, the amplification primers comprise a barcode. In one specific embodiment, the barcode comprises a cell barcode. In one specific embodiment, the barcode comprises a sample barcode. In one embodiment of any of the above methods, the amplification primers comprise a unique molecular identifier (UMI). In one embodiment of any of the above methods, the method comprises denaturing the target nucleic acid or genomic DNA before the initial primer annealing. In one specific embodiment, denaturation is conducted under alkaline conditions followed by neutralization. In one embodiment of any of the above methods, the sample, the amplification primers, the nucleic acid polymerase, and the mixture of nucleotides are contained in a microfluidic device. In one embodiment of any of the above methods, the sample, the amplification primers, the nucleic acid polymerase, and the mixture of nucleotides are contained in a droplet. In one embodiment of any of the above methods, the sample is selected from tissue(s) samples, cells, biological fluid samples (e.g., blood, urine, saliva, lymphatic fluid, cerebrospinal fluid (CSF), amniotic fluid, pleural fluid, pericardial fluid, ascites, aqueous humor), bone marrow samples, semen samples, biopsy samples, cancer samples, tumor samples, cell lysate samples, forensic samples, archaeological samples, paleontological samples, infection samples, production samples, whole plants, plant parts, microbiota samples, viral preparations, soil samples, marine samples, freshwater samples, household or industrial samples, and combinations and isolates thereof. In one embodiment of any of the above methods, the sample is a cell (e.g., an animal cell [e.g., a human cell], a plant cell, a fungal cell, a bacterial cell, and a protozoal cell). In one specific embodiment, the cell is lysed prior to the replication. In one specific embodiment, cell lysis is accompanied by proteolysis. In one specific embodiment, the cell is selected from a cell from a preimplantation embryo, a stem cell, a fetal cell, a tumor cell, a suspected cancer cell, a cancer cell, a cell subjected to a gene editing procedure, a cell from a pathogenic organism, a cell obtained from a forensic sample, a cell obtained from an archeological sample, and a cell obtained from a paleontological sample. In one embodiment of any of the above methods, the sample is a cell from a preimplantation embryo (e.g., a blastomere [e.g., a blastomere obtained from an eight-cell stage embryo produced by in vitro fertilization]). In one specific embodiment, the method further comprises determining the presence of disease predisposing germline or somatic variants in the embryo cell. In one embodiment of any of the above methods, the sample is a cell from a pathogenic organism (e.g., a bacterium, a fungus, a protozoan). In one specific embodiment, the pathogenic organism cell is obtained from fluid taken from a patient, microbiota sample (e.g., GI microbiota sample, vaginal microbiota sample, skin microbiota sample, etc.) or an indwelling medical device (e.g., an intravenous catheter, a urethral catheter, a cerebrospinal shunt, a prosthetic valve, an artificial joint, an endotracheal tube, etc.). In one specific embodiment, the method further comprises the step of determining the identity of the pathogenic organism. In one specific embodiment, the method further comprises determining the presence of genetic variants responsible for resistance of the pathogenic organism to a treatment. In one embodiment of any of the above methods, the sample is a tumor cell, a suspected cancer cell, or a cancer cell. In one specific embodiment, the method further comprises determining the presence of one or more diagnostic or prognostic mutations. In one specific embodiment, the method further comprises determining the presence of germline or somatic variants responsible for resistance to a treatment. In one embodiment of any of the above methods, the sample is a cell subjected to a gene editing procedure. In one specific embodiment, the method further comprises determining the presence of unplanned mutations caused by the gene editing process. In one embodiment of any of the above methods, the method further comprises determining the history of a cell lineage. In a related aspect, the invention provides a use of any of the above methods for identifying low frequency sequence variants (e.g., variants which constitute ≥0.01% of the total sequences).

In a related aspect, the invention provides a kit comprising a reverse transcriptase, a nucleic acid polymerase, one or more amplification primers, a mixture of nucleotides comprising one or more terminator nucleotides, and optionally instructions for use. In one embodiment of the kits of the invention, the nucleic acid polymerase is a strand displacing DNA polymerase. In some instances, the reverse transcriptase perform template switching. In some instances, the reverse transcriptase is a variant of MMLV (Moloney Murine Leukemia Virus), HIV-1, AMV (avian myeloblastosis virus), telomerase RT, FIV (feline immunodeficiency virus), or XMRV (Xenotropic murine leukemia virus-related virus. Non-limiting examples of reverse transcriptases include SuperScript I (Thermo), SuperScript II (Thermo), SuperScript III (Thermo), SuperScript IV (Thermo), OmniScript (Qiagen), SensiScript (Qiagen), PrimeScript (Takara), Maxima H-(Thermo), AcuuScript Hi-Fi (Agilent), iScript (Bio-Rad), eAMV (Merck KGaA), qScript (Quanta Biosciences), SmartScribe (Clontech), or GoScript (Promega). In one embodiment of the kits of the invention, the nucleic acid polymerase is selected from bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(-) Bst polymerase, exo(-)Bca DNA polymerase, Bsu DNA polymerase, VentR DNA polymerase, VentR (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of the kits of the invention, the nucleic acid polymerase has 3′->5′ exonuclease activity and the terminator nucleotides inhibit such 3′->5′ exonuclease activity (e.g., nucleotides with modification to the alpha group [e.g., alpha-thio dideoxynucleotides], C3 spacer nucleotides, locked nucleic acids (LNA), inverted nucleic acids, 2′ fluoro nucleotides, 3′ phosphorylated nucleotides, 2′-O-Methyl modified nucleotides, trans nucleic acids). In one embodiment of the kits of the invention, the nucleic acid polymerase does not have 3′->5′ exonuclease activity (e.g., Bst DNA polymerase, exo(-) Bst polymerase, exo(-) Bca DNA polymerase, Bsu DNA polymerase, VentR (exo-) DNA polymerase, Deep Vent (exo-) DNA polymerase, Klenow Fragment (exo-) DNA polymerase, Therminator DNA polymerase). In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-0-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, a kit comprises at least one enzyme stabilizer, neutralization buffer, denaturing buffer, or combination thereof. In some instances, a kit comprises one or more modules. In some instances, a kit comprises a genome module and a transcriptome module.

### Numbered Embodiments

Described herein are the following numbered embodiments 1-46. 1. Described herein are embodiments comprising a method of multiomic single-cell analysis comprising: a. isolating a single cell from a population of cells; b. sequencing a cDNA library comprising polynucleotides amplified from mRNA transcripts from the cell; and c. sequencing the genome of the cell, wherein sequencing the genome of the cell comprises: i. providing a genome from a single cell; ii. contacting the genome with at least one amplification primer, at least one nucleic acid polymerase, and a mixture of nucleotides, wherein the mixture of nucleotides comprises at least one terminator nucleotide which terminates nucleic acid replication by the polymerase, and iii. amplifying at least some of the genome to generate a plurality of terminated amplification products, wherein the replication proceeds by strand displacement replication; iv. ligating the molecules obtained in step (iii) to adaptors, thereby generating a genomic DNA library; and v. sequencing the genomic DNA library. 2. Further provided herein is a method of embodiment 1, wherein the method further comprises identifying at least one protein on the cell surface. 3. Further provided herein is a method of embodiment 1, wherein the mRNA transcripts comprise polyadenylated mRNA transcripts. 4. Further provided herein is a method of embodiment 1, wherein the mRNA transcripts do not comprise polyadenylated mRNA transcripts. 5. Further provided herein is a method of any one of embodiments 1-4, wherein sequencing a cDNA library comprises amplification of mRNA transcripts with template-switching primers. 6. Further provided herein is a method of any one of embodiments 1-4, wherein at least some of the polynucleotides of the cDNA library comprise a barcode. 7. Further provided herein is a method of any one of embodiments 1-4, wherein at least some of the polynucleotides of the cDNA library comprise at least two barcodes. 8. Further provided herein is a method of embodiment 6 or 7, wherein the barcode comprises a cell barcode. 9. Further provided herein is a method of embodiment 6 or 7, wherein the barcode comprises a sample barcode. 10. A method of multiomic single-cell analysis comprising: a. isolating a single cell from a population of cells; b. identifying at least one protein on the cell surface; and c. sequencing the genome of the cell, wherein sequencing the genome of the cell comprises: i. providing a genome from a single cell; ii. contacting the genome with at least one amplification primer, at least one nucleic acid polymerase, and a mixture of nucleotides, wherein the mixture of nucleotides comprises at least one terminator nucleotide which terminates nucleic acid replication by the polymerase; iii. amplifying at least some of the genome to generate a plurality of terminated amplification products, wherein the replication proceeds by strand displacement replication; iv. ligating the molecules obtained in step (iii) to adaptors, thereby generating a genomic DNA library; and v. sequencing the genomic DNA library. 11. Further provided herein is a method of embodiment 10, wherein identifying at least one protein on the cell surface comprises contacting the cell with a labeled antibody which binds to the at least one protein. 12. Further provided herein is a method of embodiment 11, wherein the labeled antibody comprises at least one fluorescent label. 13. Further provided herein is a method of embodiment 11, wherein the labeled antibody comprises at least one mass-tag. 14. Further provided herein is a method of embodiment 11, wherein the labeled antibody comprises at least one nucleic acid barcode. 15. A method of multiomic single-cell analysis comprising: a. isolating a single cell from a population of cells; b. sequencing the genome of the cell, wherein sequencing the genome of the cell comprises: i. providing a genome from a single cell; ii. digesting the genome with a methylation-sensitive restriction enzyme to generate genomic fragments; iii. contacting at least some of the genomic fragments with at least one amplification primer, at least one nucleic acid polymerase, and a mixture of nucleotides, wherein the mixture of nucleotides comprises at least one terminator nucleotide which terminates nucleic acid replication by the polymerase; iv. amplifying at least some of the genome to generate a plurality of terminated amplification products, wherein the replication proceeds by strand displacement replication; v. amplifying at least some of the genomic fragments with methylation-specific PCR; vi. ligating the molecules obtained in steps (iv and v) to adaptors, thereby generating a genomic DNA library and a methylome DNA library; and vii. sequencing the genomic DNA library and the methylome library. 16. Further provided herein is a method of embodiment 15, wherein identifying at least one protein on the cell surface comprises contacting the cell with a labeled antibody which binds to the at least one protein. 17. Further provided herein is a method of embodiment 16, wherein the labeled antibody comprises at least one fluorescent label. 18. Further provided herein is a method of embodiment 16, wherein the labeled antibody comprises at least one mass-tag. 19. Further provided herein is a method of embodiment 16, wherein the labeled antibody comprises at least one nucleic acid barcode. 20. Further provided herein is a method of any one of embodiments 1-19, wherein the single cell is a mammalian cell. 21. Further provided herein is a method of any one of embodiments 1-19, wherein the single cell is a human cell. 22. Further provided herein is a method of any one of embodiments 1-19, wherein the single cells originate from liver, skin, kidney, blood, or lung. 23. Further provided herein is a method of any one of embodiments 1-19, wherein the single cell is a primary cell. 24. Further provided herein is a method of any one of embodiments 1-23, wherein the method further comprises removing at least one terminator nucleotide from the terminated amplification products. 25. Further provided herein is a method of any one of embodiments 1-23, wherein at least some of the amplification products comprise a barcode. 26. Further provided herein is a method of any one of embodiments 1-23, wherein at least some of the amplification products comprise at least two barcodes. 27. Further provided herein is a method of embodiment 24 or 26, wherein the barcode comprises a cell barcode. 28. Further provided herein is a method of embodiment 24 or 26, wherein the barcode comprises a sample barcode. 29. Further provided herein is a method of any one of embodiments 1-28, wherein at least some of the amplification primers comprise a unique molecular identifier (UMI). 30. Further provided herein is a method of any one of embodiments 1-28, wherein at least some of the amplification primers comprise at least two unique molecular identifiers (UMIs). 31. Further provided herein is a method of any one of embodiments 1-30, wherein the method further comprises an additional amplification step using PCR. 32. Further provided herein is a method of any one of embodiments 1-30, wherein at least one mutation is identified in the genome of the cell, wherein the mutation differs from a corresponding position in a reference sequence. 33. Further provided herein is a method of embodiment 32, wherein the at least one mutation occurs in less than 50% of the population of cells. 34. Further provided herein is a method of embodiment 32, wherein the at least one mutation occurs in less than 25% of the population of cells. 35. Further provided herein is a method of embodiment 32, wherein the at least one mutation occurs in less than 1% of the population of cells. 36. Further provided herein is a method of embodiment 32, wherein the at least one mutation occurs in no more than 0.1% of the population of cells. 37. Further provided herein is a method of embodiment 32, wherein the at least one mutation occurs in no more than 0.01% of the population of cells. 38. Further provided herein is a method of embodiment 32, wherein the at least one mutation occurs in no more than 0.001% of the population of cells. 39. Further provided herein is a method of embodiment 32, wherein the at least one mutation occurs in no more than 0.0001% of the population of cells. 40. Further provided herein is a method of embodiment 32, wherein the at least one mutation occurs in no more than 50% of the amplification product sequences. 41. Further provided herein is a method of embodiment 32, wherein the at least one mutation occurs in no more than 25% of the amplification product sequences. 42. Further provided herein is a method of embodiment 32, wherein the at least one mutation occurs in no more than 1% of the amplification product sequences. 43. Further provided herein is a method of embodiment 32, wherein the at least one mutation occurs in no more than 0.1% of the amplification product sequences. 44. Further provided herein is a method of embodiment 32, wherein the at least one mutation occurs in no more than 0.01% of the amplification product sequences. 45. Further provided herein is a method of embodiment 32, wherein the at least one mutation occurs in no more than 0.001% of the amplification product sequences. 46. Further provided herein is a method of embodiment 32, wherein the at least one mutation occurs in no more than 0.0001% of the amplification product sequences.

### EXAMPLES

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Example 1

### Primary Template-Directed Amplification (PTA)

While PTA can be used for any nucleic acid amplification, it is particularly useful for whole genome amplification as it allows to capture a larger percentage of a cell genome in a more uniform and reproducible manner and with lower error rates than the currently used methods such as, e.g., Multiple Displacement Amplification (MDA), avoiding such drawbacks of the currently used methods as exponential amplification at locations where the polymerase first extends the random primers which results in random overrepresentation of loci and alleles and mutation propagation (see FIG. 1G). PTA is also used with other analysis techniques, such as transcriptome analysis.

Cell Culture

Human NA12878 (Coriell Institute) cells were maintained in RPMI media, supplemented with 15% FBS and 2 mM L-glutamine, and 100 units/mL of penicillin, 100 μg/mL of streptomycin, and 0.25 μg/mL of Amphotericin B (Gibco, Life Technologies). The cells were seeded at a density of 3.5×10⁵ cells/ml. The cultures were split every 3 days and were maintained in a humidified incubator at 37C with 5% CO₂.

Single-Cell Isolation and WTA

A general protocol for WTA (whole transcriptome analysis) is shown in FIG. 2F. Cells were resuspended at a concentration of 150-500 cells/μL. This cell suspension was stained with 20 μL of freshly prepared staining buffer (2.5 μL ethidium homodimer-1 and 0.625 μL Calcein AM from Life Technology's LIVE/DEAD® Viability/Cytotoxicity Kit added to 1.25 mL cell buffer containing 1×PBS and 0.05% tween-20. Cells were then sorted using a FACS Aria III sorting instrument to deposit cells in each of the 96 wells. A reaction mix containing 5× RT Buffer, PEG4000, RT primer (100 uM), TS Oligo (20 uM), reverse transcriptase, RNAse inhibitor, Gelatin, Tween-20, Triton-×, dNTP mix, TMAC (1M), Betaine (5M), MgCl₂ (50mM), ERCC spikes were added to each well. The sample was then placed on a thermal cycler for 90 min at 42C, 30 min at 50C, and then held at 4C until the sample could be processed for Pre-Amplification. After thermal cycling for RT, the sample is either processed for DNA amplification or pre-amplification of first strand cDNA resulting from the RT reaction. Preamplification of the sample was accomplished using a single primer (semi-suppressive PCR) with the following protocol to amplify the cDNA products. Briefly a 5 uL of the RT reaction was added to a 30 microliter reaction containing 2× master mix, 1 micromolar primer and 5× Preamp buffer using the following thermal cycling conditions 95C-1 min. 21 cycles of 95C-15 sec, 60C-30 sec, 68C-4 min, followed by a hold at 72C for a period of ten minutes. Samples were then converted into a sequencing library using the Nextera XT library prep kit using the manufacturer's instruction (FIG. 2G). Results for the RT experiment are shown for six samples in Table 1.

TABLE 1

| Sample | Map to | Map to | Gene’s | Reads/ | |
|---|---|---|---|---|---|
| # | Genome | transcriptome | detected | sample | Cells |
| 1 | 99.46% | 94.24% | 9298 | 2,530,484 | ~350 |
| 2 | 99.48% | 94.17% | 9692 | 2,239,730 | ~350 |
| 3 | 99.45% | 94.77% | 3630 | 2,466,899 | ~35 |
| 4 | 98.91% | 94.45% | 4351 | 2,235,999 | ~35 |
| 5 | 98.72% | 92.70% | 2189 | 1,788,750 | ~3 |
| 6 | 96.13% | 89.92% | 4272 | 1,565,289 | ~3 |

Single-Cell Isolation and WGA

After culturing NA12878 cells for a minimum of three days after seeding at a density of 3.5×10⁵ cells/ml, 3 mL of cell suspension were pelleted at 300×g for 10 minutes. The medium was then discarded and the cells were washed three times with 1mL of cell wash buffer (1×PBS containing 2% FBS without Mg² or Ca²) being spun at 300×g, 200×g and finally 100×g for 5 minutes. The cells were then resuspended in 500 μL of cell wash buffer. This was followed by staining with 100 nM of Calcein AM (Molecular Probes) and 100 ng/ml of propidium iodide (PI; Sigma-Aldrich) to distinguish the live cell population. The cells were loaded on a BD FACScan flow cytometer (FACSAria II) (BD Biosciences) that had been thoroughly cleaned with ELIMINase (Decon Labs) and calibrated using Accudrop fluorescent beads (BD Biosciences) for cell sorting. A single cell from the Calcein AM-positive, PI-negative fraction was sorted in each well of a 96 well plate containing 3 μL of PBS with 0.2% Tween 20 in the cells that would undergo PTA (Sigma-Aldrich). Multiple wells were intentionally left empty to be used as no template controls (NTC). Immediately after sorting, the plates were briefly centrifuged and placed on ice. Cells were then frozen at a minimum of overnight at −20° C. On a subsequent day, WGA Reactions were assembled on a pre-PCR workstation that provides a constant positive pressure of HEPA filtered air and which was decontaminated with UV light for 30 minutes before each experiment.

MDA was carried out with modifications that have previously been shown to improve the amplification uniformity. Specifically, exonuclease-resistant random primers (ThermoFisher) were added to a lysis buffer/mix to a final concentration of 125 μM. 4 μL of the resulting lysis/denaturing mix was added to the tubes containing the single cells, vortexed, briefly spun and incubated on ice for 10 minutes. The cell lysates were neutralized by adding 3 μL of a quenching buffer, mixed by vortexing, centrifuged briefly, and placed at room temperature. This was followed by addition of 40 μl of amplification mix before incubation at 30° C. for 8 hours after which the amplification was terminated by heating to 65° C. for 3 minutes.

PTA was carried out by first further lysing the cells after freeze thawing by adding 2 μl a prechilled solution of a 1:1 mixture of 5% Triton X-100 (Sigma-Aldrich) and 20 mg/ml Proteinase K (Promega). The cells were then vortexed and briefly centrifuged before placing at 40 degrees for 10 minutes. 4 μl of lysis buffer/mix and 1 μl of 500 μM exonuclease-resistant random primer were then added to the lysed cells to denature the DNA prior to vortexing, spinning, and placing at 65 degrees for 15 minutes. 4 μl of room temperature quenching buffer was then added and the samples were vortexed and spun down. 56 μl of amplification mix (primers, dNTPs, polymerase, buffer) that contained alpha-thio-ddNTPs at equal ratios at a concentration of 1200 μM in the final amplification reaction. The samples were then placed at 30° C. for 8 hours after which the amplification was terminated by heating to 65° C. for 3 minutes.

After the amplification step, the DNA from both MDA and PTA reactions were purified using AMPure XP magnetic beads (Beckman Coulter) at a 2:1 ratio of beads to sample and the yield was measured using the Qubit dsDNA HS Assay Kit with a Qubit 3.0 fluorometer according to the manufacturer's instructions (Life Technologies).

Library Preparation

The MDA reactions resulted in the production of 40 μg of amplified DNA. 1 μg of product was fragmented for 30 minutes according to standard procedures. The samples then underwent standard library preparation with 15 μM of dual index adapters (end repair by a T4 polymerase, T4 polynucleotide kinase, and Taq polymerase for A-tailing) and 4 cycles of PCR. Each PTA reaction generated between 40-60 ng of material which was used for standard DNA sequencing library preparation in its entirety without fragmentation. 2.5 μM adapters with UMIs and dual indices were used in the ligation with T4 ligase, and 15 cycles of PCR (hot start polymerase) were used in the final amplification. The libraries were then cleaned up using a double sided SPRI using ratios of 0.65× and 0.55× for the right and left sided selection, respectively. The final libraries were quantified using the Qubit dsDNA BR Assay Kit and 2100 Bioanalyzer (Agilent Technologies) before sequencing on the Illumina NextSeq platform. All Illumina sequencing platforms, including the NovaSeq, are also compatible with the protocol.

Data Analysis

Sequencing reads were demultiplexed based on cell barcode using Bcl2fastq. The reads were then trimmed using trimmomatic, which was followed by alignment to hg19 using BWA. Reads underwent duplicate marking by Picard, followed by local realignment and base recalibration using GATK 4.0. All files used to calculate quality metrics were downsampled to twenty million reads using Picard DownSampleSam. Quality metrics were acquired from the final bam file using qualimap, as well as Picard AlignmentSummaryMetrics and CollectWgsMetrics. Total genome coverage was also estimated using Preseq.

Variant Calling

Single nucleotide variants and Indels were called using the GATK UnifiedGenotyper from GATK 4.0. Standard filtering criteria using the GATK best practices were used for all steps in the process (https://software.broadinstitute.org/gatk/best-practices/). Copy number variants were called using Control-FREEC (Boeva et al., Bioinformatics, 2012, 28(3):423-5). Structural variants were also detected using CREST (Wang et al., Nat Methods, 2011, 8(8):652-4).

Results

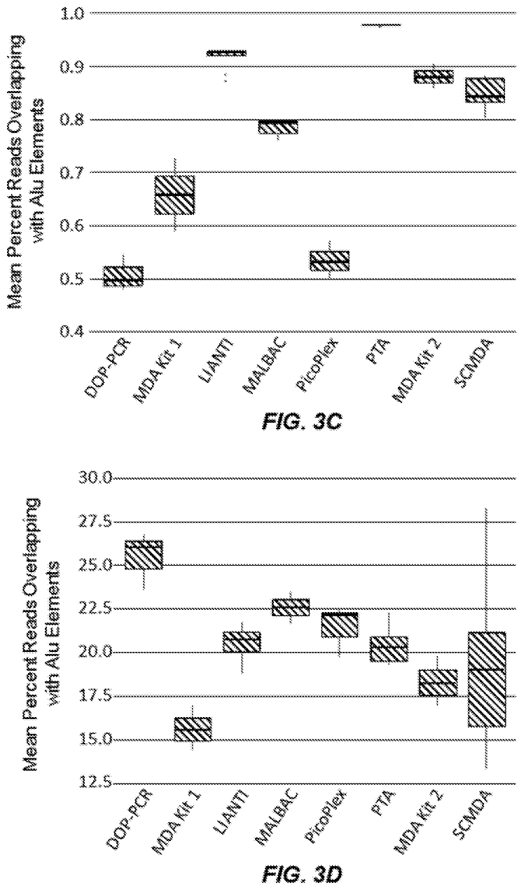

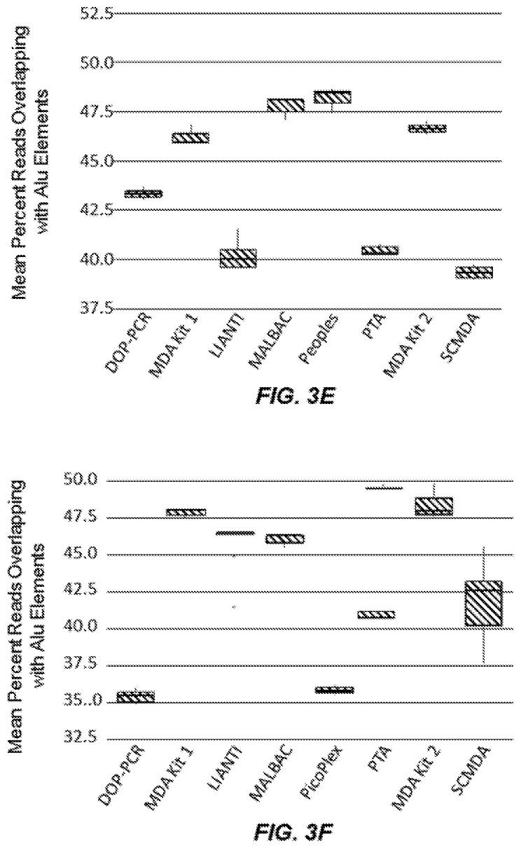

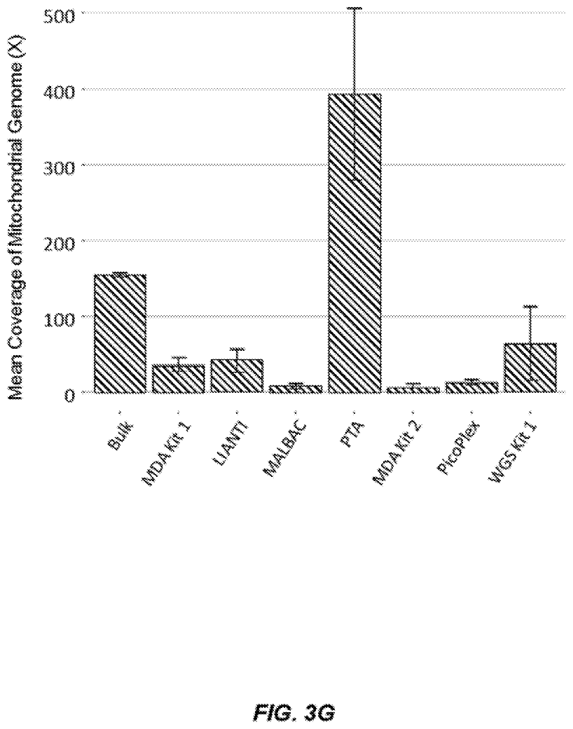

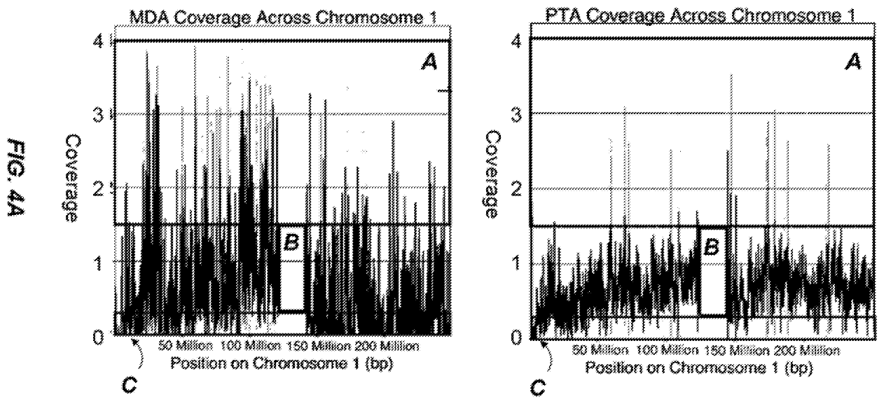

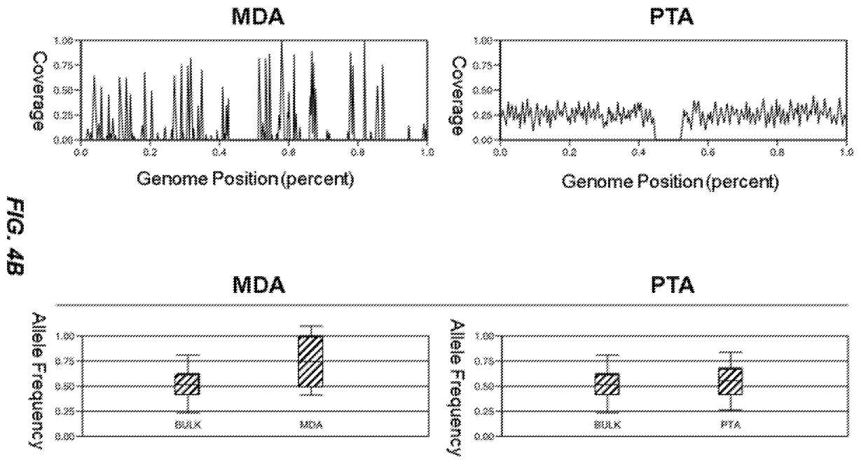

As shown in FIG. 3A and FIG. 3B, the mapping rates and mapping quality scores of the amplification with dideoxynucleotides (“reversible”) alone are 15.0 +/− 2.2 and 0.8 +/− 0.08, respectively, while the incorporation of exonuclease-resistant alpha-thio dideoxynucleotide terminators (“irreversible”) results in mapping rates and quality scores of 97.9 +/− 0.62 and 46.3 +/× 3.18, respectively. Experiments were also run using a reversible ddNTP, and different concentrations of terminators. (FIG. 2A, bottom)

FIGS. 2B-2E show the comparative data produced from NA12878 human single cells that underwent MDA (following the method of Dong, ×. et al., Nat Methods. 2017, 14(5):491-493) or PTA. While both protocols produced comparable low PCR duplication rates (MDA 1.26% +/− 0.52 vs PTA 1.84% +/− 0.99). and GC% (MDA 42.0 +/− 1.47 vs PTA 40.33 +/− 0.45), PTA produced smaller amplicon sizes. The percent of reads that mapped and mapping quality scores were also significantly higher for PTA as compared to MDA (PTA 97.9 +/− 0.62 vs MDA 82.13 +/− 0.62 and PTA 46.3 +/− 3.18 vs MDA 43.2 +/− 4.21, respectively). Overall, PTA produces more usable, mapped data when compared to MDA. FIG. 4A shows that, as compared to MDA, PTA has significantly improved uniformity of amplification with greater coverage breadth and fewer regions where coverage falls to near 0. The use of PTA allows identifying low frequency sequence variants in a population of nucleic acids, including variants which constitute >0.01% of the total sequences. PTA can be successfully used for single cell genome amplification.

### Example2

### Comparative Analysis of PTA

Benchmarking PTA and SCMDA Cell Maintenance and Isolation

Lymphoblastoid cells from 1000 Genome Project subject NA12878 (Coriell Institute, Camden, N.J., USA) were maintained in RPMI media, which was supplemented with 15% FBS, 2 mM L-glutamine, 100 units/mL of penicillin, 100 μg/mL of streptomycin, and 0.25 μg/mL of Amphotericin B). The cells were seeded at a density of 3.5×10⁵ cells/ml and split every 3 days. They were maintained in a humidified incubator at 37° C. with 5% CO₂. Prior to single cell isolation, 3 mL of suspension of cells that had expanded over the previous 3 days was spun at 300×g for 10 minutes. The pelleted cells were washed three times with 1mL of cell wash buffer (1×PBS containing 2% FBS without Mg²⁺ or Ca²⁺)) where they were spun sequentially at 300×g, 200×g, and finally 100×g for 5 minutes to remove dead cells. The cells were then resuspended in 500 uL of cell wash buffer, which was followed by staining with 100 nM of Calcein AM and 100 ng/ml of propidium iodide (PI) to distinguish the live cell population. The cells were loaded on a BD FACScan flow cytometer (FACSAria II) that had been thoroughly cleaned with ELIMINase and calibrated using Accudrop fluorescent beads. A single cell from the Calcein AM-positive, PI-negative fraction was sorted in each well of a 96 well plate containing 3 uL of PBS with 0.2% Tween 20. Multiple wells were intentionally left empty to be used as no template controls. Immediately after sorting, the plates were briefly centrifuged and placed on ice. Cells were then frozen at a minimum of overnight at −80° C.

PTA and SCMDA Experiments

WGA Reactions were assembled on a pre-PCR workstation that provides constant positive pressure with HEPA filtered air and which was decontaminated with UV light for 30 minutes before each experiment. MDA was carried according to the SCMDA methodology using the published protocol (Dong et al. Nat. Meth. 2017, 14, 491-493). Specifically, exonuclease-resistant random primers were added at a final concentration of 12.5 uM to the lysis buffer. 4 uL of the resulting lysis mix was added to the tubes containing the single cells, pipetted three times to mix, briefly spun and incubated on ice for 10 minutes. The cell lysates were neutralized by adding 3 uL of quenching buffer, mixed by pipetting 3 times, centrifuged briefly, and placed on ice. This was followed by addition of 40 ul of amplification mix before incubation at 30° C. for 8 hours after which the amplification was terminated by heating to 65° C. for 3 minutes. PTA was carried out by first further lysing the cells after freeze thawing by adding 2 μl of a prechilled solution of a 1:1 mixture of 5% Triton X-100 and 20 mg/ml Proteinase K. The cells were then vortexed and briefly centrifuged before placing at 40 degrees for 10 minutes. 4 μl of denaturing buffer and 1 μl of 500 μM exonuclease-resistant random primer were then added to the lysed cells to denature the DNA prior to vortexing, spinning, and placing at 65° C. for 15 minutes. 4 μl of room temperature quenching solution was then added and the samples were vortexed and spun down. 56 μl of amplification mix that contained alpha-thio-ddNTPs at equal ratios at a concentration of 1200 μM in the final amplification reaction. The samples were then placed at 30° C. for 8 hours after which the amplification was terminated by heating to 65° C. for 3 minutes. After the SCMDA or PTA amplification, the DNA was purified using AMPure XP magnetic beads at a 2:1 ratio of beads to sample and the yield was measured using the Qubit dsDNA HS Assay Kit with a Qubit 3.0 fluorometer according to the manufacturer's instructions.

Library Preparation

1 ug of SCMDA product was fragmented for 30 minutes according to the HyperPlus protocol after the addition of the conditioning solution. The samples then underwent standard library preparation with 15 uM of unique dual index adapters and 4 cycles of PCR. The entire product of each PTA reaction was used for DNA sequencing library preparation using standard amplification protocols without fragmentation. 2.5 uM of unique dual index adapter was used in the ligation, and 15 cycles of PCR were used in the final amplification. The libraries from SCMDA and PTA were then visualized on a 1% Agarose E-Gel. Fragments between 400-700 bp were excised from the gel and recovered using a Gel DNA Recovery Kit. The final libraries were quantified using the Qubit dsDNA BR Assay Kit and Agilent 2100 Bioanalyzer before sequencing on the NovaSeq 6000.

Data Analysis

Data was trimmed using trimmomatic, which was followed by alignment to hg19 using BWA. Reads underwent duplicate marking by Picard, followed by local realignment and base recalibration using GATK 3.5 best practices. All files were downsampled to the specified number of reads using Picard DownSampleSam. Quality metrics were acquired from the final bam file using qualimap, as well as Picard AlignmentMetricsAummary and CollectWgsMetrics. Lorenz curves were drawn and Gini Indices calculated using htSeqTools. SNV calling was performed using UnifiedGenotyper, which were then filtered using the standard recommended criteria (QD<2.0∥ FS>60.0∥ MQ<40.0∥ SOR>4.0∥ MQRankSum <−12.5∥ ReadPosRankSum <−8.0). No regions were excluded from the analyses and no other data normalization or manipulations were performed. Sequencing metrics for the methods tested are found in Table 2.

TABLE 2
Comparison of sequencing metrics between methods tested.

| | | MDA | | | | MDA | DOP |
|---|---|---|---|---|---|---|---|
| | PTA | Kit 2 | PicoPlex | MALBAC | LIANTI | Kit 1 | PCR |
| % | 97 | 88 | 55 | 79 | 92 | 65 | 52 |
| Genome | | | | | | | |
| Mapping | | | | | | | |
| % | 95 | 75 | 43 | 60 | 82 | 73 | 23 |
| Genome | | | | | | | |
| Recovery | | | | | | | |
| (300M | | | | | | | |
| reads) | | | | | | | |
| CV of | 0.8 | 1.8 | 3 | 2.5 | 1.1 | 2 | 3.5 |
| Coverage | | | | | | | |
| (300M | | | | | | | |
| reads) | | | | | | | |
| SNV | 76 | 50 | 15 | 34 | 49 | 46 | 5 |
| Sensitivity | | | | | | | |
| % (300M | | | | | | | |
| reads) | | | | | | | |
| SNV | 93 | 91 | 56 | 47 | 88 | 90 | 35 |
| Specificity | | | | | | | |
| % (300M | | | | | | | |
| reads) | | | | | | | |

CV = Coefficient of Variation; SNV = Single Nucleotide Variation; values refer to 15× coverage.

Genome Coverage Breadth and Uniformity

Comprehensive comparisons of PTA to all common single-cell WGA methods were performed. To accomplish this, PTA and an improved version of MDA called single-cell MDA (Dong et al. Nat. Meth. 2017, 14, 491-493) (SCMDA) was performed on 10 NA12878 cells each. In addition, those results to cells that had undergone amplification with DOP-PCR (Zhang et al. PNAS 1992, 89, 5847-5851), MDA Kit 1 (Dean et al. PNAS 2002, 99, 5261-5266), MDA Kit 2, MALBAC (Zong et al. Science 2012, 338, 1622-1626), LIANTI(Chen et al., Science 2017, 356, 189-194), or PicoPlex(Langmore, Pharmacogenomics 3, 557-560 (2002)) was compared using data produced as part of the LIANTI study.

To normalize across samples, raw data from all samples were aligned and underwent pre-processing for variant calling using the same pipeline. The bam files were then subsampled to 300 million reads each prior to performing comparisons. Importantly, the PTA and SCMDA products were not screened prior to performing further analyses while all other methods underwent screening for genome coverage and uniformity before selecting the highest quality cells that were used in subsequent analyses. Of note, SCMDA and PTA were compared to bulk diploid NA12878 samples while all other methods were compared to bulk BJ1 diploid fibroblasts that had been used in the LIANTI study. As seen in FIGS. 3C-3F, PTA had the highest percent of reads aligned to the genome, as well as the highest mapping quality. PTA, LIANTI, and SCMDA had similar GC content, all of which were lower than the other methods. PCR duplication rates were similar across all methods. Additionally, the PTA method enabled smaller templates such as the mitochondrial genome to give higher coverage rates (similar to larger canonical chromosomes) relative to other methods tested (FIG. 3G).

Coverage breadth and uniformity of all methods was then compared. Examples of coverage plots across chromosome 1 are shown for SCMDA and PTA, where PTA is shown to have significantly improved uniformity of coverage and allele frequency (FIGS. 4B). Coverage rates were then calculated for all methods using increasing number of reads. PTA approaches the two bulk samples at every depth, which is a significant improvement over all other methods (FIG. 5A). We then used two strategies to measure coverage uniformity. The first approach was to calculate the coefficient of variation of coverage at increasing sequencing depth where PTA was found to be more uniform than all other methods (FIG. 5B). The second strategy was to compute Lorenz curves for each subsampled bam file where PTA was again found to have the greatest uniformity (FIG. 5C). To measure the reproducibility of amplification uniformity, Gini Indices were calculated to estimate the difference of each amplification reaction from perfect uniformity (de Bourcy et al., PloS one 9, e105585 (2014)). PTA was again shown to be reproducibly more uniform than the other methods (FIG. 5D).

SNV Sensitivity

To determine the effects of these differences in the performance of the amplification methods on SNV calling, variant call rates for each to the corresponding bulk sample were compared at increasing sequencing depth. To estimate sensitivity, the percent of variants called in corresponding bulk samples that had been subsampled to 650 million reads that were found in each cell at each sequencing depth (FIG. 5E) were compared. Improved coverage and uniformity of PTA resulted in the detection of 45.6% more variants over MDA Kit 2, which was the next most sensitive method. An examination of sites called as heterozygous in the bulk sample showed that PTA had significantly diminished allelic skewing at those heterozygous sites (FIG. 5F). This finding supports the assertion that PTA not only has more even amplification across the genome, but also more evenly amplifies two alleles in the same cell.

SNV Specificity

To estimate the specificity of mutation calls, the variants called in each single cell not found in the corresponding bulk sample were considered false positives. The lower temperature lysis of SCMDA significantly reduced the number of false positive variant calls (FIG. 5G). Methods using thermostable polymerases (MALBAC, PicoPlex, and DOP-PCR) showed further decreases in the SNV calling specificity with increasing sequencing depth. Without being bound by theory, this is likely the result of the significantly increased error rate of those polymerases compared to phi29 DNA polymerase. In addition, the base change patterns seen in the false positive calls also appear to be polymerase-dependent (FIG. 5H). As seen in FIG. 5G, the model of suppressed error propagation in PTA is supported by the lower false positive SNV calling rate in PTA compared to standard MDA protocols. In addition, PTA has the lowest allele frequencies of false positive variant calls, which is again consistent with the model of suppressed error propagation with PTA (FIG. 5I).

### Example 3

### Massively Parallel Single-Cell DNA Sequencing

Using PTA, a protocol for massively parallel DNA sequencing is established. First, a cell barcode is added to the random primer. Two strategies to minimize any bias in the amplification introduced by the cell barcode is employed: 1) lengthening the size of the random primer and/or 2) creating a primer that loops back on itself to prevent the cell barcode from binding the template (FIG. 10B). Once the optimal primer strategy is established, up to 384 sorted cells are scaled by using, e.g., Mosquito HTS liquid handler, which can pipette even viscous liquids down to a volume of 25 nL with high accuracy. This liquid handler also reduces reagent costs approximately 50-fold by using a 1 μL PTA reaction instead of the standard 50 μL reaction volume.

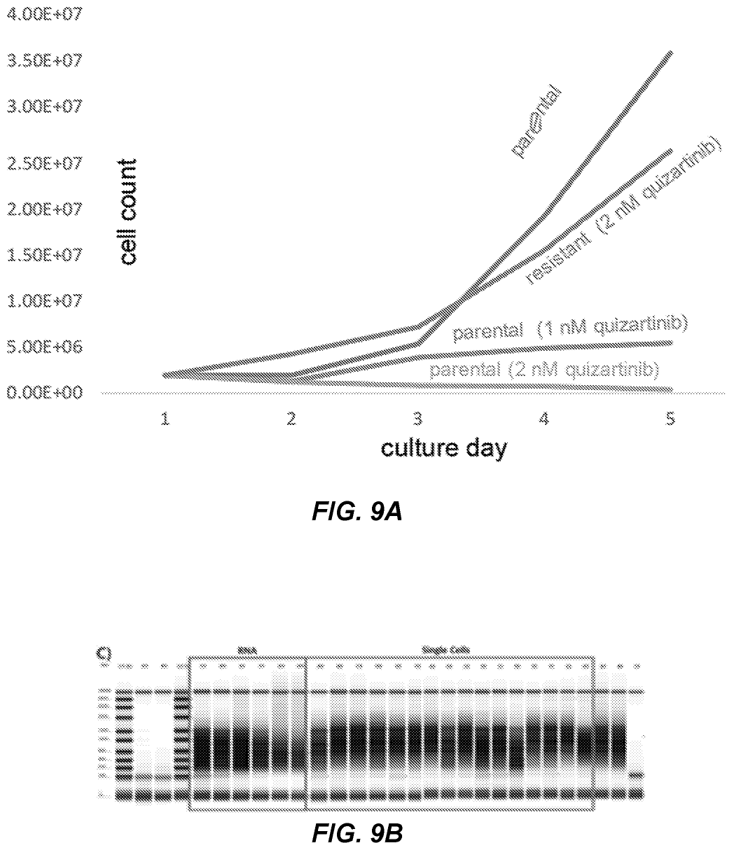

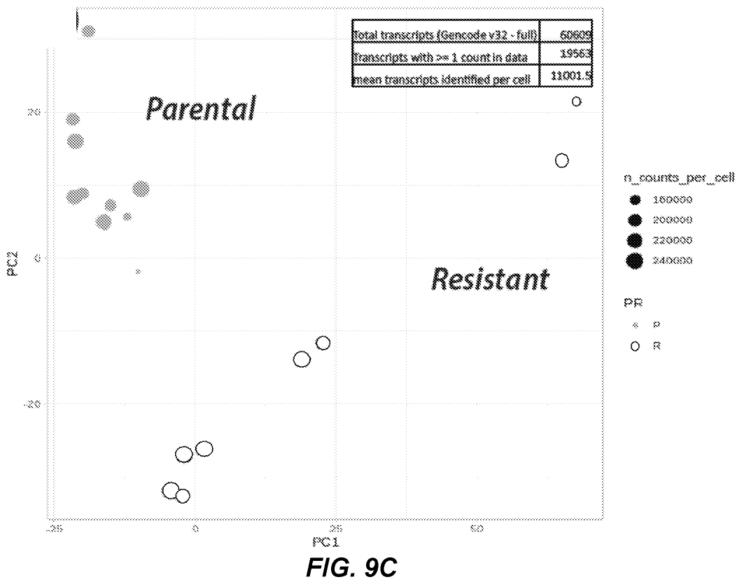

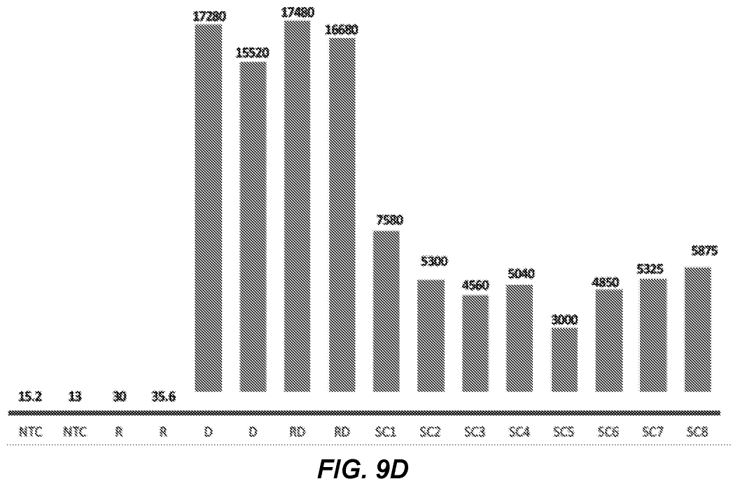

The amplification protocol is transitioned into droplets by delivering a primer with a cell barcode to a droplet. Solid supports, such as beads that have been created using the split-and-pool strategy, are optionally used. Suitable beads are available e.g., from ChemGenes. The oligonucleotide in some instances contains a random primer, cell barcode, unique molecular identifier, and cleavable sequence or spacer to release the oligonucleotide after the bead and cell are encapsulated in the same droplet. During this process, the template, primer, dNTP, alpha-thio-ddNTP, and polymerase concentrations for the low nanoliter volume in the droplets are optimized. Optimization in some instances includes use of larger droplets to increase the reaction volume. As seen in FIG. 9, this process requires two sequential reactions to lyse the cells, followed by WGA. The first droplet, which contains the lysed cell and bead, is combined with a second droplet with the amplification mix. Alternatively or in combination, the cell is encapsulated in a hydrogel bead before lysis and then both beads may be added to an oil droplet. See Lan, F. et al., Nature Biotechnol., 2017, 35:640-646).

Additional methods include use of microwells, which in some instances capture 140,000 single cells in 20-picoliter reaction chambers on a device that is the size of a 3″×2″ microscope slide. Similarly to the droplet-based methods, these wells combine a cell with a bead that contains a cell barcode, allowing massively parallel processing. See Gole et al., Nature Biotechnol., 2013, 31:1126-1132).

### Example 4

### Parallel Analysis of Genomic and Transcriptome in Single Cells

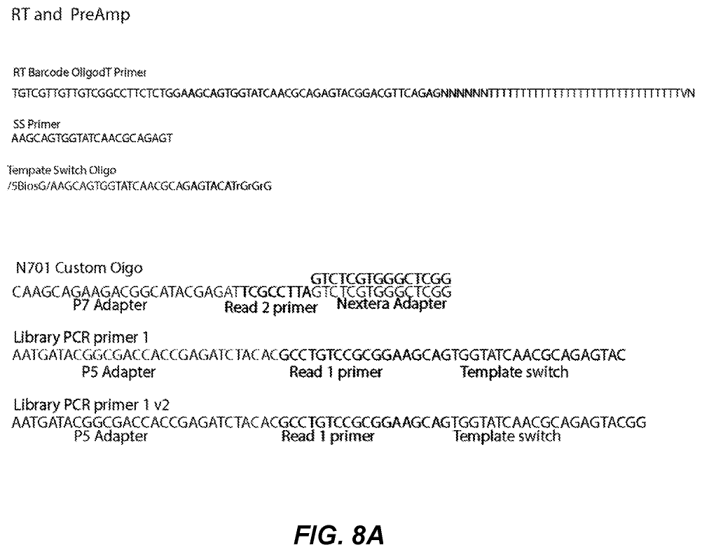

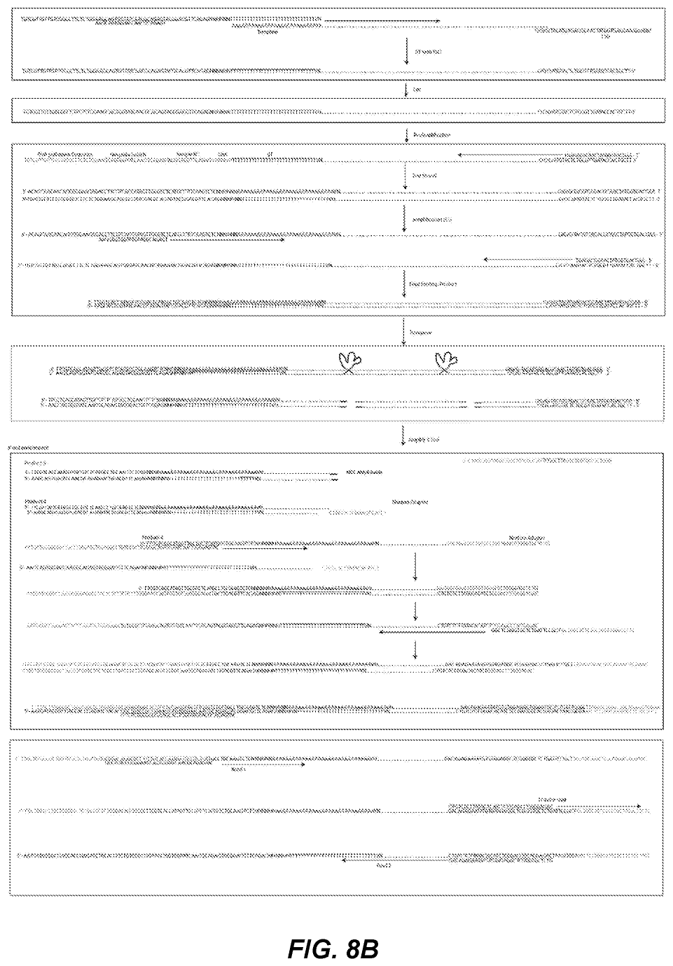

Single cells from a population of cells are sorted, placing one cell per well. Each well comprises an antibody fixed to a region of the surface, wherein the antibody binds to a cell nucleus. The outer membrane of the cell is lysed, releasing mRNA into a solution in the well while the nuclease remains intact and bound to a region of the well. RT is performed using the mRNA in solution as a template to generate cDNA using the primers in FIG. 8A. Optionally, an rRNA (ribosomal RNA) depletion step is conducted. A first template switching primer comprising from 5′ to 3′ a TSS region (transcription start site), an anchor region, a RNA BC region, and a poly dT tail; and a second template switching primer comprising from 3′ to 5′ a TSS region, an anchor region, and a poly G region are used for RT PCR. After removal of RT PCR products (cDNA library) for subsequent sequencing, any remaining RNA in the cell is removed by UNG. The RNA library is prepared using the Nextera/transposon-based sequencing method and reagents (FIG. 8B). The cDNA library comprises short cDNAs with approximately 1000 fold amplification. The nucleus is then lysed, and the released genomic DNA is subjected to the PTA method using random primers with an isothermal polymerase with random primers 6-9 bases in length. Amplification conditions for PTA are selected to generate amplicons of 250-1500 bases in length. PTA products are optionally subjected to additional amplification and sequenced. RNA sequencing data, and DNA sequencing data are compiled into a database for analysis.

### Example 5

### Single Cell Multiomic Analysis

A population of cells is contacted with an antibody library, wherein antibodies are labeled. Antibodies are labeled with either fluorescent labels, nucleic acid barcodes, or both. Labeled antibodies bind to at least one cell in the population, and such cells are sorted, placing one cell per well. Some labeled antibodies provide specific information about cell surface protein markers after binding, which is obtained by either fluorescence microscopy or read of barcodes tagged to the antibodies. Each well comprises an antibody fixed to a region of the surface, wherein the antibody binds to a cell nucleus. The outer membrane of the cell is lysed, releasing mRNA into a solution in the well while the nuclease remains intact and bound to a region of the well. Optionally, an rRNA (ribosomal RNA) depletion step is conducted. Next, RT is performed using the mRNA in solution as a template to generate cDNA. A first template switching primer comprising from 5′ to 3′ a TSS region (transcription start site), an anchor region, a RNA BC region, and a poly dT tail; and a second template switching primer comprising from 3′ to 5′ a TSS region, an anchor region, and a poly G region are used for RT PCR. After removal of RT PCR products (cDNA library) for subsequent sequencing, any remaining RNA in the cell is removed by UNG. The cDNA library comprises short cDNAs with approximately 1000 fold amplification. The nucleus is then lysed, and the released genomic DNA is subjected to the PTA method using random primers with an isothermal polymerase with random primers 6-9 bases in length. Amplification conditions for PTA are selected to generate amplicons of 250-1500 bases in length. PTA products are optionally subjected to additional amplification and sequenced. Protein data, RNA sequencing data, and DNA sequencing data are compiled into a database for analysis.

### Example 6

### Single Cell Analysis of Methylome and Transcriptome

Single cells from a population of cells are sorted, placing one cell per well. Each well comprises an antibody fixed to a region of the surface, wherein the antibody binds to a cell nucleus. The outer membrane of the cell is lysed, releasing mRNA into a solution in the well while the nuclease remains intact and bound to a region of the well. mRNA transcripts are contacted with a terminal transferase to add riboguanine to the 5′ end of the mRNA strands. Next, RT is performed using the mRNA in solution as a template to generate cDNA. Optionally, an rRNA (ribosomal RNA) depletion step is conducted. A first template switching primer comprising from 5′ to 3′ a TSS region (transcription start site), an anchor region, a RNA BC region, and a poly dT tail; and a second template switching primer comprising from 3′ to 5′ a TSS region, an anchor region, and a poly G region are used for RT PCR. After removal of RT PCR products (cDNA library) for subsequent sequencing, any remaining RNA in the cell is removed by UNG. The cDNA library comprises short cDNAs with approximately 1000 fold amplification. The nucleus is then lysed, and the released genomic DNA fragmented using methylation-sensitive endonucleases. The genome fragments are subjected to the PTA method using random primers with an isothermal polymerase with random primers 6-9 bases in length. Amplification conditions for PTA are selected to generate amplicons of 250-1500 bases in length. PTA products are optionally subjected to additional amplification and sequenced. RNA sequencing data, and DNA sequencing data are compiled into a database for analysis, and methylation-sensitive endonuclease cut sites are identified. These sites are used to map locations of methylations on the original genomic DNA.

### Example 7

### Single Cell Analysis of Methylome and Genome

Single cells from a population of cells are sorted, placing one cell per well. Each well comprises an antibody fixed to a region of the surface, wherein the antibody binds to a cell nucleus. Cells are lysed with methylation-sensitive enzymes, and the genomes are subjected to the PTA method using random primers with an isothermal polymerase with random primers 6-9 bases in length. Amplification conditions for PTA are selected to generate amplicons of 250-1500 bases in length. The reaction mixture is split, wherein half the mixture is subjected to exome enrichment, whole genome sequencing, or other targeted sequencing method. The other half of the reaction mixture is subjected to methylation sensitive PCR conditions. Methylation and DNA sequencing data are compiled into a database for analysis.

### Example 8

### Single Cell Analysis of Surface Proteome and Genome

Cells from a sample comprising a population of cells is contacted with a library of baits, such as antibodies, polynucleotides, or other small molecules. In some instances, baits are barcoded (such as barcoded antibodies) to allow pulldown and identification of binding of baits to proteins on cell surfaces. Alternatively or in combination, baits are labeled with other labels, such as fluorescent labels or mass tags. Single cells from the population of cells are sorted, placing one cell per well. Optionally, baits that have bound to the cell surface are removed for sequencing or identification prior to genomic library preparation. Cells are lysed, the genome is released into solution and fragments are generated. The genome fragments are subjected to the PTA method using random primers with an isothermal polymerase with random primers 6-9 bases in length. Alternatively, the genome is not fragmented prior to amplification with PTA. Amplification conditions for PTA are selected to generate amplicons of 250-1500 bases in length. PTA products are optionally subjected to additional amplification and sequenced. Cell-surface proteins and DNA sequencing data are compiled into a database for analysis.

### Example 9

### Multiomics for Measuring Drug Resistance

Monotherapy with small molecule inhibitors targeting FLT3 in AML (Acute Myeloid Leukemia) have shown clinical benefit, but resistance invariably occurs. The FLT3 inhibitor quizartinib (AC220) is one such inhibitor, whereby the drug has yielded a composite complete remission of approximately 50% in relapsed or refractory AML patients. Despite this success, secondary FLT3 mutations in the activation loop (D835) and at the gatekeeper residue F691 have been identified in FLT3-ITD patients who relapsed on quizartinib therapy. Clinical resistance to the multi-kinase inhibitor PKC412 was determined to be the result of a secondary mutation in the FLT3 kinase domain. Additional, FLT3-independent modes of resistance to targeted therapies have been identified in FLT3-ITD AML, including bypass pathway activation of AXL, as well as NRAS, TET2, and IDH1/2 mutations. Mutations in epigenetic modifying enzymes and transcription factors have also been observed, highlighting the complexity and diversity of mechanisms of resistance to FLT3 inhibition.

Quizartinib-resistant and matched parental MOLM-13 AML cell lines, and a cell line harboring a heterozygous FLT3-ITD mutation was generated. The PTA method was combined RNAseq chemistry and used to genomically and transcriptionally probe these drug-resistant single cells in order to gain insight into mechanisms of resistance following FLT3 inhibition in AML. Briefly, the workflow comprised (1) creation of resistant cells, (2) isolation of resistant cells, (3) cytosolic lysis to release mRNA, (4) reverse transcription to generate cDNA from the mRNA, (5) nuclear lysis to release genomic DNA, (6) PTA amplification, (7) separate DNA/RNA enrichment, (8) cDNA PreAMP of enriched mRNA, (9) library preparation, QC, and pooling, (10) next generation sequencing, and (11) data analysis.

Cell Culture. MOLM-13 acute myeloid leukemia cells harboring heterozygous FLT3 internal tandem duplication (ITD)1 were obtained from the DSMZ-German Collection of Microorganisms and Cell Cultures (ACC 554). Cells were maintained in RPMI 1640 (Gibco 11875-093) supplemented with 10% FBS and penicillin/streptomycin, and subcultured every 2-3 days while maintaining a density range of 2.5 E5-0 1.5 E6 cells/ml. For generation of the quizartinib-resistant MOLM-13 line, cells were continually treated with 2 nM quizartinib and drug replenished at each subculturing until emergence of resistant clones at 5 weeks duration in culture (FIG. 9A). Genomic DNA or total RNA was isolated from quizartinib-resistant and matched parental MOLM-13 cells at the time of FACS sorting to generate bulk sequencing control libraries for comparison to single cell datasets.

FACS. For single cell analysis, ˜2.0 E6 MOLM-13 quizartinib-resistant or matched parental cells were rinsed twice in Dulbecco's Phosphate Buffered Saline (Gibco) lacking calcium and magnesium supplemented with 2% FBS and kept on ice until BD FACSAria III FACS sorting. Following Calcein AM, propidium iodide and DAPI staining, live cell gating was established (DAPI/PI negative, top 70% Calcein-AM positive) and single cells were sorted (130 micron nozzle assembly) into low-bind 96 well PCR plates (semi-skirted) containing cell buffer and immediately frozen on dry ice following brief vortexing and centrifugation.

Combined genomic/transcriptomic analysis. Firstly, biotin-conjugated oligo dT primer was utilized in a template-switching reverse transcription reaction to generate first-strand cDNA from single MOLM-13 parental or quizartinib-resistant cells. Primary Template-directed Amplification (PTA) was performed in succession following reverse transcription. First-strand cDNA was then affinity-purified using streptavidin M-280 beads and subjected to two high-salt washes followed by one low-salt wash. 20-cycles of preamplification was performed to generate 2nd strand cDNA and RNA sequencing libraries were prepared using a Nextera DNA Flex Library Preparation Kit. For preparation of PTA libraries, PTA product not bound to streptavidin beads was purified using beads and ligated to TruSeq adapters. Amplification products from PTA reactions were first purified by bead cleanup, measured by Qubit, and analyzed by electrophoresis. Typical yields for mammalian cells (˜6 pg DNA) were 1-3 ug, where single bacterial genomes (2-4 fg) generated up to 50 ng. The amplicon product size for samples amplified by PTA were between 0.2-4 kB (average of 1.5Kb). PTA libraries were prepared without fragmentation for WGS methods and resulted in yields of approximately 500 ng, with a size range of 300-550 bases. Whole genome from mammalian cells were analyzed by NovaSeq targeting ˜550 million reads. Sequencing files are then transferred for trimming alignment and VCF file creation and analyzed by the Trailblazer™ cloud based bioinformatic platform solution. QC and library prep time was 4-6 hrs. Parallel experiments were conducted using RNASeq alone for comparison.

Results. RNA expression from both parental and resistant cultures demonstrated the ability to create cDNA pools (FIG. 9B) using the single-pot RNA seq chemistry and the genes expressed in these cells created distinct patterns that enable visualization of the cell populations by gene expression over the average of ˜10K genes detected per cell. In a separate workflow, single-cell genomes were amplified using the PTA method. The two protocols were then combined (yields in FIG. 9D) to produce combined transcriptome and genome cDNA pools from each cell. Low pass (˜5 million reads/cell) demonstrate effective amplification and library preparation of both resistant and parental lines, having low mitochondrial chromosome amounts and high complete PreSeq genome estimates (FIGS. 10A-10C). The data demonstrated the transcripts generated during the RT step are not effectively amplified by the PTA reaction compared to DNA and that the DNA in the single cells is effectively amplified using the combined protocol, compared to standard PTA amplified genomes from single cells (FIG. 9D). The combined RNASeq/PTA method generated similar results (FIG. 10A) to the standard PTA protocol where the ChrM and percent duplicates was typically less than 2% and the estimate genome size was greater than 3 billion bases (FIGS. 10A-10C). Evaluation of genomes revealed mapping and coverage over 90%, and specific calling of over 75% of single nucleotide variants in each cell. More variation was observed in the dual protocol compared to the standard PTA Genome chemistry. For the transcriptome the prototype chemistry appeared to detect ˜3000-5000 genes that contained an exon-exon junction. ˜30% in the genes were detected in the dual protocol (FIG. 10D) compared to the RNAseq-only protocol (FIG. 9C). Additionally, the dual/combined RNASeq/PTA protocol was used with a second resistant cell line SUM159 (Triple negative breast cancer cell line). RNAseq data run in both protocols generated similar PCA distribution, indicating the combined chemistry is able to detect differential gene expression that is not limited to a single-cell type of parental and resistant cells. (FIGS. 10E-10F).

Deep sequencing of 7 parental and 5 resistant molm13 cells was performed to an approximate depth of 25× (FIG. 11). The reads were aligned to Hg38 using bwa mem. Quality control and SNV-calling was performed using GATK4 best practices. SNVs were only considered if they were restricted to at least 2 resistant cells, no alternative alleles were called in any parental cell, and at least 6 parental cells were genotyped. All cells had at least 96% of the genome covered at 1× coverage and at least 76% covered at 10×. The inset shows that the known Flt3 indel in molm13 cells is detected in all cells (4 shown for clarity).

The RNAseq and PTA methods are generally comparable, where both mapping and coverage exceeded 95%, and ChrM and PCR duplicates were generally below 2.0%. Additionally, over 95% of the genome in select samples of both sum 159 parental and resistant cell lines was recovered. For the Molm13 cell line an overexpressed gene GAS6 (L) was identified, which is a known mechanism of quizartinib resistance. Gas6 is the ligand for AXL, which is a clinically-relevant resistance mechanism in relapsed patients who fail quizartinib treatment (FIG. 11B). Deep genome sequencing of both the parental and resistant MOLM13 cell lines from the dual protocol, detected mutations that were distributed across all chromosomes. Collectively, among all single cells, 5675 SNVs unique to the quizartinib-resistant population were identified. Coding sequence variation was detected, however the majority of the observed variants were in intergenic space. Without being bound by theory, while passenger mutations are undoubtedly present in this variant cohort, this suggests that regulation of gene expression at the enhancer or promoter level is contributing to resistance as well as potentially the regulation of non-coding RNAs. The dual mRNA seq transcriptome chemistry/PTA has the ability to detect over 10K genes in a single cell, which can be enriched by FACS. The PTA method has the ability to recover over 97% of the complete genome of an individual cell. The ability to recover both the transcriptome and the genome does not significantly affect sensitivity on the ability to recover the majority of the genome. Over 70% of the gene expressed can be detected in many of the cells when comparing the transcriptome only or combined transcriptome/genome amplification chemistry.

### Example 10

### PTA Single Cell Analysis with Exome Capture

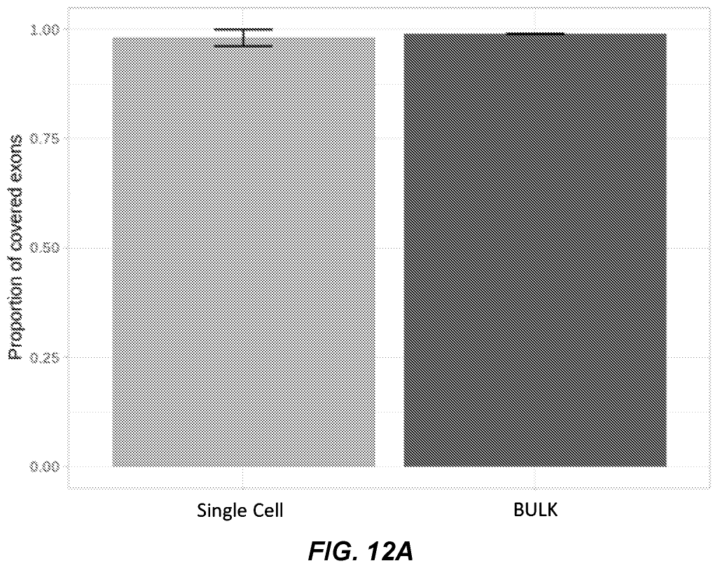

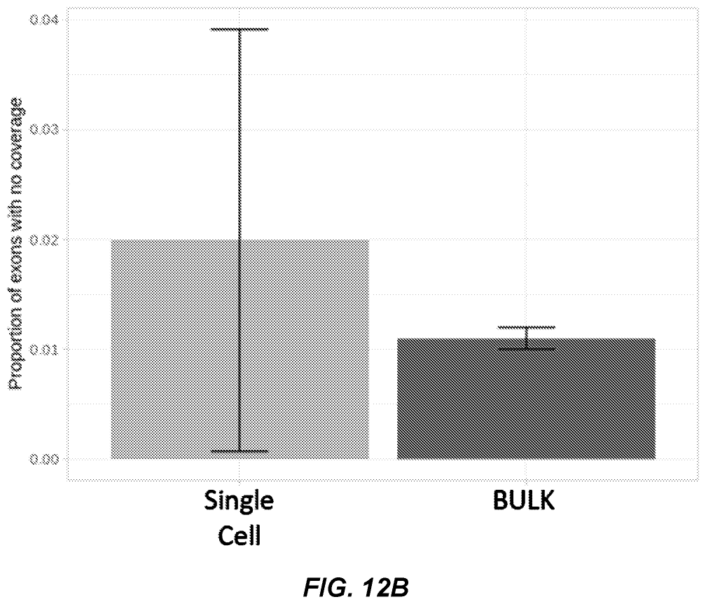

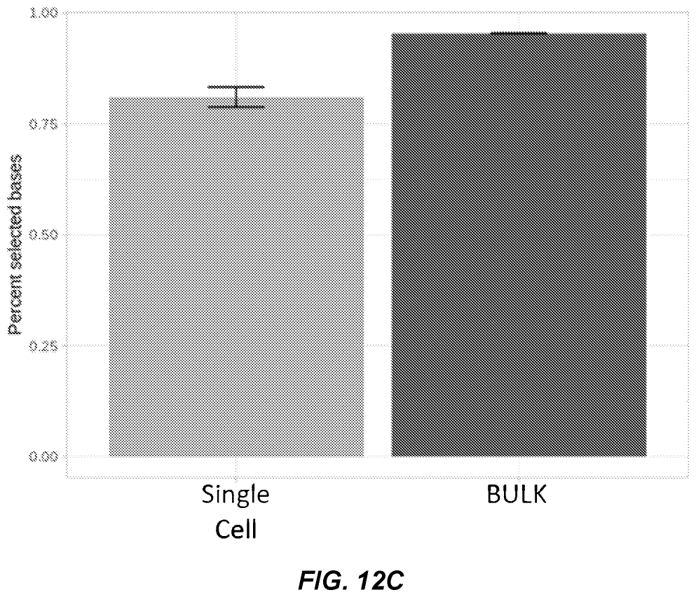

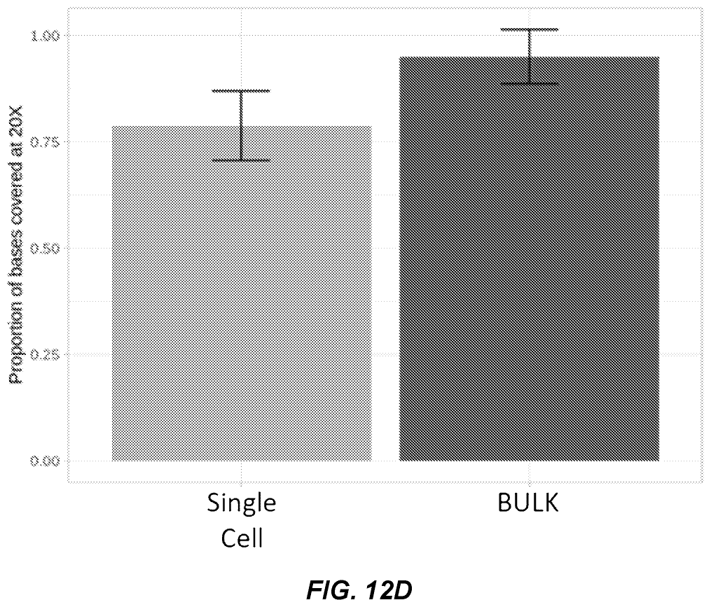

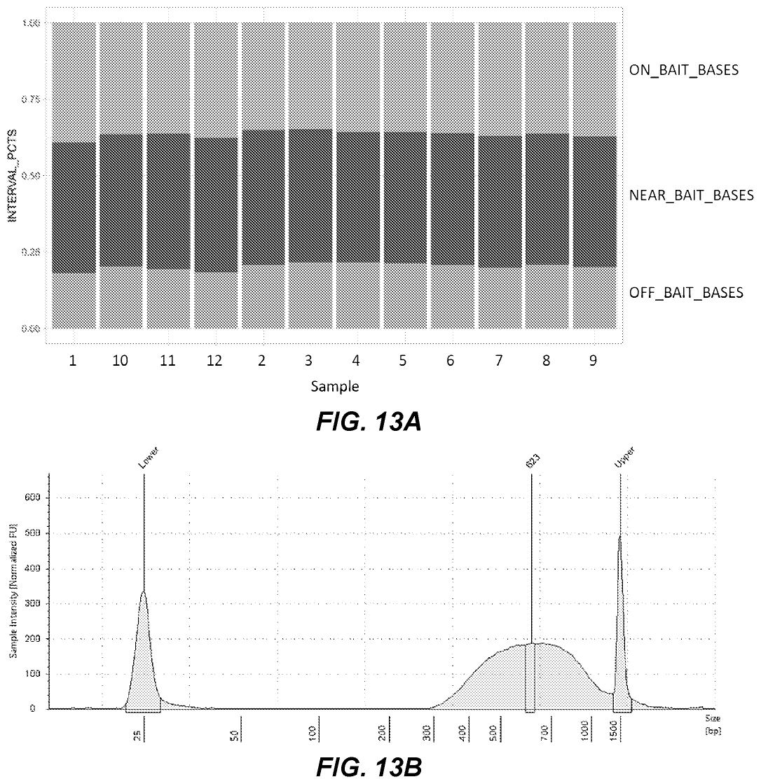

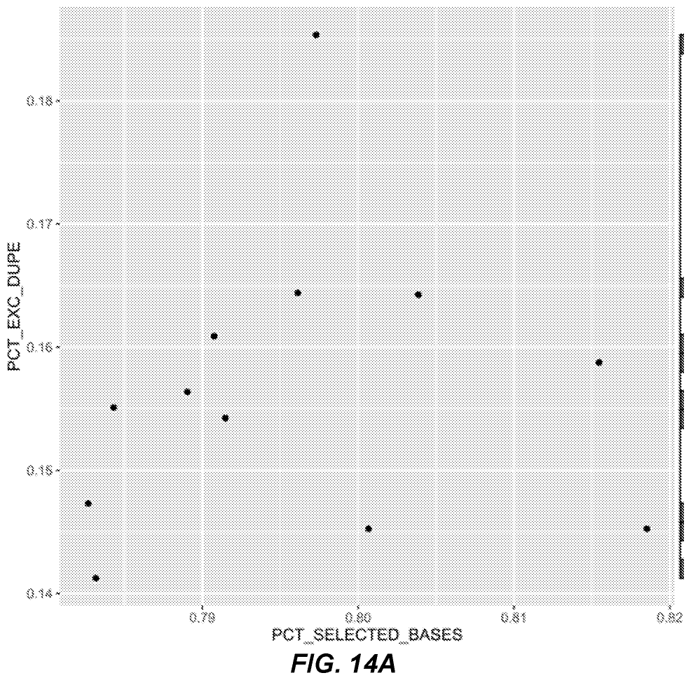

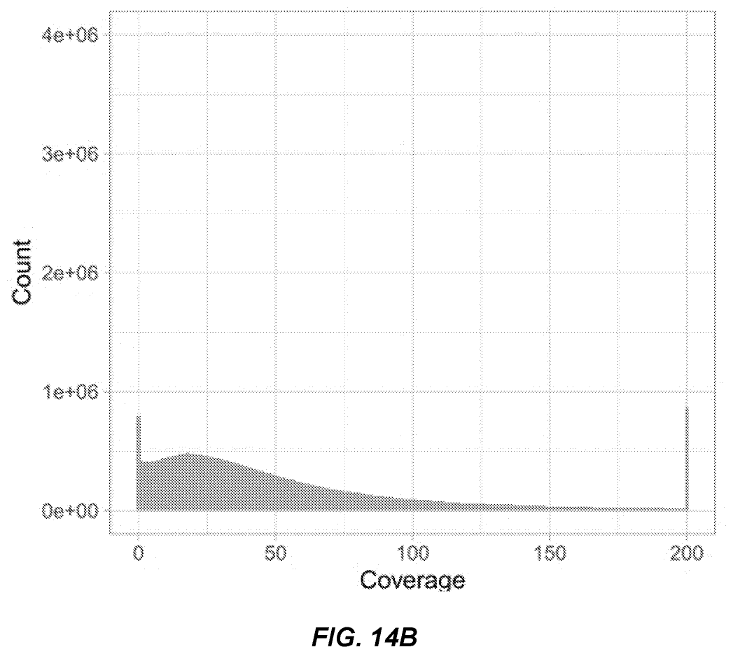

The general PTA methods of Example 3 were used with modification: an additional exome capture step was utilized to enrich PTA-generated amplicons. 60 million reads were obtained for both single cell samples (27 samples) and bulk sample (112 samples). Exome capture sequencing results from single cells were compared to those of the bulk sample (FIGS. 12A-12D, 13A, 14A, and 14B). Sequencing results were consistent across multiple samples (FIG. 13A), and the average size of captured amplicons was 623 bases (FIG. 13B).

### Example 11

### Exome Capture+Multiomics

The general method of any of Examples 5-8 is used with modification: an additional capture step is utilized to enrich PTA-generated amplicons generated from genomic DNA. The capture step includes either an exome panel or other panel targeting specific genes. In some instances, such panels are directed to cancer hot spots, viral genomes, or mitochondrial DNA.

The examples described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of multiomic single-cell analysis comprising:
a. isolating a single cell from a population of cells;
b. sequencing a cDNA library comprising polynucleotides amplified from mRNA transcripts from the single cell; and
c. sequencing a genome of the single cell, wherein sequencing the genome comprises:
i. contacting the genome with at least one amplification primer, at least one nucleic acid polymerase, and a mixture of nucleotides, wherein the mixture of nucleotides comprises at least one terminator nucleotide which terminates nucleic acid replication by the polymerase, and
ii. amplifying at least some of the genome to generate a plurality of terminated amplification products, wherein the replication proceeds by strand displacement replication;
iii. ligating the molecules obtained in step (ii) to adaptors, thereby generating a genomic DNA library; and
iv. sequencing the genomic DNA library.

2. (canceled)

3. (canceled)

4. The method of claim 1, wherein sequencing a cDNA library comprises amplification of mRNA transcripts with template-switching primers.

5. The method of claim 1, wherein at least some of the polynucleotides of the cDNA library comprise a barcode.

6. (canceled)

7. (canceled)

8. (canceled)

9. (canceled)

10. (canceled)

11. The method of claim 1, wherein the single cell is isolated by flow cytometry.

12. The method of claim 1, wherein the method further comprises removing at least one terminator nucleotide from the terminated amplification products.

13. (canceled)

14. The method of claim 1, wherein the plurality of terminated amplification products are 250-1500 bases in length.

15. The method of claim 1, wherein the plurality of terminated amplification products comprise at least 97% of the single cell's genome.

16. The method of claim 1, wherein at least some of the amplification products comprise a cell barcode or a sample barcode.

17. The method of claim 1, wherein sequencing a cDNA library comprises cystolic lysis of the single cell, and reverse transcription.

18. (canceled)

19. The method of claim 1, wherein the cDNA library comprises at least 500 genes.

20. The method of claim 1, wherein sequencing a genome of the single cell further comprises nuclear lysis of the single cell.

21. (canceled)

22. The method of claim 1, wherein at least one mutation is identified in the genome of the cell, wherein the mutation differs from a corresponding position in a reference sequence.

23. The method of claim 1, wherein the at least one mutation occurs in less than 1% of the population of cells.

24. (canceled)

25. (canceled)

26. The method of claim 1, wherein the at least one mutation occurs in no more than 1% of the amplification product sequences.

27. (canceled)

28. (canceled)

29. A method of multiomic single-cell analysis comprising:
a. isolating a single cell from a population of cells;
b. identifying at least one protein on the surface of the single cell; and
c. sequencing a genome of the single cell, wherein sequencing the genome comprises:
i. contacting the genome with at least one amplification primer, at least one nucleic acid polymerase, and a mixture of nucleotides, wherein the mixture of nucleotides comprises at least one terminator nucleotide which terminates nucleic acid replication by the polymerase;
ii. amplifying at least some of the genome to generate a plurality of terminated amplification products, wherein the replication proceeds by strand displacement replication;
iii. ligating the molecules obtained in step (ii) to adaptors, thereby generating a genomic DNA library; and
iv. sequencing the genomic DNA library.

30. The method of claim 29, wherein identifying at least one protein on the cell surface comprises contacting the cell with a labeled antibody which binds to the at least one protein.

31. The method of claim 30, wherein the labeled antibody comprises at least one fluorescent label or mass-tag.

32. The method of claim 30, wherein the labeled antibody comprises at least one nucleic acid barcode.

33. A method of multiomic single-cell analysis comprising:
a. isolating a single cell from a population of cells;
b. sequencing a genome of the single cell, wherein sequencing the genome of the cell comprises:
i. digesting the genome with a methylation-sensitive restriction enzyme to generate genomic fragments;
ii. contacting at least some of the genomic fragments with at least one amplification primer, at least one nucleic acid polymerase, and a mixture of nucleotides, wherein the mixture of nucleotides comprises at least one terminator nucleotide which terminates nucleic acid replication by the polymerase;
iii. amplifying at least some of the genome to generate a plurality of terminated amplification products, wherein the replication proceeds by strand displacement replication;
iv. amplifying at least some of the genomic fragments with methylation-specific PCR;
v. ligating the molecules obtained in steps (iii and iv) to adaptors, thereby generating a genomic DNA library and a methylome DNA library; and
vi. sequencing the genomic DNA library and the methylome library.
